SEQUENCE LISTING (32 sequences)

SEQ ID NO: 1 - DNA, 1551 bp, Homo sapiens
atggctggcagtcttacaggtctcctgctcctgcaagctgtctcttgggcttctggggccaggccctgtatccccaaatcctttggatactcatctgtggtgtgtgtttgtaatgccacttattgtgatagctttgacccccccacctttcctgcactgggcaccttttcaaggtatgaatctaccaggtctgggaggaggatggagctgagtatggggcccatccaagcaaaccatactggcactggcttgctgctgacactgcaacctgaacagaagttccagaaagtgaagggctttggaggagccatgactgatgctgctgccctcaatattttggccctgagcccccctgctcagaatctccttttgaaatcatacttctctgaggagggaattggatacaatatcatcagggtgccaatggcctcatgtgactttagtattaggacttacacctatgctgatacccctgatgatttccagctgcataacttctcattgcctgaggaggataccaaattgaagatcccactcattcacagggccctgcaactggctcagagaccagtgtcattgctggcctccccctggacctccccaacttggctcaaaaccaatggggctgtcaatggtaagggctctcttaaggggcagcctggagacatttaccatcagacctgggccaggtattttgtgaagttcctggatgcttatgctgagcacaaattgcaattttgggctgttacagctgagaatgaaccctctgcaggactgctgtctggctatcctttccagtgcctgggctttacccctgagcatcagagggatttcattgccagggacctgggacctactcttgccaatagcacacaccataatgtgaggcttctgatgcttgatgaccagagacttctgctgccacactgggccaaggttgtcctgacagatcctgaggctgccaagtatgttcatgggattgctgtgcactggtatctggacttccttgctccagctaaggccaccctgggagaaacacacaggttgtttcccaatacaatgctttttgcatcagaggcctgtgtgggcagtaaattttgggagcagtctgttaggctggggagctgggatagaggaatgcaatactcccattctatcatcaccaatctgctctaccatgtggtggggtggactgactggaaccttgcccttaaccctgagggtggccccaattgggtcaggaattttgtggatagtcccatcattgtggatatcaccaaggacacattctataagcaaccaatgttctatcacctgggtcactttagtaagtttatccctgaggggtcccagagggtgggactggtggcttcccagaagaatgatctggatgctgtggccctgatgcaccctgatggcagtgctgtggttgttgttctcaatagaagctctaaagatgtgcccttgaccatcaaagatccagctgtgggatttctggaaacaatttcccctggttatagcatccacacttacctttggagaaggcagtga

SEQ ID NO: 2 - DNA, 388 bp, Homo sapiens
attcctgctgggaaaagcaagtggaggtgctccttgaagaaacagggggatcccaccgatctcaggggttctgttctggcctgcggccctggatcgtccagcctgggtcggggtggggagcagacctcgcccttatcggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatggcccgggct

SEQ ID NO: 3 - DNA, 208 bp, Bos taurus
gccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcact

SEQ ID NO: 4 - DNA, 5295 bp, Artificial Sequence
  synthetic polynucleotide (Recombinant Construct)
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagaggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacagatctgaattcctgctgggaaaagcaagtggaggtgctccttgaagaaacagggggatcccaccgatctcaggggttctgttctggcctgcggccctggatcgtccagcctgggtcggggtggggagcagacctcgcccttatcggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatggcccgggctgcagctctaaggtaaatataaaatttttaagtgtataatgtgttaaactactgattctaattgtttctctcttttagattccaacctttggaactcaattcagccaccatggctggcagtcttacaggtctcctgctcctgcaagctgtctcttgggcttctggggccaggccctgtatccccaaatcctttggatactcatctgtggtgtgtgtttgtaatgccacttattgtgatagctttgacccccccacctttcctgcactgggcaccttttcaaggtatgaatctaccaggtctgggaggaggatggagctgagtatggggcccatccaagcaaaccatactggcactggcttgctgctgacactgcaacctgaacagaagttccagaaagtgaagggctttggaggagccatgactgatgctgctgccctcaatattttggccctgagcccccctgctcagaatctccttttgaaatcatacttctctgaggagggaattggatacaatatcatcagggtgccaatggcctcatgtgactttagtattaggacttacacctatgctgatacccctgatgatttccagctgcataacttctcattgcctgaggaggataccaaattgaagatcccactcattcacagggccctgcaactggctcagagaccagtgtcattgctggcctccccctggacctccccaacttggctcaaaaccaatggggctgtcaatggtaagggctctcttaaggggcagcctggagacatttaccatcagacctgggccaggtattttgtgaagttcctggatgcttatgctgagcacaaattgcaattttgggctgttacagctgagaatgaaccctctgcaggactgctgtctggctatcctttccagtgcctgggctttacccctgagcatcagagggatttcattgccagggacctgggacctactcttgccaatagcacacaccataatgtgaggcttctgatgcttgatgaccagagacttctgctgccacactgggccaaggttgtcctgacagatcctgaggctgccaagtatgttcatgggattgctgtgcactggtatctggacttccttgctccagctaaggccaccctgggagaaacacacaggttgtttcccaatacaatgctttttgcatcagaggcctgtgtgggcagtaaattttgggagcagtctgttaggctggggagctgggatagaggaatgcaatactcccattctatcatcaccaatctgctctaccatgtggtggggtggactgactggaaccttgcccttaaccctgagggtggccccaattgggtcaggaattttgtggatagtcccatcattgtggatatcaccaaggacacattctataagcaaccaatgttctatcacctgggtcactttagtaagtttatccctgaggggtcccagagggtgggactggtggcttcccagaagaatgatctggatgctgtggccctgatgcaccctgatggcagtgctgtggttgttgttctcaatagaagctctaaagatgtgcccttgaccatcaaagatccagctgtgggatttctggaaacaatttcccctggttatagcatccacacttacctttggagaaggcagtgaaaatgaaggcctgataattgcaccaccaggcctgataggccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcactagtccactccctctctgcgcgctcgctcgctcactgaggccgggcgaccaaaggtcgcccgacgcccgggctttgcccgggcggcctcagtgagcgagcgagcgcgcagagagggatctagatatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - protein, 497 aa, Homo sapiens
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 6 - protein, 516 aa, Homo sapiens
MAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 7 - DNA, 1611 bp, Homo sapiens
atggagttttcaagtccttccagagaggaatgtcccaagcctttgagtagggtaagcatcatggctggcagcctcacaggtttgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatcgccagtga

SEQ ID NO: 8 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 9 - DNA, 644 bp, Artificial Sequence
  synthetic polynucleotide (Recombinant construct)
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 10 - DNA, 720 bp, Aequorea victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 11 - DNA, 1611 bp, Artificial sequence
  synthetic polynucleotide (Human GBA1 coding nucleotide sequence (IDT optimized sequence))
atggagttctcatctccctcacgagaagaatgtccgaaacctctttcaagagtaagcatcatggccggcagcttgaccggtcttttgttgttgcaggccgtgtcctgggcctcaggtgctaggccatgcattcctaaatccttcggctatagtagcgtggtttgcgtctgcaacgccacatactgtgacagtttcgatccacctaccttcccagcgctgggtaccttctcacggtatgaatcaacgcgatcagggcgcagaatggaactttcaatggggccaatccaagctaaccacacgggaacgggtcttctgctgacgctccaaccggaacaaaagttccaaaaggtaaaaggctttggaggtgcgatgactgatgccgcagcactcaacatcctggcgctctcaccgccggcacaaaatttgctgttgaagagttatttctcagaagaagggatcggttacaacatcatacgggtcccgatggcgagctgtgacttttctataagaacatatacctatgcggatacgcccgacgatttccaacttcataattttagtctgcctgaggaagacacaaagttgaagataccgctgatacacagagcattgcagcttgctcaacgaccggtcagcttgcttgccagcccatggacaagtccaacatggcttaagaccaatggcgcggttaatggcaagggatccctgaagggccagccgggagacatctatcatcaaacttgggcgcggtattttgtcaagttcttggacgcctacgctgagcacaaactgcagttctgggccgttaccgccgaaaatgaaccatccgccggactgctttctggctaccctttccaatgtcttggctttacgcctgaacaccaaagagacttcattgctcgggaccttggtccaacgctcgcgaacagtactcatcataatgtacgactcttgatgctcgatgaccagcgactgttgcttccacattgggccaaggtagttctgaccgaccccgaagccgctaaatacgtccacggcattgctgtccattggtaccttgactttttggctcccgcaaaagccactctgggtgaaacacacagactctttccaaacacgatgcttttcgcatcagaagcctgcgtcggaagtaaattttgggaacagtcagtaaggttgggtagttgggatcgcgggatgcaatatagtcatagcattattaccaacttgctttatcacgtcgttgggtggacagattggaacctcgcgttgaatcctgaaggcggccctaattgggtaagaaactttgttgattcacctattatcgtcgacataaccaaggacacattctacaagcaaccgatgttctatcaccttgggcatttcagtaaattcataccagagggcagccagcgcgtcgggttggtagcctctcaaaaaaacgatttggatgcggtcgctctgatgcatcccgacgggagcgcagtagtcgttgtccttaaccgaagctccaaggatgtacccctcacgattaaggaccctgctgtcgggttccttgaaactataagtcccggctatagtattcatacttatctctggagaagacagtga

SEQ ID NO: 12 - DNA, 1611 bp, Artificial sequence
  synthetic polynucleotide (Human GBA1 coding nucleotide sequence (GenScript optimized sequence))
atggagttttcaagcccctcacgggaagagtgccctaagcccctgtcacgggtctcaattatggccgggagcctgactggcctgctgctgctgcaggccgtgagctgggcatcaggagccaggccttgcatcccaaagtctttcggctacagctccgtggtgtgcgtgtgcaacgccacctattgtgactccttcgatccccctacctttcccgccctgggcacattttctagatacgagtctacacgcagcggccggagaatggagctgagcatgggccctatccaggccaatcacaccggaacaggcctgctgctgaccctgcagccagagcagaagttccagaaggtgaagggctttggaggagcaatgacagacgcagccgccctgaacatcctggccctgtccccacccgcccagaatctgctgctgaagtcctacttctctgaggagggcatcggctataacatcatccgggtgcccatggccagctgcgacttttccatcagaacctacacatatgccgatacccctgacgatttccagctgcacaatttttccctgccagaggaggatacaaagctgaagatccccctgatccaccgggccctgcagctggcacagcggcccgtgagcctgctggccagcccctggacctcccctacatggctgaagaccaacggcgccgtgaatggcaagggctctctgaagggacagccaggcgacatctaccaccagacatgggccagatatttcgtgaagtttctggatgcctacgccgagcacaagctgcagttctgggccgtgaccgcagagaacgagccttctgccggcctgctgagcggctatcccttccagtgcctgggctttacacctgagcaccagcgggactttatcgccagagatctgggcccaaccctggccaactccacacaccacaatgtgaggctgctgatgctggacgatcagcgcctgctgctgcctcactgggccaaggtggtgctgaccgacccagaggccgccaagtacgtgcacggcatcgccgtgcactggtatctggatttcctggcaccagcaaaggccaccctgggagagacacaccggctgttccctaacaccatgctgtttgccagcgaggcctgcgtgggctccaagttttgggagcagtccgtgaggctgggatcttgggacaggggcatgcagtactcccactctatcatcaccaatctgctgtatcacgtggtgggctggacagactggaacctggccctgaatccagagggcggccccaactgggtgagaaatttcgtggatagccccatcatcgtggacatcaccaaggatacattctacaagcagccaatgttttatcacctgggccacttctctaagtttatcccagagggcagccagagggtgggcctggtggccagccagaagaacgacctggatgccgtggccctgatgcaccctgatggctccgccgtggtggtggtgctgaatcgctctagcaaggacgtgcctctgaccatcaaggatccagccgtgggcttcctggagactatttcccccggctattcaattcatacctatctgtggagaaggcagtga

SEQ ID NO: 13 - DNA, 448 bp, homo sapiens
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 14 - protein, 735 aa, Artificial sequence
  synthetic polypeptide (AAV TT capsid protein )
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKSGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 15 - DNA, 145 bp, Artificial sequence
  synthetic polynucleotide (Flip ITR of AAV2)
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 16 - DNA, 145 bp, Artificial sequence
  synthetic polynucleotide (Flop ITR of AAV2 )
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 17 - protein, 449 aa, homo sapiens
MELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 18 - protein, 449 aa, homo sapiens; identical to SEQ ID NO: 17

SEQ ID NO: 19 - DNA, 1611 bp, Artificial sequence
  synthetic polynucleotide (human GBA1 gene variant)
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 20 - DNA, 376 bp, Artificial sequence
  synthetic polynucleotide (nGUSB promoter #2)
attcctgctgggaaaagcaagtggaggtgctccttgaagaaacagggggatcccaccgatctcaggggttctgttctggcctgcggccctggatcgtccagcctgggtcggggtggggagcagacctcgcccttatcggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagc

SEQ ID NO: 21 - DNA, 1663 bp, artificial sequence
  synthetic polynucleotide (CAG promoter #2)
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 22 - DNA, 400 bp, Artificial sequence
  synthetic polynucleotide (Human Ubiquitin C (UbC) promoter short version)
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggt

SEQ ID NO: 23 - DNA, 1212 bp, Artificial sequence
  Synthetic polynucleotide (Human Ubiquitin C (UbC) promoter long version)
ccggaggcgcggcccaaaaccgcggagggcgcccgcggggggaggagtgccgctcgcgacggtgcagtctgcttcccgcgtcgctcgcaggactaggaaggcgggcctgcgagtcctgtcgccgggcgacgagtattctgagccggaatcttggggtcatagtcgtcttcctgtaaaatcctgccctgaacccactgagatcccgtgaccaaaagaaaggtctctcgccttgtccgctccttttcatcagggaagagccgctaagacgcctccctagaggcaccccgccacttgcggctactaatatattcctgcgcggcccacaccgtgtcgatcaaggcagcgtcggccctaaacccagcgccaagaacaaacacctagcgacactagcagtgaaccactcatcgcccgacgacccgaccggccccgaaagcaccggcggcccggcgagccaccctgccttcgcacacctctctggcggttcccgacatcagacccaggcgctcgttccaacgggacttgacccccaacccccctcgcgtcgttttaccgccgacaagggctcagaacttaccttctgcgaacactccgcccgacactccagcaactttgttccaccccccgtaccacccgccgttcttgggttccagaactccggaagcgattacgccctttcgagaataagcccactctacccgaccccgtggtagacccctgggactgcacttcaaacagtgactgacctcttgagccaaacagcagacaacgcccccgccgtcaataccgccacggcaacccgtcacgtgggcatggaaaccctcgcgcgcgggagcagcacagcactgcagtgggcaagacaaccgaatattacgtcccaccccggtggacggccatccacacgccatccgaaaagaggcagcgtcctgcgtcccaagcccggatcccatccgagaggacttagctgtccgcggcctggagaccactcccctccctattcactccgcagtcaaagaaaccagccaaaatacatggatagaagaattcatcgacttcgaggccaaaacttgatacgcgagccccaaccgctcacacaaaacacttcaaaaaatccgtggaaaactttacattagtaaacccagttatacattaaaagtcacaatctgatcatttaacaggcgatttaagaccggcaaaaaccgaaaaaacaatctg

SEQ ID NO: 24 - DNA, 511 bp, Artificial sequence
  synthetic polynucleotide (Phosphoglycerate kinase 1 (PGK) promoter)
gacccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaacagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccgcggcgacgcaaagggccttggtgcgggtctcgtcggcgcagggacgcgtttgggtcccgacggaaccttttccgcgttggggttgggg

SEQ ID NO: 25 - DNA, 741 bp, Artificial sequence
  synthetic polynucleotide (CBA/CBh promoter)
agatgtactgccaagtaggaaagtcccgtaaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaaccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggaactgaaaaaccagaaagttaactggtaagtttagtctttttgtcttttatttcaggtcctggtggtgcaaatcaaagaactgctcctcagtggatgttgcctttacttctaggcctgtacggaagtgttacttctgctctaaaagct

SEQ ID NO: 26 - DNA, 818 bp, Artificial sequence
  synthetic polynucleotide (CBA/CBh promoter)
agatgtactgccaagtaggaaagtcccgtaaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaaccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggggagtcgctgcgttgccttcgccccgtgccccgctccgcgccgcctcgcgccgcccgccccggctctgactgaccgcgttactcccacaggtgagcgggcgggacggcccttctcctccgggctgtaattagcaagaggtaagggtttaagggatggttggttggtggggtattaatgtttaattacctgttttacaggcctgaaatcacttggttttaggttgg

SEQ ID NO: 27 - DNA, 720 bp, artificial sequence
  synthetic polynucleotide (GFP encoding nucleotide sequence #2)
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgaggacgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 28 - DNA, 1212 bp, artificial sequence
  synthetic polynucleotide (Human Ubiquitin C (UbC) promoter long version #2)
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 29 - DNA, 511 bp, artificial sequence
  synthetic polynucleotide (Phosphoglycerate kinase 1 (PGK) promoter #2)
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 30 - DNA, 794 bp, artificial sequence
  synthetic polynucleotide (CBA/CBh promoter #3)
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 31 - protein, 735 aa, artificial sequence [sequence omitted]

SEQ ID NO: 32 - protein, 736 aa, artificial sequence
  synthetic polypeptide (AAV9 capsid protein)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

(19) United States
(12) Patent Application Publication
(10) Pub. No.: US 2023/0277687 A1
(43) Pub. Date: Sep. 7, 2023

(54) VIRAL PARTICLES FOR USE IN TREATING TAUOPATHIES SUCH AS ALZHEIMER'S DISEASES BY GENE THERAPY

(71) Applicants: Fundacion Para La Investigacion Medica Aplicada, Pamplona (ES); CONSORCIO CENTRO DE INVESTIGACIÓN BIOMÉDICA EN RED DEL ÁREA DE ENFERMEDADES NEURODEGENERATIVAS M.P C, MADRID (ES); UCB BIOPHARMA SRL, Brussels (BE)

(72) Inventors: Gloria GONZÁLEZ-ASEGUINOLAZA, Pamplona (ES); Diego SUCUNZA GUIBERT, Pamplona (ES); José Luis LANCIEGO PÉREZ, Pamplona (ES); Ralph Michael LINDEN, Slough, Berkshire (GB)

(21) Appl. No.: 18/006,056

(22) PCT Filed: Aug. 6, 2021
(86) PCT No.: PCT/EP2021/072074

§ 371 Date: Jan. 19, 2023

(30) Foreign Application Priority Data
Aug. 6, 2020 (WO) PCT/EP2020/072091

(51) Int. Cl. A61K 48/00; C12N 9/24; C12N 15/86; A61K 38/47; A61K 49/00; A61P 25/28

(52) U.S. Cl. CPC A61K 48/0058; C12N 9/2402; C12Y 302/01045; C12N 15/86; A61K 38/47; A61K 48/0041; A61K 49/0004; A61P 25/28; C12N 2750/14143; C12N 2750/14171

(57) ABSTRACT

The present disclosure relates to viral particles for use in treating tauopathies, particularly Alzheimer's disease, by gene therapy. More specifically, the present invention relates to a viral particle for use in treating tauopathies by gene therapy in a subject in need thereof, said viral particle comprising a nucleic acid construct including a transgene encoding a glucocerebro sidase.

### TECHNICAL FIELD

The present disclosure relates to viral particles for use in treating tauopathies, particularly Alzheimer's disease, by gene therapy. More specifically, the present invention relates to a viral particle for use in treating tauopathies by gene therapy in a subject in need thereof, said viral particle comprising a nucleic acid construct including a transgene encoding a glucocerebrosidase.

### BACKGROUND ART

Neurodegenerative diseases are a heterogeneous group of unrelenting brain disorders characterized by the pathological aggregation of misfolded proteins, currently viewed as neurodegenerative proteinopathies overall. Neurodegenerative diseases can be broadly categorized into two main groups, namely synucleinopathies and tauopathies, as a function of the type of proteinaceous aggregates that are typically found in these disorders (see Table 1 below).

TABLE 1
Examples of typical proteinopathies.

| Type of | | Neurodegenerative |
|---|---|---|
| proteinopathy | Aggregated protein | disease |
| Tauopathy | Hyperphosphorylated | Fronto-temporal lobar |
| | Tau | degeneration (FTD) |
| | | Alzheimer's disease (AD) |
| | | Progressive supranuclear |
| | | palsy (PSP) |
| | | Corticobasal degeneration |
| | | (CBD) |
| | | Tangle predominant dementia |
| | | (TPD) |
| | | Guam Parkinson dementia |
| | | complex |
| | | Argyrophilic grain disease |
| | | (AGD) |
| | | Pick's disease (AD unrelated) |
| Synucleinopathy | α-synuclein | Parkinson's disease (PD) |
| | | Dementia with Lewy bodies |
| | | (DLB) |
| | | Multiple system atrophy |
| | | (MSA) |
| Huntingtin | Huntingtin | Huntington disease (HD) |

Modified from: Bayer T. A.. European Neuropsychopharmacology (2015) 25, 713-724

Besides alpha-synuclein and tau, protein aggregates representing the main neuropathological hallmark of neurodegenerative diseases have also been described for Huntington's disease (huntingtin protein aggregates) and amyotrophic lateral sclerosis (ALS; ubiquitin aggregates). It is also worth noting that most of these disorders share two common features: first, the initial degenerative insult is restricted to specific areas of the brain, such as for instance the substantia nigra pars compacta in Parkinson's disease (PD) and the nucleus basalis of Meynert, locus ceruleus and entorhinal cortex when considering Alzheimer's disease (AD). As disease progresses, protein aggregates (alpha-synuclein in PD, tau in AD) spread in a “prion-like” fashion to more extensive brain locations by taking advantage of cortical circuits, ultimately leading to a broader proteinopathy throughout the brain, setting the ground for clinical progression of the symptoms and signs that typically characterize neurodegenerative diseases. Although at present the ultimate mechanisms through which tau aggregation promotes neuronal death remain to be fully characterized, neuronal death is largely viewed as a two-step phenomenon, initially triggered by the intracellular aggregation of tau in the form of neurofibrillary tangles, later followed by activation of microglial cells. Activated microglial cells release pro-inflammatory cytokines, further enhancing and perpetuating neuronal death. In other words, when coming to design any potential disease-modifying treatment, the approach to be implemented would require the simultaneous targeting of two concurrent processes, i.e. an efficient clearance of misfolded tau together with an attenuation of microglial-driven pro-inflammatory phenomena.

Besides a minimal percentage of familial cases, AD and related tauopathies are largely viewed as sporadic disorders, meaning that they occur randomly and cannot be attributed to genetic causes. For sporadic AD, a genetic susceptibility has been described, particularly related to the inheritance of the APOE4 allele that has been described in significant percentages of sporadic AD cases. It has been estimated that around 0.1% of AD cases are familial forms of autosomal dominant inheritance that can be attributed to mutations in genes coding for the amyloid precursor protein (APP) and presenilins 1 and 2. Neurons die upon the progressive intracellular aggregation of misfolded tau protein, these aggregates being known neurofibrillary tangles. The distribution of neurofibrillary tangles in AD is defined by Braak stages (Braak and Braak, Acta Neuropathol 1991; 82:239-259). For stages I and II, neurofibrillary tangles are restricted to discrete brain regions such as the basal forebrain and entorhinal cortex. Limbic regions became engaged in stages III and IV, whereas for stages V and VI there is a more extensive neocortical pathology. Although the cause of AD remains largely unknown (e.g. AD is considered as an idiopathic disorder), two main hypotheses triggering the disease cascade of events have been taken into consideration, namely, the amyloid and tau hypothesis. The amyloid hypothesis postulates that the presence of extracellular amyloid plaques (made of aggregates of amyloid beta protein) is the main pathological hallmark of AD. This hypothesis is supported by the fact that APOE4 allele is the best known genetic risk factor and indeed APOE4 allele is not efficient in breaking-down amyloid beta proteins. By contrast, the tau hypothesis suggests that the pathological aggregation of hyperphosphorylated tau protein in the form of neurofibrillary tangles is the main culprit in the disease, leading to impairment of neuronal transport mechanisms and finally triggering neuronal death. Support on the tau hypothesis is provided by the fact that one of the brain regions where neurofibrillary tangles firstly became evident is the nucleus basalis of Meynert, a brain area made of cholinergic neurons. It is well known that there is a reduction on cholinergic transmission in AD and indeed the use of acetylcholinesterase inhibitors has some beneficial medical effect in early stages of AD.

At present, currently available pharmacological treatments for AD exhibit small symptomatic relief and are largely viewed as merely palliative in nature. Accordingly, the main unmet medical need is to develop disease-modifying strategies for AD and related tauopathies, intended to slow-down—or even ideally arrest—the unrelenting progressive course of these devastating brain disorders. An ideal candidate should be a method capable of conducting an efficient clearance of tau aggregates exhibiting a neuroprotective effect and ultimately impeding trans-neuronal passage of tau (prion-like spread; Maxan and Cicchetti, J Exp Neurosci 2018; 12:1-4).

Considering neurogenerative disorders other than tauopathies, such as those characterized by the intracellular aggregation of misfolded alpha-synuclein such as Parkinson's disease (PD) and dementia with Lewy bodies (DLB), it has been recently characterized that both homo- and heterozygous mutations in the GBA1 gene encoding for a lysosomal enzyme known as glucocerebrosidase (GCase) numerically represent the main genetic risk factor for PD and DLB. GBA1 mutations led to a loss-of-activity of glucocerebrosidase within the lysosome, ultimately triggering-through some unknown mechanisms the pathological aggregation of alpha-synuclein. Regarding sporadic AD, a potential engagement of GBA1 mutations in the mechanisms leading to the pathological aggregation of misfolded tau protein has not been elucidated.

As a lysosomal enzyme, glucocerebrosidase is ubiquitously expressed throughout the brain, although it has recently been identified a number of brain areas in macaques where neurons exhibit an enriched content of glucocerebrosidase (Dopeso-Reyes et al., 2018).

US2015/0284472 reports methods for preventing loss of neural function in a mammal comprising administering a therapeutically effective amount of an agent that increases glucocerebrosidase activity.

When considering the implementation of disease-modifying therapies for AD and related tauopathies using AAVs, there still remain the need to provide therapies which fulfill a number of main goals in order to properly reach the required endpoints, as follows:
Proof-of-principle that the AAV-based gene therapy induces tau clearance in neurons,
demonstration that the reduction on tau burden exerts a neuroprotective effect.

### SUMMARY

The present invention provides retrogradely-transported viral particles for use in gene therapy for treating Alzheimer's disease and related tauopathies in patients at advanced stages of the disease, where a widespread tauopathy is present throughout the brain, in particular when engaging the cerebral cortex. The present therapeutic strategies meet the above requirements, especially in view of the results obtained in mice model of sporadic Alzheimer's Disease, as well as take advantage of the use of AAV capsids modified to further enhancing the retrograde spread of a given encoded transgene.

In a first aspect, the disclosure relates to a viral particle and its use in treating tauopathy by gene therapy in a subject in need thereof, said viral particle comprising a nucleic acid construct including a transgene encoding a glucocerebrosidase.

In one embodiment, said transgene comprises a coding sequence of human glucocerebrosidase selected from the group consisting of SEQ ID NO: 5, 6, 8, 17 and 18, typically the sequence selected from the group consisting of SEQ ID NO: 1, 7, 11, 12 and 19.

In specific embodiments, said nucleic acid construct further comprises a promoter operably-linked to the transgene encoding glucocerebrosidase and wherein said promoter allows the expression of said transgene at least in neuronal and microglial cells of the substantia nigra pars compacta (SNc); and preferably also in neuronal cells of other brain areas, including at least the substantia nigra pars compacta, cerebral cortex, amygdala, and caudal intralaminar nuclei of the thalamus. Typically, said nucleic acid construct may comprise a transgene encoding a glucocerebrosidase under the control of an ubiquitous promoter, for example the GusB promoter, notably a promoter of SEQ ID NO:2 or 20, the CAG promoter of SEQ ID NO:9 or 21 or human synapsin 1 promoter (hSyn) of SEQ ID NO: 13.

In specific embodiments, said viral particle is selected among viral serotypes that simultaneously targets at least neurons and glial cells, preferably located in the cerebral cortex and subcellular structures such as the nucleus basalis of Meynert, the substantia nigra pars compacta, the locus ceruleus, the hippocampal formation and the entorhinal cortex. In more specific embodiments, said viral particle simultaneously targets at least neurons and microglial cells. More specifically, said viral particle may be selected among viral particles that simultaneously target at least dopaminergic neurons and microglial cells in the substantia nigra pars compacta.

In certain embodiments, said viral particle is selected among rAAV particles, preferably including capsid proteins selected from the group consisting of: AAV2, AAV5, AAV9, AAV-MNM004, AAV-MNM008, and AAV TT serotypes.

In a more specific embodiment, said viral particle includes AAV TT capsid protein, preferably which comprises a sequence of SEQ ID NO: 14 or sequence having at least 98.5%, preferably 99 or 99.5% identity with SEQ ID NO: 14.

In certain embodiments, said viral particle comprises viral capsid protein selected among viral variant serotypes with retrograde transport (AAVretro).

Typically, said AAVretro may be able to retrogradely disseminate in the cerebral cortex, preferably at least to the substantia nigra pars compacta and cerebral cortex after parenchymal injection in the caudate or putamen nuclei of a non-human primate as determined in an in vivo dissemination assay. Advantageously, AAVretro injected in the caudate-putamen nuclei of a non-human primate may be able to retrogradely disseminate also to other brain areas innervating the caudate-putamen nuclei, including at least substantia nigra pars compacta, cerebral cortex, amygdala, and caudal intralaminar nuclei of the thalamus.

In another aspect, the present disclosure relates to an in vivo dissemination assay includes the following steps:
a) injecting a test rAAV comprising GFP (green-fluorescent protein) encoding transgene (rAAV-GFP) by intraparenchymal injection of said rAAV-GFP into the post-commissural putamen of a non-human primate, and,
b) counting the number of GFP-expressing neurons in the cerebral cortex, preferably in brain areas innervating the caudate putamen nuclei, one month post injection, more particularly at least in the substantia nigra pars compacta, the cerebral cortex, the amygdala, and the caudal intralaminar nuclei of the thalamus.

In other embodiments, said in vivo dissemination assay further comprises a step c) of comparing the percentage of labeled neurons in the cerebral cortex, preferably in the brain areas innervating the caudate putamen nuclei with a control experiment performed with AAV-TT-GFP.

In certain embodiments, the viral particle according to the present disclosure, is advantageously selected among AAVretro particles which are able to disseminate in the cerebral cortex, preferably to at least to the substantia nigra pars compacta and the cerebral cortex, to at least the same level as AAV-TT as determined in an in vivo dissemination assay as described above.

In specific embodiments, said AAVretro capsid protein is selected among the following variant serotypes: AAV-MNM004, AAV-MNM008 and AAV-TT.

In a more specific embodiment, said AAV retro particle includes AAV TT serotype capsid protein, preferably which comprises a sequence of SEQ ID NO: 14 or sequence having at least 98.5%, preferably 99 or 99.5% identity with SEQ ID NO: 14.

In specific embodiments, said nucleic acid construct of the viral particle further comprises a polyadenylation signal sequence, notably a polyadenylation signal sequence of sequence SEQ ID NO: 3.

In specific embodiments, said nucleic acid construct is comprised in a viral vector which further comprises a 5′ITR and a 3′ITR sequences, preferably a 5′ITR and a 3′ITR sequences of an adeno-associated virus, more preferably a 5′ITR and a 3′ITR sequences from the AAV2 serotype which comprise or consist of sequence SEQ ID NO: 15 and/or 16 or a sequence having at least 80% or at least 90% of identity with SEQ ID NO: 15 and/or 16.

In specific embodiments, said nucleic acid construct comprises a nucleic acid sequence of SEQ ID NO: 4 or a nucleic acid sequence having at least 80% or at least 90% of identity with SEQ ID NO: 4.

In particular embodiments, said nucleic acid construct comprises a coding sequence of human glucocerebrosidase under the control of a promoter, allowing expression of said human glucocerebrosidase in at least both dopaminergic neurons and microglial cells, and said viral particle is selected among viral particles that targets at least dopaminergic neurons and microglial cells of the substantia nigra pars compacta, typically AAV particles including capsid proteins selected from the group consisting of AAV2, AAV5, AAV9, AAV-MNM004, AAV-MNM008, and AAV TT serotypes.

In another aspect, the disclosure relates to the use of a viral particle as described above in therapy, preferably in treating tauopathy by gene therapy in a subject in need thereof. In a specific embodiment, said tauopathy is a human sporadic tauopathy. In more specific embodiments, said tauopathy is an Alzheimer's disease, typically sporadic Alzheimer's disease.

In other embodiments, said tauopathy is a clinical entity other than Alzheimer's disease, this comprising but not limited to progressive supranuclear palsy, corticobasal degeneration, frontotemporal dementia, and Pick's disease.

In specific embodiments, said viral vector is administered to said subject by intrathecal or intraparenchymal administration, the latter preferably to brain areas such as the cerebral cortex and subcellular structures such as the nucleus basalis of Meynert, the substantia nigra pars compacta, the locus ceruleus, the hippocampal formation and the entorhinal cortex.

Said viral vector may preferably be administered to said subject by intraparenchymal administration, more preferably to the brain area of the substantia nigra pars compacta, the caudate putamen nuclei or the dentate gyrus of the hippocampal formation.

### BRIEF DESCRIPTION OF THE DRAWINGS

FIG. 1: is the amino acid sequence alignment of AAV-TT capsid protein sequence with AAV-2.

FIG. 2: is the amino acid sequence alignment of AAV-TT capsid protein sequence with AAV-9.

FIG. 3: is a schematic representation of the conducted experimental plan in mice.

FIG. 4: Immunohistochemical detection of tau protein in a coronal section of the mice brain showing the efficacy of GCase enhancement for tau clearance. Mice were firstly injected bilaterally with rAAV2/9-Tau301L. 4 weeks later, the right striatum received an intraparenchymal injection of rAAV2/9-GBA1, whereas a control-null viral vector (rAAV2/9-null) was injected into the left striatum. Animals were sacrificed 4 weeks post-delivery of rAAV2/9-GBA1 (e.g. 8 weeks post-injection of rAAV2/9-Tau301L). Obtained preliminary data demonstrate that the viral-mediated enhancement of GCase activity induced an extensive clearance of tau aggregates, both in the right cerebral cortex and striatum (panels 3 & 4, respectively), whereby the control vector did not exhibited any effect on tau pathology, as observed both in the left cerebral cortex and striatum (panels 1 & 2 respectively).

FIG. 5: Sagittal Rx plates showing the injection sites for all AAVs during ventriculography-assisted stereotaxic surgery.

FIG. 6: Representative photomicrographs showing the injection sites for all AAVs.

FIG. 7: Cartoons illustrating the injection sites for all animals (A: M295 and 296, B: 297 and 298).

FIG. 8: Biodistribution, and estimated intensities of GFP+ neurons in animals M295 (A) and M296 (B) (injected with AAV-TT-GFP). Small-sized dots (labeled as “low”) represent between 1 to 200 GFP+ cells; medium-sized dots (labeled as “moderate”) represent between 201 to 400 GFP+ cells, and large-sized dots (labeled as “high” represent more than 401 GFP+ cells.

FIG. 9: Biodistribution, and estimated intensities of GFP+ neurons in animals M297 (A) and M298 (B) (injected with AAV-9-GFP). Small-sized dots (labeled as “low”) represent between 1 to 200 GFP+ cells; medium-sized dots (labeled as “moderate”) represent between 201 to 400 GFP+ cells, and large-sized dots (labeled as “high” represent more than 401 GFP+ cells.

FIG. 10: Quantification. Histograms showing the total number of GFP+ neurons for all animals.

FIG. 11: Quantification. Histograms showing the number of GFP+ neurons for all animals across a number of regions of interest. Abbreviations: Anterior cingulate gyms (AcGg), Superior frontal gyrus (SFG), Precentral (PrG), Postcentral gyrus (PoG), Insular gyrus (Ing), Centromedian-parafascicular complex (CM-Pf), Substantia nigra pars compacta (SNc).

FIG. 12: Quantification. Histograms showing the rostrocaudal biodistribution of GFP+ neurons for all animals across a number of regions of interest of the left hemisphere. Abbreviations: Anterior cingulate gyms (AcGg), Superior frontal gyrus (SFG), Precentral (PrG), Postcentral gyms (PoG), Insular gyrus (Ing), Centromedian-parafascicular complex (CM-Pf), Substantia nigra pars compacta (SNc).

FIG. 13: Quantification. Histograms showing the rostrocaudal biodistribution of GFP+ neurons for all animals across a number of regions of interest of the right hemisphere. Abbreviations: Anterior cingulate gyms (AcGg), Superior frontal gyrus (SFG), Precentral (PrG), Postcentral gyrus (PoG), Substantia nigra pars compacta (SNc).

### DETAILED DESCRIPTION

The inventors have identified new therapeutic strategies to treat tauopathies by gene therapy, and more specifically Alzheimer's Disease, in particular sporadic Alzheimer's Disease.

The disclosure therefore relates to a viral particle, and its use in treating tauopathy by gene therapy in a subject in need thereof, said viral particle comprising a viral vector or a nucleic acid construct including a transgene encoding a glucocerebrosidase.

As used herein, the term “viral particle” relates to an infectious and typically replication-defective virus particle comprising (i) a viral vector packaged within (ii) a capsid and, as the case may be, (iii) a lipidic envelope surrounding the capsid.

The term “viral vector” typically refers to the nucleic acid part of the viral particle as disclosed herein, which is packaged in a capsid.

Said viral vector thus typically comprises at least (i) a nucleic acid construct including a transgene and suitable nucleic acid elements for its expression in a host treated by gene therapy, and (ii) all or a portion of a viral genome, for example at least inverted terminal repeats of a viral genome.

As used herein, the term “nucleic acid construct” refers to a non-naturally occurring nucleic acid resulting from the use of recombinant DNA technology. Especially, a nucleic acid construct is a nucleic acid molecule which has been modified to contain segments of nucleic acid sequences, which are combined or juxtaposed in a manner which would not otherwise exist in nature.

As used herein, the term “transgene” refers to nucleic acid molecule, DNA or cDNA encoding a gene product for use as the active principle in gene therapy. The gene product may be an RNA, peptide or protein.

The terms “nucleic acid” and “polynucleotide” or “nucleotide sequence” may be used interchangeably to refer to any molecule composed of or comprising monomeric nucleotides. A nucleic acid may be an oligonucleotide or a polynucleotide. A nucleotide sequence may be a DNA or RNA. A nucleotide sequence may be chemically modified or artificial. Nucleotide sequences include peptide nucleic acids (PNA), morpholines and locked nucleic acids (LNA), as well as glycol nucleic acids (GNA) and threose nucleic acid (TNA). Each of these sequences is distinguished from naturally-occurring DNA or RNA by changes to the backbone of the molecule. Also, phosphorothioate nucleotides may be used. Other deoxynucleotide analogs include methylphosphonates, phosphoramidates, phosphorodithioates, N3′P5′-phosphoramidates and oligoribonucleotide phosphorothioates and their 2′-0-allyl analogs and 2′-0-methylribonucleotide methylphosphonates which may be used in a nucleotide of the invention.

As used herein the term “inverted terminal repeat (ITR)” refers to a nucleotide sequence located at the 5′-end (5′ITR) and a nucleotide sequence located at the 3′-end (3′ITR) of a virus, that contain palindromic sequences and that can fold over to form T-shaped hairpin structures that function as primers during initiation of DNA replication. They are also needed for viral genome integration into the host genome; for the rescue from the host genome; and for the encapsidation of viral nucleic acid into mature virions. The ITRs are required in cis for the vector genome replication and its packaging into the viral particles.

As used here, the term “comprising” does not exclude other elements. For the purposes of the present disclosure, the term “consisting of” is considered to be a preferred embodiment of the term “comprising of”.

As used here, the term “notably”, “typically” or “particularly” are used interchangeably to refer to one alternative among several embodiments and the term “preferably” refers to a preferred embodiment.

As used here SNc is the acronym of substantia nigra pars compacta (SNc).

### The Nucleic Acid Constructs of the Present Disclosure

The nucleic acid construct according to the present disclosure include a transgene and at least suitable nucleic acid elements for its expression in said host treated by gene therapy with the viral vector of the disclosure.

For example, said nucleic acid construct comprises a transgene consisting of the coding sequence of glucocerebrosidase and one or more control sequence required for expression of said coding sequence in the relevant cell types or tissue. Generally, the nucleic acid construct comprises a coding sequence and regulatory sequences preceding (5′ non-coding sequences) and following (3′ non-coding sequences) the coding sequence that are required for expression of the selected gene product. Thus, in specific embodiments, said nucleic acid construct comprises at least (i) a transgene encoding a glucocerebrosidase under the control of (ii) a promoter and (iii) a 3′ untranslated region that usually contains a polyadenylation site and/or transcription terminator. The nucleic acid construct may also comprise additional regulatory elements such as, for example, enhancer sequences, introns, microRNA targeted sequence, a polylinker sequence facilitating the insertion of a DNA fragment within a vector and/or splicing signal sequences.

The specific nucleic acid constructs comprising a nucleic acid sequence selected from the group consisting of: SEQ ID NO: 1, 7, 11, 12 and 19 or a portion of a nucleic acid sequence selected from the group consisting of: SEQ ID NO: 1, 7, 11, 12 and 19 as disclosed hereafter and vectors or particles comprising such specific nucleic acid constructs are also part of the present disclosure.

### The Transgene Encoding Glucocerebrosidase

In particular, the nucleic acid construct according to the present disclosure comprises a transgene encoding glucocerebrosidase, preferably encoding human glucocerebrosidase selected from the group consisting of SEQ ID NO: 5, 6, 8, 17 and 18, preferably encoding human glucocerebrosidase isoform 1 of SEQ ID NO: 5, 6 or 8.

As used herein, the term “glucocerebrosidase” refers to β-Glucocerebrosidase (also called acid β-glucosidase, D-glucosyl-N-acylsphingosine glucohydrolase, or GCase), an enzyme with glucosylceramidase activity (EC 3.2.1.45) that is needed to cleave, by hydrolysis, the beta-glucosidic linkage of the chemical glucocerebroside, an intermediate in glycolipid metabolism that is abundant in cell membranes (particularly skin cells). The term “glucocerebrosidase” refers to the enzyme and any additional co-translation or post-translational modifications.

Human glucocerebrosidase is naturally encoded by GBA1 gene in human that generated five alternatively spliced mRNAs which encode three distinct isoforms of glucocerebrosidase (isoform 1 (SEQ ID NO: 5), isoform 2 (SEQ ID NO: 17) and isoform 3 (SEQ ID NO: 18)). As used herein, the term “glucocerebrosidase” refers to the three isoforms of glucocerebrosidase. Nucleotide sequence corresponding to the coding sequence portion CDS of human GBA1 mRNA isoform 1 (GeneBank ref. M19285.1:123-1733) is represented by SEQ ID NO: 7.

In specific embodiments, said nucleic acid construct comprises all or a portion (at least 1000, 1100, 1500, 2000, 2500 or at least 1500 nucleotides) of a coding nucleic acid sequence having at least 70%, 80%, 90%; 95%, 99% or 100% identity to the coding sequence of a naturally-occurring or recombinant glucocerebrosidase. Naturally occurring glucocerebrosidases include human, primate, murine or other mammalian known glucocerebrosidases, typically human glucocerebrosidase of SEQ ID NO: 5, 17 or 18.

Examples of recombinant glucocerebrosidase include imiglucerase (Cerezyme), velaglucerase (Vpriv) and taliglucerase (Elelyso).

In a preferred embodiment, said nucleic acid construct comprises a transgene encoding glucocerebrosidase selected from the group consisting of SEQ ID NO: 5, 6, 8, 17 and 18, for example a coding sequence as represented by a sequence selected from the group consisting of SEQ ID NO: 1, 7, 11, 12 and 19 or a variant transgene consisting of coding sequence having at least 75%, at least 80% or at least 90% identity to a sequence selected from the group consisting of: SEQ ID NO: 1, 7, 11, 12 and 19. Preferably, said transgene includes a coding nucleic acid portion of a sequence selected from the group consisting of: SEQ ID NO: 1, 7, 11, 12 and 19, e.g. the optimized sequence SEQ ID NO: 1, region 58 . . . 1551 of SEQ ID NO: 7 or 19, region 58 . . . 1611 of SEQ ID NO: 7 or 19 and region 118 . . . 1611 of SEQ ID NO: 7 or 19. In one embodiment, said variant transgene encoding a portion of SEQ ID NO: 5, 6, 8, 17 or 18 or consisting of coding sequence having at least 75%, at least 80% or at least 90% identity to a sequence selected from the group consisting of: SEQ ID NO: 1, 7, 11, 12 and 19 that has substantially the same glucocerebrosidase activity as human glucocerebrosidase. In particular, a variant nucleic acid construct encodes a truncated glucocerebrosidase where one or more of the amino acid residues have been deleted.

As used herein, the term “sequence identity” or “identity” refers to the number of matches (identical nucleic acid or amino acid residues) in positions from an alignment of two polynucleotide or polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, 1970, J Mol Biol.; 48(3):443-53) which aligns the sequences optimally over the entire length, while sequences of substantially different lengths are preferably aligned using a local alignment algorithm (e.g. Smith and Waterman algorithm (Smith and Waterman, 1981, J Theor Biol.; 91(2):379-80) or Altschul algorithm (Altschul S F et al., 1997, Nucleic Acids Res.; 25(17):3389-402.; Altschul S F et al., 2005, Bioinformatics.; 21(8):1451-6). Alignment for purposes of determining percent nucleic acid sequence identity or amino acid sequence identity can be achieved in various ways that are within the skill in the art, for instance, using publicly available computer software available on internet web sites such as http://blast.ncbi.nlm.nih.gov/ or http://www.ebi.ac.uk/Tools/emboss/. Those skilled in the art can determine appropriate parameters for measuring alignment, including any algorithms needed to achieve maximal alignment over the full length of the sequences being compared. For purposes herein, % nucleic acid or amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

The Promoter for Use with the Nucleic Acid Constructs of the Disclosure

In one embodiment, the nucleic acid construct comprises a promoter. Said promoter initiates transgene expression upon introduction into a host cell.

As used herein, the term “promoter” refers to a regulatory element that directs the transcription of a nucleic acid to which it is operably linked. A promoter can regulate both rate and efficiency of transcription of an operably linked nucleic acid. A promoter may also be operably linked to other regulatory elements which enhance (“enhancers”) or repress (“repressors”) promoter-dependent transcription of a nucleic acid. These regulatory elements include, without limitation, transcription factor binding sites, repressor and activator protein binding sites, and any other sequences of nucleotides known to one of skill in the art to act directly or indirectly to regulate the amount of transcription from the promoter, including e.g. attenuators, enhancers, and silencers. The promoter is located near the transcription start site of the gene or coding sequence to which is operably linked, on the same strand and upstream of the DNA sequence (towards the 5′ region of the sense strand). A promoter can be about 100-1000 base pairs long. Positions in a promoter are designated relative to the transcriptional start site for a particular gene (i.e., positions upstream are negative numbers counting back from −1, for example −100 is a position 100 base pairs upstream).

As used herein, the term “operably linked” refers to a linkage of polynucleotide (or polypeptide) elements in a functional relationship. A nucleic acid is “operably linked” when it is placed into a functional relationship with another nucleic acid sequence. For instance, a promoter or transcription regulatory sequence is operably linked to a coding sequence if it affects the transcription of the coding sequence. Operably linked means that the DNA sequences being linked are typically contiguous; where it is necessary to join two protein encoding regions, they are contiguous and in reading frame.

In a particular embodiment, the nucleic acid construct of the disclosure further comprises a promoter operably-linked to the transgene encoding glucocerebrosidase and wherein said promoter directs the expression of said transgene at least in neurons and glial cells typically neuronal and glial cells located in the cerebral cortex and subcellular structures such as the nucleus basalis of Meynert, the substantia nigra pars compacta, the locus ceruleus, the hippocampal formation and the entorhinal cortex, more preferably dopaminergic neurons and microglial cells of the substantia nigra pars compacta (SNc), and preferably also in neuronal cells of other brain areas, including at least the substantia nigra pars compacta, cerebral cortex, amygdala, and caudal intralaminar nuclei of the thalamus.

Typically, such promoter may be tissue or cell type specific promoter, or an organ-specific promoter, or a promoter specific to multiple organs or a systemic or ubiquitous promoter.

As used herein, the term “ubiquitous promoter” more specifically relates to a promoter that is active in a variety of distinct cells or tissues of the brain, for example in both the neurons and glial cells, typically neuronal and glial cells located in the cerebral cortex and subcellular structures such as the nucleus basalis of Meynert, the substantia nigra pars compacta, the locus ceruleus, the hippocampal formation and the entorhinal cortex, more specifically at least the dopaminergic neurons and microglial cells of the substantia nigra pars compacta, and preferably also in neuronal cells of other brain areas, including at least the substantia nigra pars compacta, cerebral cortex, amygdala, and caudal intralaminar nuclei of the thalamus.

Examples of promoter suitable for expression of the transgene in at least neuronal and glial cells, preferably microglial cells of the substantia nigra compacta include without limitation CMV promoter (Kaplitt 1994, Nat. Genet. 8:148-154), SV40 promoter (Hamer 1979, Cell 17:725-735), chicken beta actin (CBA) promoter (Miyazaki 1989, Gene 79:269-277), the CAG promoter (Niwa 1991, Gene 108:193-199), the b-glucuronidase promoter (GusB) (Shipley 1991, Genetics 10:1009-1018), the Elongation factor 1 alpha promoter (EF1α) (Nakai 1998, Blood 91:4600-4607), the human synapsin 1 gene promoter (hSyn) (Kugler S. et al. Gene Ther. 2003. 10(4):337-47) or the phosphoglycerate kinase 1 promoter (PGK1) (Hannan 1993, Gene 130:233-239).

In a particular embodiment, said ubiquitous promoter can be selected from the group consisting of: human ubiquitin C (UbC) promoter, preferably of SEQ ID NO: 22 or 23 or 28, human Phosphoglycerate Kinase 1 (PGK) promoter, preferably of SEQ ID NO: 24 or 29 and human CBA/CBh promoter of SEQ ID NO: 25 or 26 or 30.

In one embodiment, the promoter is the GusB gene promoter, typically of SEQ ID NO: 2 or 20. In another embodiment, the promoter is the CAG promoter, typically of SEQ ID NO: 9 or 21. In another embodiment, the promoter is hSyn 1 promoter, typically SEQ ID: 13.

All these promoter sequences have properties of allowing expression of said transgene in at least neuronal and glial cells, typically neuronal and glial cells located in the cerebral cortex and subcellular structures such as the nucleus basalis of Meynert, the substantia nigra pars compacta, the locus ceruleus, the hippocampal formation and the entorhinal cortex, more specifically at least the dopaminergic neurons and microglial cells of the substantia nigra pars compacta, and preferably also in neuronal cells of other brain areas, including at least the substantia nigra pars compacta, cerebral cortex, amygdala, and caudal intralaminar nuclei of the thalamus.

In a preferred embodiment, said nucleic acid construct includes the GusB promoter of SEQ ID NO:2 or 20 operably linked to a transgene encoding a glucocerebrosidase, typically selected from the group consisting of SEQ ID NO:1, 7, 11, 12 and 19. In another embodiment, said nucleic acid construct includes the CAG promoter of SEQ ID NO: 9 or 21 operably linked to a transgene encoding a glucocerebrosidase, typically selected from the group consisting of SEQ ID NO: 1, 7, 11, 12 and 19. In another embodiment, said nucleic acid construct includes the hSyn promoter of SEQ ID NO: 13 operably linked to a transgene encoding a glucocerebrosidase, typically selected from the group consisting of SEQ ID NO: 1, 7, 11, 12 and 19.

In specific embodiments, the promoter for use in the present disclosure may be a chemical inducible promoter. As used herein, a chemical inducible promoter is a promoter that is regulated by the in vivo administration of a chemical inducer to said subject in need thereof. Examples of suitable chemical inducible promoters include without limitation Tetracycline/Minocycline inducible promoter (Chtarto 2003, Neurosci Lett. 352:155-158) or rapamycin inducible systems (Sanftner 2006, Mol Ther. 13:167-174).

The Polyadenylation Sequence for Use with the Nucleic Acid Constructs of the Disclosure

Each of these nucleic acid construct embodiments may also include a polyadenylation signal sequence; together or not with other optional nucleotide elements. As used herein, the term “polyadenylation signal” or “poly(A) signal” refers to a specific recognition sequence within 3′ untranslated region (3′ UTR) of the gene, which is transcribed into precursor mRNA molecule and guides the termination of the gene transcription. Poly(A) signal acts as a signal for the endonucleolytic cleavage of the newly formed precursor mRNA at its 3′-end, and for the addition to this 3′-end of a RNA stretch consisting only of adenine bases (polyadenylation process; poly(A) tail). Poly(A) tail is important for the nuclear export, translation, and stability of mRNA. In the context of the invention, the polyadenylation signal is a recognition sequence that can direct polyadenylation of mammalian genes and/or viral genes, in mammalian cells.

Poly(A) signals typically consist of a) a consensus sequence AAUAAA, which has been shown to be required for both 3′-end cleavage and polyadenylation of premessenger RNA (pre-mRNA) as well as to promote downstream transcriptional termination, and b) additional elements upstream and downstream of AAUAAA that control the efficiency of utilization of AAUAAA as a poly(A) signal. There is considerable variability in these motifs in mammalian genes.

In one embodiment, optionally in combination with one or more features of the various embodiments described above or below, the polyadenylation signal sequence of the nucleic acid construct of the invention is a polyadenylation signal sequence of a mammalian gene or a viral gene. Suitable polyadenylation signals include, among others, a SV40 early polyadenylation signal, a SV40 late polyadenylation signal, a HSV thymidine kinase polyadenylation signal, a protamine gene polyadenylation signal, an adenovirus 5 EIb polyadenylation signal, a growth hormone polydenylation signal, a PBGD polyadenylation signal, in silico designed polyadenylation signal (synthetic) and the like.

In a particular embodiment, the polyadenylation signal sequence of the nucleic acid construct is a polyadenylation signal sequence based on bovine growth hormone gene, more particularly, the polyadenylation signal of SEQ ID NO: 3.

In specific embodiments, the nucleic acid construct for use according to the present disclosure includes the GusB promoter of SEQ ID NO:2 or 20 operably linked to the coding sequence of GBA1 gene selected from the group consisting of SEQ ID NO:1, 7, 11, 12 and 19 and the polyadenylation signal sequence of SEQ ID NO:3.

In specific embodiments, the nucleic acid construct for use according to the present disclosure includes the CAG promoter of SEQ ID NO:9 or 21 operably linked to the coding sequence of GBA1 gene selected from the group consisting of SEQ ID NO: 1, 7, 11, 12 and 19 and the polyadenylation signal sequence of SEQ ID NO:3.

In specific embodiments, the nucleic acid construct for use according to the present disclosure includes the hSyn 1 promoter of SEQ ID NO:13 operably linked to the coding sequence of GBA1 gene selected from the group consisting of SEQ ID NO: 1, 7, 11, 12 and 19 and the polyadenylation signal sequence of SEQ ID NO:3.

### Viral Vector

Viral vectors of the present disclosure typically comprise at least (i) a nucleic acid construct including a transgene and suitable nucleic acid elements for its expression in said host treated by gene therapy, and (ii) all or a portion of a viral genome, for example at least inverted terminal repeats of a viral genome.

In one embodiment, the viral vector according to the present disclosure comprises a 5′ITR, and a 3′ITR of a virus, and, optionally a w packaging signal.

“ψ packaging signal” is a cis-acting nucleotide sequence of the virus genome, which in some viruses (e.g. adenoviruses, lentiviruses . . . ) is essential for the process of packaging the virus genome into the viral capsid during replication.

In one embodiment, the viral vector comprises a 5′ITR and a 3′ITR of a virus selected from the group consisting of parvoviruses (in particular adeno-associated viruses), adenoviruses, alphaviruses, retroviruses (in particular gamma retroviruses, and lentiviruses), herpesviruses, and SV40; in a preferred embodiment the virus is an adeno-associated virus (AAV), an adenovirus (Ad), or a lentivirus.

In one embodiment, the viral vector comprises a 5′ITR and a 3′ITR of an AAV.

AAV has arisen considerable interest as a potential vector for human gene therapy. Among the favourable properties of the virus are its lack of association with any human disease, its ability to infect both dividing and non-dividing cells, and the wide range of cell lines derived from different tissues that can be infected. The AAV genome is composed of a linear, single-stranded DNA molecule which contains 4681 bases (Berns and Bohenzky, 1987, Advances in Virus Research (Academic Press, Inc.) 32:243-307). The genome includes inverted terminal repeats (ITRs) at each end, which function in cis as origins of DNA replication and as packaging signals for the virus. The ITRs are approximately 145 bp in length.

AAV ITRs in the viral vectors of the invention may have a wild-type nucleotide sequence or may be altered by the insertion, deletion or substitution of one or more nucleotides, typically, no more than 5, 4, 3, 2 or 1 nucleotide insertion, deletion or substitution as compared to known AAV ITRs. The serotype of the inverted terminal repeats (ITRs) of the AAV vector may be selected from any known human or non-human AAV serotype.

In specific embodiments, the viral vector may be carried out by using ITRs of any AAV serotype. Known AAV ITRs include without limitations, AAV1, AAV2, AAV3 (including types 3A and 3B), AAV4, AAV5, AAV6, AAV7, AAV8, AAV9, AAV10, AAV11, AAV12, avian AAV, bovine AAV, canine AAV, equine AAV, ovine AAV.

In one embodiment, the nucleic acid construct described above is comprised in said viral vector which further comprises a 5′ITR and a 3′ITR of an AAV of a serotype AAV2. In a particular embodiment, the viral vector comprises a 5′ITR and 3′ITR of an AAV of a serotype AAV2, preferably of SEQ ID NO: 15 and/or 16 or a sequence having at least 80% or at least 90% of identity with SEQ ID NO: 15 and/or 16.

Hence, in a more specific embodiment, the viral vector of the disclosure, includes a nucleic acid construct including a GusB promoter of SEQ ID NO: 2 or 20 operably linked to the coding sequence of glucocerebrosidase selected from the group consisting of SEQ ID NO: 1, 7, 11, 12 and 19 and said viral vector further includes AAV ITRs flanking said nucleic acid construct, such as 5′ and 3′ ITRs of AAV2, preferably of SEQ ID NO: 15 and/or 16 or a sequence having at least 80% or at least 90% of identity with SEQ ID NO: 15 and/or 16.

In another specific embodiment, the viral vector of the disclosure, includes a nucleic acid construct including promoter selected from the group consisting of a CAG gene promoter of SEQ ID NO: 9 or 21 operably linked to the coding sequence of glucocerebrosidase selected from the group consisting of SEQ ID NO: 1, 7, 11, 12 and 19 and said viral vector further includes AAV ITRs flanking said nucleic acid construct, such as 5′ and 3′ ITRs of AAV2, preferably of SEQ ID NO: 15 and/or 16 or a sequence having at least 80% or at least 90% of identity with SEQ ID NO: 15 and/or 16.

In another specific embodiment, the viral vector of the disclosure, includes a nucleic acid construct including promoter selected from the group consisting of a hSyn 1 gene promoter of SEQ ID NO: 13 operably linked to the coding sequence of glucocerebrosidase selected from the group consisting of SEQ ID NO: 1, 7, 11, 12 and 19 and said viral vector further includes AAV ITRs flanking said nucleic acid construct, such as 5′ and 3′ ITRs of AAV2, preferably of SEQ ID NO: 15 and/or 16 or a sequence having at least 80% or at least 90% of identity with SEQ ID NO: 15 and/or 16.

In a particular embodiment, the viral vector of the disclosure comprises or consists of SEQ ID NO: 4 or a sequence having at least 80% or at least 90% of identity with SEQ ID NO: 4.

On the other hand, the viral vector of the disclosure can be carried out by using synthetic 5′ITR and/or 3′ITR; and also by using a 5′ITR and a 3′ITR which come from viruses of different serotype. All other viral genes required for viral vector replication can be provided in trans within the virus-producing cells (packaging cells) as described below. Therefore, their inclusion in the viral vector is optional.

In one embodiment, the viral vector comprises a 5′ITR, a w packaging signal, and a 3′ITR of a virus.

### Viral Particle

The viral vector as disclosed above may be packaged in a capsid formed by the capsid proteins, thereby constituting a viral particle as described in the next section.

In preferred embodiments, the capsid is formed of capsid proteins of adeno-associated virus, hereafter referred as an AAV vector particle.

As used herein, an AAV vector particle comprises at least 5′ITR and 3′ITR of an AAV genome and capsid proteins of adeno-associated virus. The term AAV vector particle encompasses any recombinant AAV vector particle (rAAV) or mutant AAV vector particle obtained by genetic engineering of known rAAV.

Proteins of the viral capsid of an adeno-associated virus include the capsid proteins VP1, VP2, and VP3. Differences among the capsid protein sequences of the various AAV serotypes result in the use of different cell surface receptors for cell entry. In combination with alternative intracellular processing pathways, this gives rise to distinct tissue tropisms for each AAV serotype.

In a particular embodiment, an AAV viral particle according to the disclosure may be prepared by encapsulating the viral vector of an AAV vector/genome derived from a particular AAV serotype on a viral particle formed by natural Cap proteins corresponding to an AAV of the same particular serotype. Nevertheless, several techniques have been developed to modify and improve the structural and functional properties of naturally occurring AAV viral particles (Bünning H et al. J Gene Med 2008; 10: 717-733). Thus, in another embodiment, AAV viral particles according to the disclosure includes the nucleic acid construct including the gene encoding glucocerebrosidase as flanked by ITR(s) of a given AAV serotype packaged, for example, into: a) a viral particle constituted of capsid proteins derived from the same or different AAV serotype [e.g. AAV2 ITRs and AAV9 capsid proteins; AAV2 ITRs and AAV TT capsid proteins or other capsid proteins from AAVretro serotypes such as AAV2-retro, AAVMNM004 or AAVMNM008; etc]; b) a mosaic viral particle constituted of a mixture of capsid proteins from different AAV serotypes or mutants [e.g. AAV2 ITRs with a capsid formed by proteins of two or multiple AAV serotypes]; c) a chimeric viral particle constituted of capsid proteins that have been truncated by domain swapping between different AAV serotypes or variants [e.g. AAV2 ITRs with AAV5 capsid proteins with AAV3 domains]; or d) a targeted viral particle engineered to display selective binding domains, enabling stringent interaction with target cell specific receptors.

AAV-based gene therapy targeting the CNS have already been reviewed in Pignataro D, Sucunza D, Rico A J et al., J Neural Transm 2018; 125:575-589. More specifically, the AAV particles may be selected and/or engineered to target at least neuronal and glial cells, and in particular at least neurons and glial cells located in the cerebral cortex and subcellular structures such as the nucleus basalis of Meynert, the substantia nigra pars compacta, the locus ceruleus, the hippocampal formation and the entorhinal cortex, more specifically at least the dopaminergic neurons and microglial cells of the substantia nigra pars compacta.

In specific embodiments, examples of AAV serotype of the capsid proteins for use of AAV viral particle according to the present disclosure include AAV2, AAV5, AAV9, AAV2-retro, AAV MNM004, AAV MNM008, and AAV TT. In more preferred embodiments, said AAV serotype of the capsid proteins are selected from AAV9 and AAV TT serotype.

In a particular embodiment, optionally in combination with one or more features of the various embodiments described above or below, the viral particle is a recombinant AAV viral particle comprising a AAV viral vector as described above, preferably including a coding sequence selected from the group consisting of SEQ ID NO: 1, 7, 11, 12 and 19, and comprising capsid proteins of an AAV9 serotype or of an AAV TT serotype, preferably capsid protein of AAV TT serotype which comprises amino acid sequence SEQ ID NO: 14 or an amino acid sequence having at least 98.5%, preferably 99% or 99.5% of identity with SEQ ID NO: 14.

In another specific embodiment, the viral particle comprises a nucleic acid construct including a coding sequence of human glucocerebrosidase under the control of a promoter, said promoter allowing expression of said human glucocerebrosidase in at least both neurons and glial cells, preferably both neurons and microglial cells. and said viral particle is selected among viral particles that targets at least neurons and glial cells, preferably at least neurons and microglial cells of the substantia nigra pars compacta, typically AAV particles including capsid proteins selected from the group consisting of AAV2, AAV5, AAV9, AAV MNM004, AAV MNM008 or AAV TT serotypes, preferably capsid protein of AAV TT serotype which comprises amino acid sequence SEQ ID NO: 14 or an amino acid sequence having at least 98.5%, preferably 99% or 99.5% of identity with SEQ ID NO: 14.

In a more specific embodiment, such recombinant AAV viral particle according to the present disclosure includes capsid proteins of the AAV9, AAV MNM004, AAV MNM008 or AAV TT serotype and a AAV viral vector including (i) a nucleic acid construct comprising a promoter selected from the group consisting of: GusB promoter of SEQ ID NO: 2 or 20, a CAG promoter of SEQ ID NO: 9 or 21 and hSyn promoter of SEQ ID NO: 13 operably linked to a coding sequence of glucocerebrosidase selected from the group consisting of SEQ ID NO: 1, 7, 11, 12 and 19 and (ii) AAV ITRs, such as 5′ and 3′ ITRs of AAV2, flanking said nucleic acid construct, preferably 5′ and 3′ ITRs of SEQ ID NO: 15 and/or 16 or a sequence having at least 80% or at least 90% of identity with SEQ ID NO: 15 and/or 16.

In a more specific embodiment, such recombinant AAV viral particle according to the present disclosure includes capsid proteins of the AAV TT serotype which comprises amino acid sequence SEQ ID NO: 14 or an amino acid sequence having at least 98.5%, preferably 99% or 99.5% of identity with SEQ ID NO: 14 and a AAV viral vector including (i) a nucleic acid construct comprising a GusB promoter of SEQ ID NO: 2 or 20 operably linked to a coding sequence of glucocerebrosidase selected from the group consisting of SEQ ID NO:1, 7, 11, 12 and 19 and (ii) AAV ITRs, such as 5′ and 3′ ITRs of AAV2, flanking said nucleic acid construct, preferably 5′ and 3′ ITRs of SEQ ID NO: 15 and/or 16 or a sequence having at least 80% or at least 90% of identity with SEQ ID NO: 15 and/or 16.

In a more specific embodiment, such recombinant AAV viral particle according to the present disclosure includes capsid proteins of the AAV TT serotype which comprises amino acid sequence SEQ ID NO: 14 or an amino acid sequence having at least 98.5%, preferably 99% or 99.5% of identity with SEQ ID NO: 14 and a AAV viral vector including (i) a nucleic acid construct comprising a CAG promoter of SEQ ID NO: 9 or 21 operably linked to a coding sequence of glucocerebrosidase selected from the group consisting of SEQ ID NO:1, 7, 11, 12 and 19 and (ii) AAV ITRs, such as 5′ and 3′ ITRs of AAV2, flanking said nucleic acid construct, preferably 5′ and 3′ ITRs of SEQ ID NO: 15 and/or 16 or a sequence having at least 80% or at least 90% of identity with SEQ ID NO: 15 and/or 16.

In a more specific embodiment, such recombinant AAV viral particle according to the present disclosure include capsid proteins of the AAV TT serotype which comprises amino acid sequence SEQ ID NO: 14 or an amino acid sequence having at least 98.5%, preferably 99% or 99.5% of identity with SEQ ID NO: 14 and a AAV viral vector including (i) a nucleic acid construct comprising hSyn promoter of SEQ ID NO: 13 operably linked to a coding sequence of glucocerebrosidase selected from the group consisting of SEQ ID NO:1, 7, 11, 12 and 19 and (ii) AAV ITRs, such as 5′ and 3′ ITRs of AAV2, flanking said nucleic acid construct, preferably 5′ and 3′ ITRs of SEQ ID NO: 15 and/or 16 or a sequence having at least 80% or at least 90% of identity with SEQ ID NO: 15 and/or 16.

The construction of recombinant AAV viral particles is generally known in the art and has been described for instance in U.S. Pat. Nos. 5,173,414 and 5,139,941; WO 92/01070, WO 93/03769, Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; and Kotin, R. M. (1994) Human Gene Therapy 5:793-801. Viral particles with capsid proteins of the serotype AAV TT have also been described in Tordo J, et al., Brain 2018; 141:2014-2031.

Viral Particle with Retrograde Transport

In some embodiments, said viral particle according to the present disclosure is selected among viral variant serotypes with retrograde transport (AAVretro).

Axonal transport (sometimes also called axoplasmic transport or axoplasmic flow) refers to the movement of cellular organelles and proteins from the cell body of a given neuron toward the axon terminal endings (known as anterograde transport). As used herein, the term “retrograde transport” refers to the transport of particles in the opposite direction, i.e. from the axon terminals back to the parent cell bodies. In this regard, neurotropic viruses (rabies viruses being best example) are typically taken up by axon terminals and transported to the neuron's cell body by taking advantage of retrograde transport.

Examples of AAVretro particles includes without limitation capsid protein, preferably capsid protein of AAV2-retro, AAV-TT, AAV-MNM004 and AAV-MNM008, more preferably VP1 capsid protein of AAV2-retro, AAV-TT, AAV-MNM004 and AAV-MNM008.

AAV2-retro capsid protein has been described in WO2017/218842A1.

Other variegated different types of modified viral capsids such as AAV-TT, AAV-MNM004 and AAV-MNM008, have also been designed to transduce neurons innervating the area where the viral vector is delivered through the retrograde spread of the viral vector.

AAV-MNM004 and AAV-MNM008 are described for example in Davidsson et al. Proc. Natl. Acad. Sci. U.S.A. Dec. 9 2019 doi: 10.1073/pnas.1910061116 and in WO2019/158619.

AAV-TT capsid also named AAV2 true-type capsid is described for example in WO2015/121501. In one embodiment, AAV-TT VP1 capsid protein comprises at least one amino acid substitution with respect to the wild type AAV VP1 capsid protein at a position corresponding to one or more of the following positions in an AAV2 protein sequence (NCBI Reference sequence: YP_680426.1): 125, 151, 162, 312, 457, 492, 499, 533, 546, 548, 585, 588 and/or 593, more particularly, AAV-TT comprises one or more of the following amino acid substitutions with respect to a wild type AAV2 VP1 capsid protein (NCBI Reference sequence: YP_680426.1): V125I, V151A, A162S, T205S, N312S, Q457M, S492A, E499D, F533Y, G546D, E548G, R585S, R588T and/or A593S. In one particular embodiment, AAV-TT comprises four or more mutations with respect to the wild type AAV2 VP1 capsid protein at the positions 457, 492, 499 and 533.

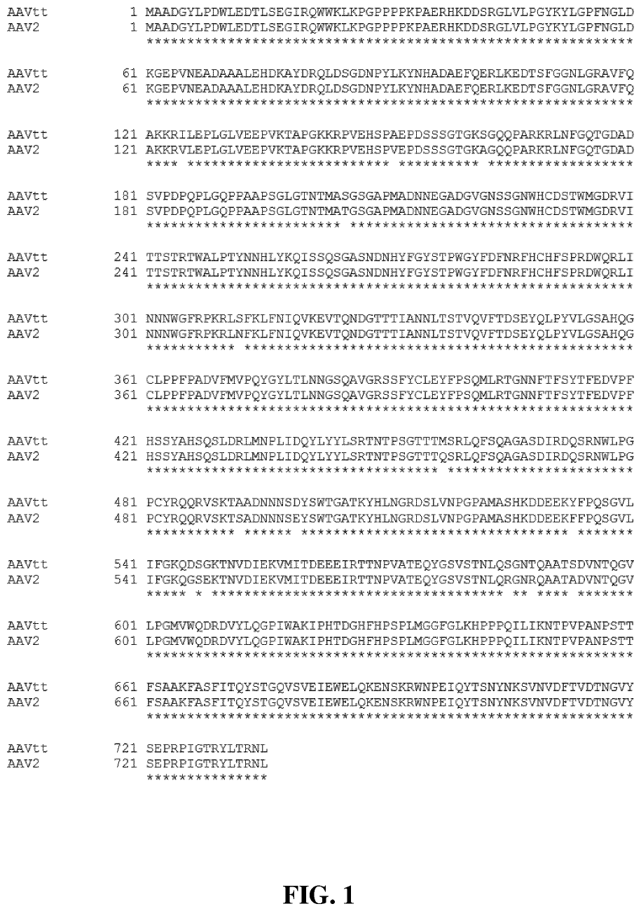

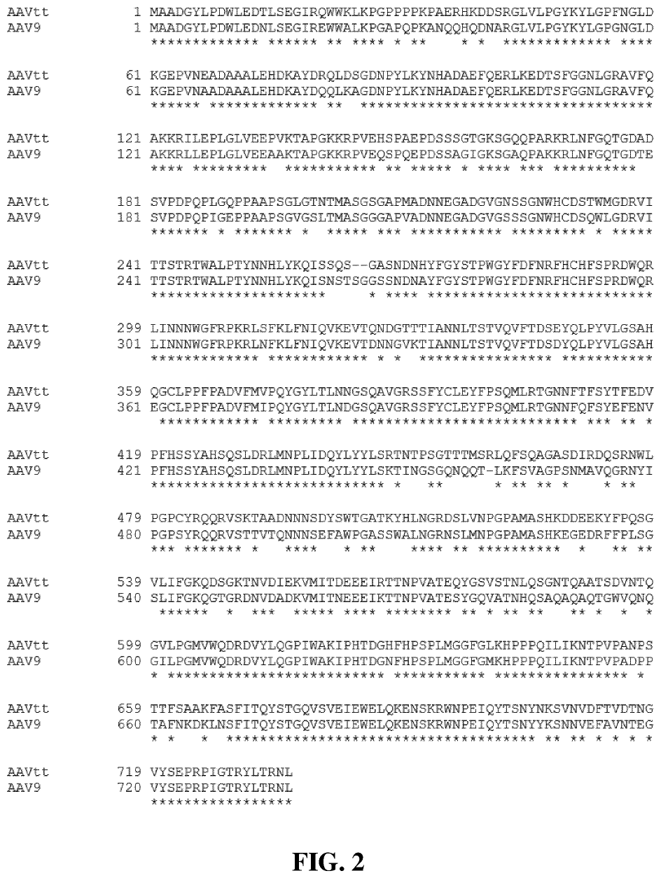

In further embodiments, AAV-TT capsid may be from an AAV serotype other than AAV2 and can be derived for example from AAV1, AAV3B, AAV-LK03, AAV5, AAV6, AAV8, AAV9 or AAV10 capsid protein. In particular, the positions corresponding to those described above with respect to AAV2 can be easily identified by sequence alignments, for example as provided in FIGS. 1 and 2. In one embodiment, AAV-TT VP1 capsid protein of the disclosure comprises or consists of amino acid sequence SEQ ID NO: 14 or an amino acid sequence having at least 98.5%, preferably 99% or 99.5% of identity with SEQ ID NO: 14.

In specific embodiments, said AAVretro viral particles are selected according to the present disclosure among those that are able to retrogradely disseminate in the cerebral cortex, preferably at least to the substantia nigra pars compacta and cerebral cortex after intraparenchymal injection in the caudate or putamen nuclei of non-human primate as determined in an in vivo dissemination assay.

In a more specific embodiment, said AAVretro viral particles according to the present disclosure are selected among those which are able to retrogradely disseminate in the cerebral cortex, preferably at least to substantia nigra pars compacta and cerebral cortex after intraparenchymal injection in the caudate or putamen nuclei of non-human primate to at least the same level as AAV-TT as determined in an in vivo dissemination assay.

The inventors indeed designed an in vivo dissemination assay enabling to determine rAAV with true retrograde transport for their use in gene therapies for treating tauopathies as disclosed herein, such as Alzheimer's disease, and to compare for example with a positive control such as AAV-TT rAAV-GFP.

One important feature of the dissemination assay is that it is an in vivo assay in non-human primate where the rAAV are injected in an area without the presence of fibers of passage. Accordingly, no false positive uptake can be obtained by fibers of passage, i.e. fibers coursing through the injected area towards more distant destination. In non-human primates, the caudate and putamen nuclei are 100% parenchymous structures, and therefore do not contain fibers of passage. Hence, advantageously, suitable rAAV with retrograde transport can be compared and selected according to the present disclosure by means of the proposed dissemination assay.

In a preferred embodiment, said AAV retro viral particle includes AAV TT serotype capsid protein which comprises amino acid sequence SEQ ID NO: 14 or an amino acid sequence having at least 98.5%, preferably 99% or 99.5% of identity with SEQ ID NO: 14 and is able to disseminate retrogradely in the cerebral cortex, preferably at least to the substantia nigra pars compacta and cerebral cortex after intraparenchymal injection in the caudate or putamen nuclei of non-human primate as determined in an in vivo dissemination assay.

In a preferred embodiment, said in vivo dissemination assay includes the following steps:
a. injecting a test rAAV comprising a GFP-encoding transgene (rAAV-GFP) by intraparenchymal injection of said rAAV-GFP into the post-commissural putamen of a non-human primate,
b. Counting the number of GFP-expressing neurons in the cerebral cortex, preferably in the brain areas innervating the caudate putamen nuclei one month post injection.

GFP encoding transgene may be prepared from GFP encoding nucleic acid of SEQ ID NO: 10 or SEQ ID NO: 27 or functional variants thereof with optimized sequence or truncated forms. Neurons expressing GFP may be visualized by immunoperoxidase stains, using anti-GFP antibodies. GFP-expressing neurons may advantageously be automatically counted throughout the cerebral cortex of the injected non-human primates. A preferential location of GFP-positive neurons is expected to occur in deep layers of the cerebral cortex. Besides cortical areas, GFP-expressing neurons may also be quantified in all brain areas innervating the injected post-commissural putamen or caudate-putamen nuclei, particularly at least the substantia nigra pars compacta, the amygdala and the caudal intralaminar nuclei.

In one specific embodiment, an AAV-retro viral particle according to the present disclosure is selected among those where at least 50%, 60%, 70%, 80% or at least 90% of the neurons of the deep layers V-VI of the cerebral cortex innervating the injected site are expressing GFP as determined in said in vivo dissemination assay. In a preferred embodiment, said AAV retro viral particle includes AAV TT serotype capsid protein which comprises amino acid sequence SEQ ID NO: 14 or an amino acid sequence having at least 98.5%, preferably 99% or 99.5% of identity with SEQ ID NO: 14 and where at least 50%, 60%, 70%, 80% or at least 90% of the neurons of the deep layers V-VI of the cerebral cortex innervating the injected site are expressing GFP as determined in said in vivo dissemination assay.

In a more specific embodiment, the dissemination assay is carried out as described in the examples.

In a more specific embodiment, said in vivo dissemination assay includes the following steps:
a. injecting a test rAAV comprising GFP transgene by intraparenchymal injection of said rAAV-GFP into the post-commissural putamen of a non-human primate,
b. counting the number of GFP-expressing neurons in the cerebral cortex, preferably in the brain areas innervating the caudate putamen nuclei, more preferably at least in the cerebral cortex, substantia nigra, amygdala and caudal intralaminar nuclei one month post injection,
c. comparing the percentage of labelled neurons in the cerebral cortex with a control experiment performed with AAV-TT-GFP.

In other embodiments, said AAVretro includes capsid proteins selected among the following variant serotypes: AAV2-retro, AAV-MNM004, AAV-MNM008 and AAV-TT.

In a preferred embodiment, said AAV retro viral particle includes AAV TT serotype capsid protein which comprises amino acid sequence SEQ ID NO: 14 or amino acid sequence having at least 98.5%, preferably 99% or 99.5% of identity with SEQ ID NO: 14.

### A Process for Producing Viral Particles

Production of viral particles carrying the expression viral vector as disclosed above can be performed by means of conventional methods and protocols, which are selected taking into account the structural features chosen for the actual embodiment of the viral particles to be produced.

Briefly, viral particles can be produced in a host cell, more particularly in specific virus-producing cell (packaging cell), which is transfected with the nucleic acid construct or viral vector to be packaged, in the presence of a helper vector or virus or other DNA construct(s).

The term “packaging cells” as used herein, refers to a cell or cell line which may be transfected with a nucleic acid construct or viral vector of the disclosure, and provides in trans all the missing functions which are required for the complete replication and packaging of a viral vector. Typically, the packaging cells express in a constitutive or inducible manner one or more of said missing viral functions. Said packaging cells can be adherent or suspension cells.

Typically, a process of producing viral particles comprises the following steps:
a) culturing a packaging cell comprising a nucleic acid construct or viral vector as described above in a culture medium; and
b) harvesting the viral particles from the cell culture supernatant and/or inside the cells.

Conventional methods can be used to produce viral particles of the AAV viral particles, which consist on transient cell co-transfection with nucleic acid construct or expression vector (e.g. a plasmid) carrying the transgene encoding glucocerebrosidase; a nucleic acid construct (e.g., an AAV helper plasmid) that encodes rep and cap genes, but does not carry ITR sequences; and with a third nucleic acid construct (e.g., a plasmid) providing the adenoviral functions necessary for AAV replication. Viral genes necessary for AAV replication are referred herein as viral helper genes. Typically, said genes necessary for AAV replication are adenoviral helper genes, such as E1A, E1B, E2a, E4, or VA RNAs. Preferably, the adenoviral helper genes are of the Ad5 or Ad2 serotype.

Large-scale production of AAV particles according to the disclosure can also be carried out for example by infection of insect cells with a combination of recombinant baculoviruses (Urabe et al. Hum. Gene Ther. 2002; 13: 1935-1943). SF9 cells are co-infected with two or three baculovirus vectors respectively expressing AAV rep, AAV cap and the AAV vector to be packaged. The recombinant baculovirus vectors will provide the viral helper gene functions required for virus replication and/or packaging. Smith et al 2009 (Molecular Therapy, vol. 17, no. 11, pp 1888-1896) further describes a dual baculovirus expression system for large-scale production of AAV particles in insect cells.

Suitable culture media will be known to a person skilled in the art. The ingredients that compose such media may vary depending on the type of cell to be cultured. In addition to nutrient composition, osmolarity and pH are considered important parameters of culture media. The cell growth medium comprises a number of ingredients well known by the person skilled in the art, including amino acids, vitamins, organic and inorganic salts, sources of carbohydrate, lipids, trace elements (CuS04, FeS04, Fe(N03)3, ZnS04 . . . ), each ingredient being present in an amount which supports the cultivation of a cell in vitro (i.e., survival and growth of cells). Ingredients may also include different auxiliary substances, such as buffer substances (like sodium bicarbonate, Hepes, Tris . . . ), oxidation stabilizers, stabilizers to counteract mechanical stress, protease inhibitors, animal growth factors, plant hydrolyzates, anti-clumping agents, anti-foaming agents. Characteristics and compositions of the cell growth media vary depending on the particular cellular requirements. Examples of commercially available cell growth media are: MEM (Minimum Essential Medium), BME (Basal Medium Eagle) DMEM (Dulbecco's modified Eagle's Medium), Iscoves DMEM (Iscove's modification of Dulbecco's Medium), GMEM, RPMI 1640, Leibovitz L-15, McCoy's, Medium 199, Ham (Ham's Media) F10 and derivatives, Ham F12, DMEM/F12, etc.

Further guidance for the construction and production of viral vectors for use according to the disclosure can be found in Viral Vectors for Gene Therapy, Methods and Protocols. Series: Methods in Molecular Biology, Vol. 737. Merten and Al-Rubeai (Eds.); 2011 Humana Press (Springer); Gene Therapy. M. Giacca. 2010 Springer-Verlag; Heilbronn R. and Weger S. Viral Vectors for Gene Transfer: Current Status of Gene Therapeutics. In: Drug Delivery, Handbook of Experimental Pharmacology 197; M. Schäfer-Korting (Ed.). 2010 Springer-Verlag; pp. 143-170; Adeno-Associated Virus: Methods and Protocols. R. O. Snyder and P. Moulllier (Eds). 2011 Humana Press (Springer); Bunning H. et al. Recent developments in adeno-associated virus technology. J. Gene Med. 2008; 10:717-733; Adenovirus: Methods and Protocols. M. Chinón and A. Bosch (Eds.); Third Edition. 2014 Humana Press (Springer)

The disclosure also relates to a host cell comprising a nucleic acid construct or a viral vector encoding glucocerebrosidase as described above. More particularly, host cell according to the disclosure is a specific virus-producing cell, also named packaging cell which is transfected with the a nucleic acid construct or a viral vector as described above, in the presence of a helper vector or virus or other DNA constructs and provides in trans all the missing functions which are required for the complete replication and packaging of a viral particle. Said packaging cells can be adherent or suspension cells

For example, said packaging cells may be eukaryotic cells such as mammalian cells, including simian, human, dog and rodent cells. Examples of human cells are PER.C6 cells (WO01/38362), MRC-5 (ATCC CCL-171), WI-38 (ATCC CCL-75), HEK-293 cells (ATCC CRL-1573), HeLa cells (ATCC CCL2) and fetal rhesus lung cells (ATCC CL-160). Examples of non-human primate cells are Vero cells (ATCC CCL81), COS-1 cells (ATCC CRL-1650) or COS-7 cells (ATCC CRL-1651). Examples of dog cells are MDCK cells (ATCC CCL-34). Examples of rodent cells are hamster cells, such as BHK21-F, HKCC cells, or CHO cells.

As an alternative to mammalian sources, the packaging cells for producing the viral particles may be derived from avian sources such as chicken, duck, goose, quail or pheasant. Examples of avian cell lines include avian embryonic stem cells (WO01/85938 and WO03/076601), immortalized duck retina cells (WO2005/042728), and avian embryonic stem cell derived cells, including chicken cells (WO2006/108846) or duck cells, such as EB66 cell line (WO2008/129058 & WO2008/142124).

In another embodiment, the cells can be any packaging cells permissive for baculovirus infection and replication. In a particular embodiment, said cells are insect cells, such as SF9 cells (ATCC CRL-1711), Sf21 cells (IPLB-Sf21), MG1 cells (BTI-TN-MG1) or High Five™ cells (BTI-TN-5B1-4).

Accordingly, in a particular embodiment, optionally in combination with one or more features of the various embodiments described above or below, the host cell comprises:
a nucleic acid construct or viral vector comprising a transgene encoding glucocerebrosidase as described above (e.g., the AAV vector),
a nucleic acid construct, for example a plasmid, encoding AAV rep and/or cap genes which does not carry the ITR sequences; and, optionally,
a nucleic acid construct, for example a plasmid or virus, comprising viral helper genes.

In another aspect, the disclosure relates to a host cell transduced with a viral particle of the disclosure and the term “host cell” as used herein refers to any cell line that is susceptible to infection by a virus of interest, and amenable to culture in vitro.

### Pharmaceutical Compositions

Another aspect of the present disclosure relates to a pharmaceutical composition comprising a nucleic acid construct, a viral vector, a viral particle or a host cell of the disclosure in combination with one or more pharmaceutical acceptable excipient, diluent or carrier.

As used herein, the term “pharmaceutically acceptable” means approved by a regulatory agency or recognized pharmacopeia such as European Pharmacopeia, for use in animals and/or humans. The term “excipient” refers to a diluent, adjuvant, carrier, or vehicle with which the therapeutic agent is administered.

Any suitable pharmaceutically acceptable carrier, diluent or excipient can be used in the preparation of a pharmaceutical composition (See e.g., Remington: The Science and Practice of Pharmacy, Alfonso R. Gennaro (Editor) Mack Publishing Company, April 1997). Pharmaceutical compositions are typically sterile and stable under the conditions of manufacture and storage. Pharmaceutical compositions may be formulated as solutions (e.g. saline, dextrose solution, or buffered solution, or other pharmaceutically acceptable sterile fluids), microemulsions, liposomes, or other ordered structure suitable to accommodate a high product concentration (e.g. microparticles or nanoparticles). The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition.

Preferably, said pharmaceutical composition is formulated as a solution, more preferably as an optionally buffered saline solution. Supplementary active compounds can also be incorporated into the pharmaceutical compositions of the invention. Guidance on co-administration of additional therapeutics can for example be found in the Compendium of Pharmaceutical and Specialties (CPS) of the Canadian Pharmacists Association.

In one embodiment, the pharmaceutical composition is a composition suitable for intraparenchymal, intracerebral, intravenous, or intrathecal administration. These pharmaceutical compositions are exemplary only and do not limit the pharmaceutical compositions suitable for other parenteral and non-parenteral administration routes. The pharmaceutical compositions described herein can be packaged in single unit dosage or in multidosage forms.

### Therapeutic Uses

Using animal models of sporadic tauopathies in mice, the inventors surprisingly found that AAV-mediated enhancement of glucocerebrosidase activity induces an extensive clearance of tau aggregates both in the cerebral cortex and striatum.

These results provide strong evidence of a possible therapeutic strategy for treating tauopathies, in particular sporadic tauopathies, and more specifically Alzheimer's Disease in human subject.

Hence, the disclosure relates to a method for treating tauopathy, for example Alzheimer's disease, and more specifically sporadic Alzheimer's disease, in a subject in need thereof, said method comprising administering to said subject a therapeutically effective amount of a viral particle or viral vector as described above.

In a particular embodiment, said method comprises administering to a subject a therapeutically effective amount of a viral particle or viral vector as described above to be delivered to neurons of cerebral cortex, preferably neurons of the deep layers V-VI of the cerebral cortex, preferably at least 10%, 20%, 30%, 40%, 50%, 60%, 70%, 80% or at least 90% of the neurons of the deep layers V-VI of the cerebral cortex innervating the administrated site.

In another particular embodiment, said method comprises administering to a subject a therapeutically effective amount of a viral particle or viral vector as described above to be delivered to neurons of brain areas innervating the injection site, preferably to be delivered to neurons of at least brain areas innervating the caudate-putamen nuclei, i.e. at least substantia nigra pars compacta, cerebral cortex, amygdala and caudal intralaminar nuclei of the thalamus, preferably at least 10%, 20%, 30%, 40%, 50%, 60%, 70%, 80% or at least 90% of these neurons.

In another particular embodiment, said method comprises administering to a subject a therapeutically effective amount of a viral particle or viral vector as described above to be delivered to neurons of brain areas innervating the injection site, preferably to be delivered to neurons of at least brain areas innervating the dentate gyrus of the hippocampal formation, i.e. at least neurons located in layers II and III of the entorhinal cortex, preferably at least 10%, 20%, 30%, 40%, 50%, 60%, 70%, 80% or at least 90% of these neurons.

In a further aspect, the disclosure relates to a nucleic acid construct, viral vector, viral particle, host cell or pharmaceutical composition as described above, for use as a medicament in a subject in need thereof, and more specifically, for use in treating tauopathy, preferably Alzheimer's disease, and more specifically sporadic Alzheimer's Disease in a subject in need thereof.

In another further aspect, the disclosure relates to the use of a nucleic acid construct, viral vector, viral particle, host cell or pharmaceutical composition as described above in the manufacture of a medicament, preferably for treating tauopathy, preferably Alzheimer's disease, and more specifically sporadic Alzheimer's disease.

The term “subject” or “patient” as used herein, refers to mammals. Mammalian species that can benefit from the disclosed methods of treatment include, but are not limited to, humans, non-human primates such as apes, chimpanzees, monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease, typically tau aggregates in tauopathies.

In other embodiments, this term refers to minimizing the spread or worsening of the disease resulting from the administration of one or more therapeutic agents to a subject with such a disease.

As used herein, tauopathies refer to diseases where the neuropathological hallmark is represented by the intracytoplasmic aggregation of tau protein in brain tissues, in particular the aggregation of hyperphosphorylated tau protein in the form of neurofibrillary tangles. In particular, tauopathies include neurodegenerative disorders such as Alzheimer's disease, but also Fronto-temporal lobar degeneration (FTD), Progressive supranuclear palsy (PSP), Corticobasal degeneration (CBD), Tangle predominant dementia (TPD), Guam Parkinson dementia complex, Argyrophilic grain disease (AGD), and Pick's disease (AD unrelated).

As used herein, Alzheimer's disease (AD) refers to a progressive neurodegenerative disorder of the central nervous system of an unknown origin. Within the context of AD, the defining characteristic is cognitive impairment. For most of the diagnosed cases, cognitive impairment is accompanied by mood and behavioural symptoms such as depression, anxiety, irritability, inappropriate behaviour, agitation and psychosis. AD diagnosis is often made by clinical assessment and informant interview. According to the Diagnostic and Statistical Handbook of Mental Disorders (DSM-IV), the following criteria are required for reaching a diagnosis of probable Alzheimer's disease: (1) multiple cognitive deficits, (2) social and labour deterioration, (3) gradual beginning and deterioration and (4) cannot be explained by other causes. The criteria issued in 1984 by the National Institute of Neurological and Communicative Disorders and Stroke (NINCDS) and the Alzheimer's disease and Related Disorders Association (ARRDA) are as follows: (1) two or more areas involved, (2) presence of progressive dementia, (3) absence alteration of consciousness, (4) beginning between 40 and 90 years and (5) cannot be explained by other cause. Furthermore, the prodromal pre-Alzheimer's disease state known as Mild Cognitive Impairment (MCI) is defined as an objective abnormal memory loss for the age and educational level of a subject. Criteria for MCI include: (1) Memory complaints corroborated by a family member, (2) other cognitive functions are normal, (3) normal daily activities, (4) abnormal memory for the age and (5) absence of dementia.

The above method is particularly suitable for treating sporadic tauopathies, in particular Alzheimer's disease, and more specifically sporadic Alzheimer's disease. As used herein, sporadic tauopathies (also referred as idiopathic disorders) refers to tauopathy which is not associated to known particular genetic mutations (familial case). Such known genetic mutations associated to familial tauopathies include a mutation in a gene selected from the group consisting of the amyloid precursor protein (APP) gene on chromosome 21, genes encoding presenilin 1 (PSEN1) on chromosome 14 and presenilin 2 (PSEN2) on chromosome 1.

As used herein a “therapeutically effective amount” refers to an amount effective, at dosages and for periods of time necessary to achieve the desired therapeutic result, such as one or more of the following therapeutic results:
a significant reduction of tau aggregates in neurons in said subject,
a significant neuroprotective effect of neurons,
a significant attenuation of microglia-driven proinflammatory phenomena triggered by tau removal,
a significant blockade of the prion-like trans-neuronal passage of tau.

As used, the “prion-like trans-neuronal passage of tau” refers to the ability of protein for jumping from a neuronal axon terminal into the next neuron being innervated by the tau-expressing axon terminal.

A significant reduction of tau burden in the brain area (e.g. in neurons of the cerebral cortex) may correspond to a reduction of at least 10%, 20%, 30%, 40%, 50%, 60%, 70%, 80%, or at least 90% of tau aggregates in the corresponding brain area (e.g. cerebral cortex), after a minimum period of 4 weeks of treatment.

In some embodiments, a significant neuroprotective effect of neurons in a treated patient may be estimated as at least 10%, at least 20% or at least 30% improved neuronal survival vs untreated patients after a minimal period of 52 weeks (a year) of treatment.

In other specific embodiments, a treatment with a product of the disclosure may inhibit the progression or delay the onset, or reduce the severity of one or more symptoms of tauopathies. For example, a treatment may inhibit the progression, or delay the onset, or reduce the severity of one or more of the following symptoms:
degeneration of neurons (e.g. in the nucleus basalis of Meynert, substantia nigra pars compacta, locus ceruleus, hippocampal formation, entorhinal cortex and cerebral cortical areas overall),
mild to severe cognitive impairment,
mood swings,
depression,
apathy,
confusion about time and place,
hallucinations,
behavioral disturbances,
postural instability,
lack of empathy,
impairments in decision-making,
amnesia, irritability,
delusions, agitation, and
disinterest in daily activities and withdrawal from social interaction, among others.

In one embodiment, an effective amount of the viral particle (or viral vector) as described above is administered to the subject or patient by intraparenchymal, intracerebral, intracerebroventricular (icv), intrathechal, or intravenous route.

Typically, a therapeutically effective amount of said viral vector is preferably administered by intrathecal or intraparenchymal route, the latter preferably to brain areas such as the cerebral cortex and subcellular structures, such as the nucleus basalis of Meynert, the substantia nigra pars compacta, the locus ceruleus, the hippocampal formation and the entorhinal cortex.

In a particular embodiment, a therapeutically amount of said viral vector is administered by intraparenchymal route, preferably to the dentate gyrus of the hippocampal formation to be disseminated at least to the neurons located in layers II and III of the entorhinal cortex through the performant path. As used herein, said performant path refers to an anatomical pathway linking the entorhinal cortex and the dentate gyrus.

As used herein, a “preferred local administration” does not mean that all the viral particles are administered to said areas of the brain, but a majority, for example at least 50%, at least 60%, at least 70%, or at least 80% (vg) of the viral particles are administered to said areas.

With administration in the cerebrospinal space, neuronal transduction is dependent of cerebrospinal fluid circulation dynamics, therefore expected to be observed (1) in periventricular areas, i.e. areas in close proximity to the cerebral ventricles, (2) through a non-specific manner, i.e. neurons will be transduced by diffusion either from the ventricles or from the subarachnoid space, with strong labeling expected to be observed in upper cortical layers I-IV (e.g. by diffusion from the subaracnoid space) and (3) in brain areas such as the cerebellum and the hippocampus that are not connected to the putamen. Transduction from the ventricular system of neurons of deep brain areas such as the substantia nigra would be very unlikely bearing in mind that the substantia nigra is located far away from the ventricles, therefore very difficult to be transfected by passive diffusion.

In contrast to the administration in the cerebrospinal space, the administration of a viral vector in the caudate putamen nuclei presents several advantages such as a specific transduction of neurons located in cerebral cortex, thalamus, amygdala, substantia nigra pars compacta, and dorsal raphe nuclei innervating the injection site and circuit-specific retrograde spread in brain areas known to innervate the putamen for instance in layer V of the cortical areas projecting to the putamen without retrograde spread to unexpected areas (e.g. lack of retrograde transport to areas known to not to innervate the putamen).

Thus, the intraparenchymal route may facilitate local administration of a viral particle to the caudate-putamen nuclei, thus facilitating retrograde dissemination of a transgene to any brain area innervating the injection site.

In a preferred embodiment, a viral particle can be administered to the human subject or patient via intraparenchymal route to the caudate-putamen nuclei, in a volume comprised within a range of 50 to 1000 μL, preferably 200 to 700 μL, per putamen, at a concentration preferably comprised within the range of 10¹³-10¹⁴ vg/mL (vg: viral genomes). In a particular embodiment, said viral particle is administered at an injection debit within the range of 0.5 to 5 μL/min preferably during 2 to 6 hours. Such a high injection rate of the viral particle increases virus stability and allows a better management of patients.

In certain embodiments, said viral particle is selected among rAAV particles, preferably including capsid proteins selected from the group consisting of: AAV2, AAV5, AAV9, AAV-MNM004, AAV-MNM008, and AAV TT serotypes.

In certain embodiments, said viral particle is an AAVretro which includes capsid proteins selected among the following variant serotypes: AAV2-retro, AAV-MNM004, AAV-MNM008 and AAV-TT.

In one embodiment, AAV-TT particle can be administered to the human subject or patient via intraparenchymal route to the caudate-putamen nuclei, in a volume comprised within a range of 50 to 1000 μL, preferably 200 to 700 μL per putamen, at a concentration preferably comprised within the range of 10¹³-10¹⁴ vg/mL (vg: viral genomes). In a particular embodiment, said viral particle is administered at an injection debit within the range of 0.5 to 5 μL/min preferably during 2 to 6 hours.

In another embodiment, an AAV-9 particle can be administered to the human subject or patient via intraparenchymal route to the caudate-putamen nuclei, in a volume comprised within a range of 50 to 1000 μL, preferably 200 to 700 μL per putamen, at a concentration preferably comprised within the range of 10¹³-10¹⁴ vg/mL (vg: viral genomes). In a particular embodiment, said viral particle is administered at an injection debit within the range of 0.5 to 5 μL/min preferably during 2 to 6 hours.

In one embodiment the intraparenchymal route may facilitate preferred local administration of an AAV to the caudate-putamen nuclei, thus facilitating retrograde dissemination of GBA1 transgene to any brain area innervating the injection site.

The present disclosure relates to a viral particle, preferably AAV particles comprising GBA1 transgene according to the present disclosure for use in the treatment of a neurodegenerative disease such as tauopathies wherein said viral particle is administered via intraparenchymal route to the caudate-putamen nuclei.

In a preferred embodiment, an AAV viral particle according to the disclosure can be administered to the human subject or patient for the treatment of tauopathies, such as Alzheimer's Disease via intraparenchymal route to the caudate-putamen nuclei, in a volume comprised within a range of 50 to 1000 μL, preferably 200 to 700 μL per putamen, at a concentration preferably comprised within the range of 10¹³-10¹⁴ vg/mL (vg: viral genomes). In a particular embodiment, said viral particle is administered at an injection debit within the range of 0.5 to 5 μL/min preferably during 2 to 6 hours.

In a particular embodiment, an AAV-TT according to the present disclosure can be administered to the human subject or patient for the treatment of tauopathies, such as Alzheimer's Disease via intraparenchymal route to the caudate-putamen nuclei.

Said AAV-TT particle according to the disclosure can be administered to the human subject or patient for the treatment of tauopathies, such as Alzheimer's Disease via intraparenchymal route to the caudate-putamen nuclei, in a volume comprised within a range of 50 to 1000 μL, preferably 200 to 700 μL per putamen, at a concentration preferably comprised within the range of 10¹³-10¹⁴ vg/mL (vg: viral genomes). In a particular embodiment, said viral particle is administered at an injection debit within the range of 0.5 to 5 μL/min preferably during 2 to 6 hours.

In a preferred embodiment, there is provided an recombinant Adeno-Associated Virus (rAAV) particle comprising a nucleic acid construct comprising a transgene encoding human glucocerebrosidase comprising a sequence selected from the group consisting of SEQ ID NO: 5, 6, 8, 17 and 18, typically a sequence selected from the group consisting of SEQ ID NO:1, 7 11, 12 and 19, wherein said nucleic acid construct further comprises a promoter operably-linked to said transgene, wherein said rAAV particle include AAV-TT capsid proteins comprising amino acid sequence of SEQ ID NO: 14 or sequence having at least 98.5%, preferably 99% or 99.5% identity with SEQ ID NO: 14, for use in the treatment of a neurodegenerative disease such as synucleopathies, preferably Gaucher disease (such as neuropathic Gaucher disease) or PD (such as sporadic PD), wherein said rAAV particle is administered via intraparenchymal route to the caudate-putamen nuclei, preferably in a volume comprised within a range of 50 to 1000 μL, preferably 200 to 700 μL per putamen, at a concentration preferably comprised within the range of 10¹³-10¹⁴ vg/mL (vg: viral genomes). In a particular embodiment, said viral particle is administered at an injection debit comprised within the range of 0.5 to 5 μL/min preferably during 2 to 6 hours.

In another embodiment, an AAV-9 according to the present disclosure can be administered to the human subject or patient for the treatment of tauopathies, such as Alzheimer's Disease, via intraparenchymal route to the caudate-putamen nuclei.

Said AAV-9 particle according to the disclosure can be administered to the human subject or patient for the treatment of tauopathies, such as Alzheimer's Disease via intraparenchymal route to the caudate-putamen nuclei, in a volume comprised within a range of 50 to 1000 μL, preferably 200 to 700 μL per putamen, at a concentration preferably comprised within the range of 10¹³-10¹⁴ vg/mL (vg: viral genome). In a particular embodiment, said viral particle is administered at an injection debit within the range of 0.5 to 5 μL/min preferably during 2 to 6 hours.

In another embodiment the intraparenchymal route may facilitate preferred local administration of an AAVretro to the dentate gyrus of the hippocampal formation, thus facilitating retrograde dissemination of GBA1 transgene to any brain area innervating the injection site, preferably neurons located in layers II and III of the entorhinal cortex.

The therapeutically effective amount of the product of the disclosure, or pharmaceutical composition that comprises it may vary according to factors such as the disease state, age, sex, and weight of the individual, and the ability of the product or pharmaceutical composition to elicit a desired response in the individual. Dosage regimens may be adjusted to provide the optimum therapeutic response.

A therapeutically effective amount is also typically one in which any toxic or detrimental effect of the product or pharmaceutical composition is outweighed by the therapeutically beneficial effects.

For any particular subject, specific dosage regimens may be adjusted over time according to the individual needs and the professional judgment of the person administering or supervising the administration of the compositions. Dosage ranges set forth herein are exemplary only and do not limit the dosage ranges that may be selected by medical practitioners.

In one embodiment, an AAV viral particle according to the disclosure can be administered to the human subject or patient for the treatment of tauopathies, such as Alzheimer's Disease, in an amount or dose comprised within a range of 10⁸-10¹⁴ vg/kg (vg: viral genomes; kg: subject's or patient's body weight).

### Kit

In another aspect, the disclosure further relates to a kit comprising a nucleic acid construct, viral vector, a host cell, viral particle or pharmaceutical composition as described above in one or more containers. The kit may include instructions or packaging materials that describe how to administer the nucleic acid construct, viral vector, viral particle, host cell or pharmaceutical composition contained within the kit to a patient. Containers of the kit can be of any suitable material, e.g., glass, plastic, metal, etc., and of any suitable size, shape, or configuration. In certain embodiments, the kits may include one or more ampoules or syringes that contain the products of the invention in a suitable liquid or solution form.

The following examples are provided by way of illustration, and they are not intended to be limiting of the present disclosure.

### EXAMPLES

In Vivo Dissemination Assay for Testing and Comparing AAV with Retrograde Transport

A test rAAV-GFP was prepared using standard methods for producing rAAV. The test rAAV-GFP used a nucleic acid construct with a GFP encoding sequence as the transgene, under the control of CAG promoter, and with ITRs of AAV2, said nucleic acid construct being packaged with capsid proteins of the AAV serotype to be tested for its retrograde transport property.

The in vivo dissemination assay included a first step of injecting said test rAAV-GFP by intraparenchymal injection of said rAAV-GFP into the post-commissural putamen of a non-human primate.

Then, the assay included a step of counting the number of GFP-expressing neurons in the cerebral cortex, substantia nigra, amygdala and caudal intralaminar nuclei one month post injection. Cell counting were carried out by taking advantage of Aiforia™, a whole-slide digital imaging and deep convolutional neuronal networks (CNN) algorithm designed for the automatic unbiased counting of immunoperoxidase-stained cells in brain tissue specimens (Penttinen et al., European Journal of Neuroscience 2018; 48:2354-2361).

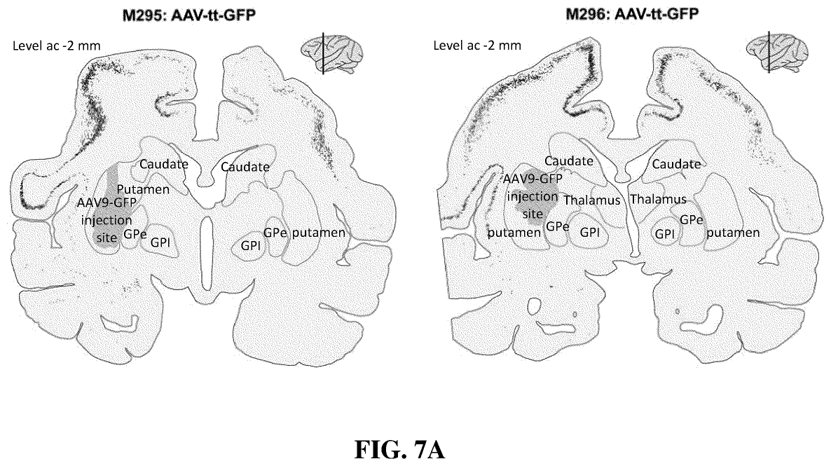

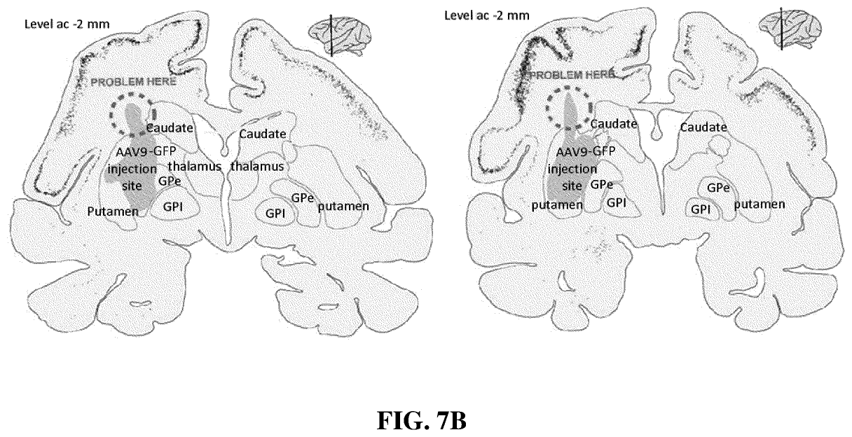

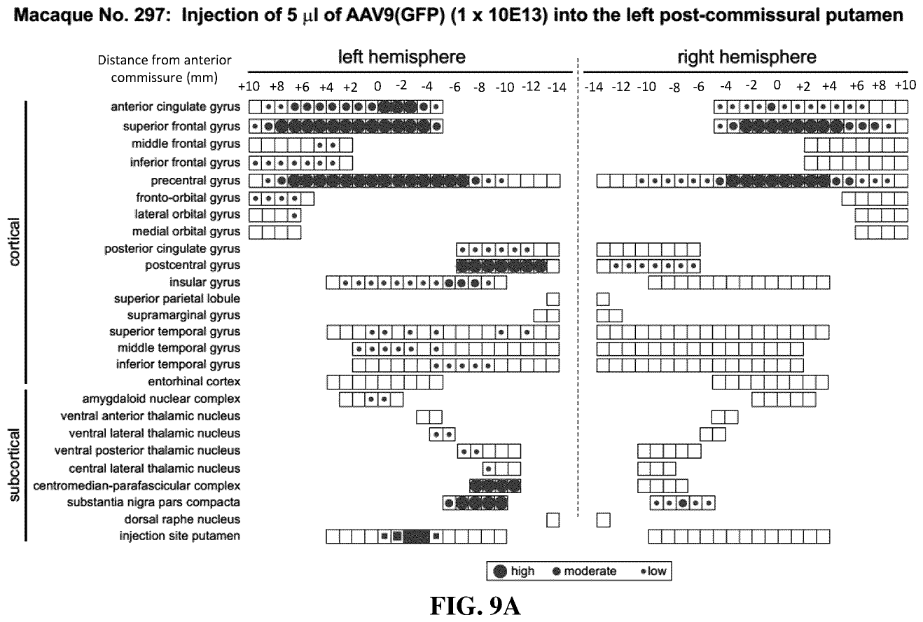

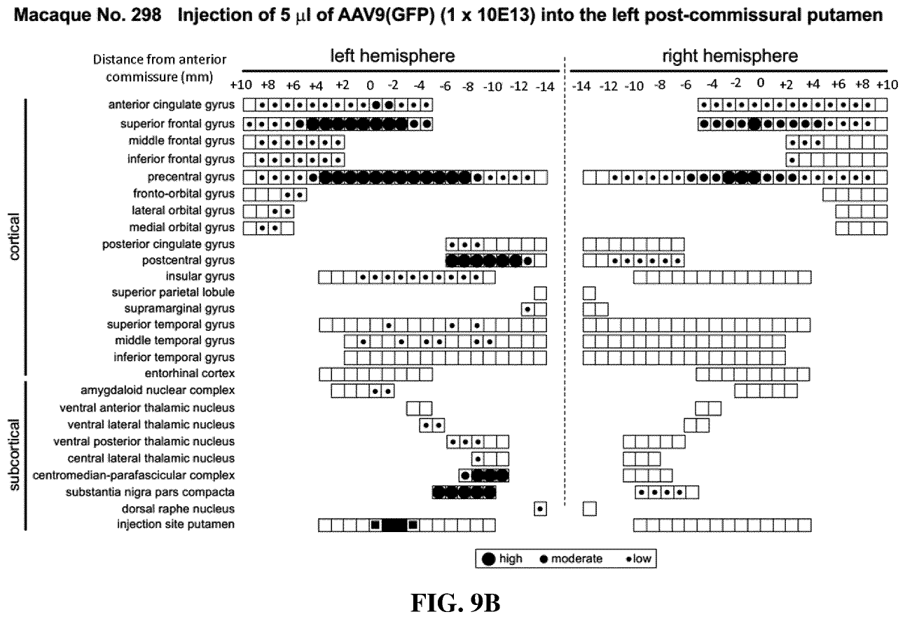

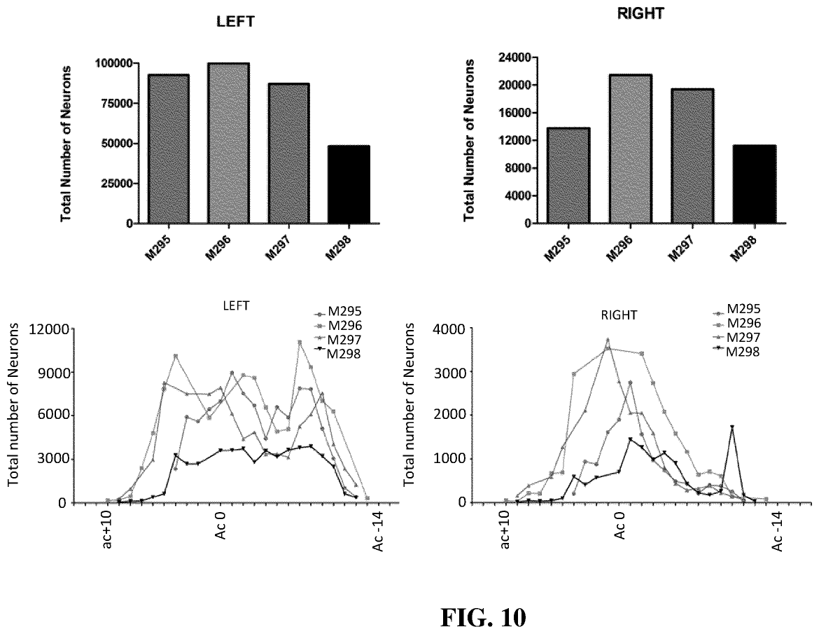

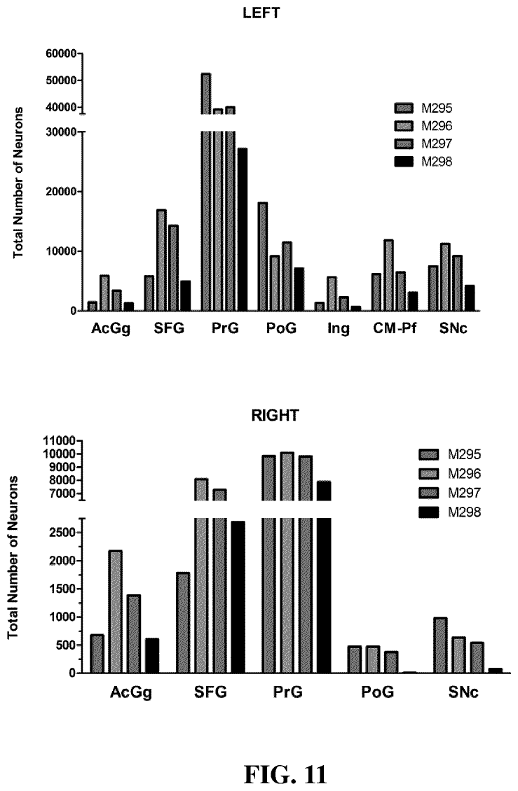

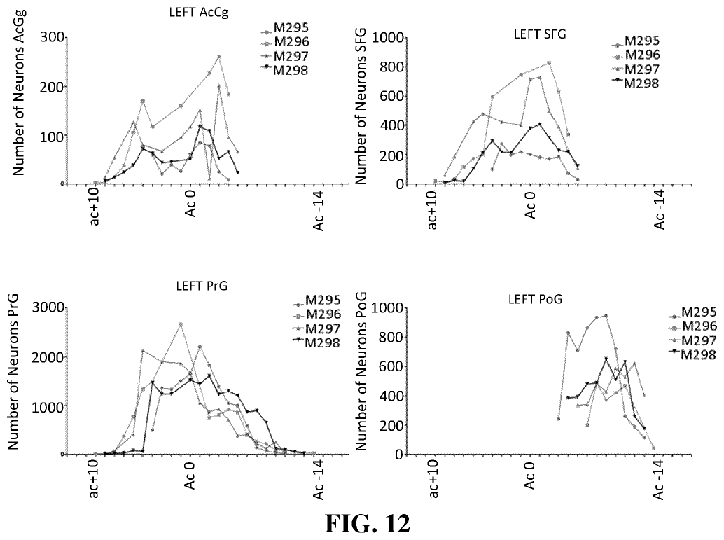

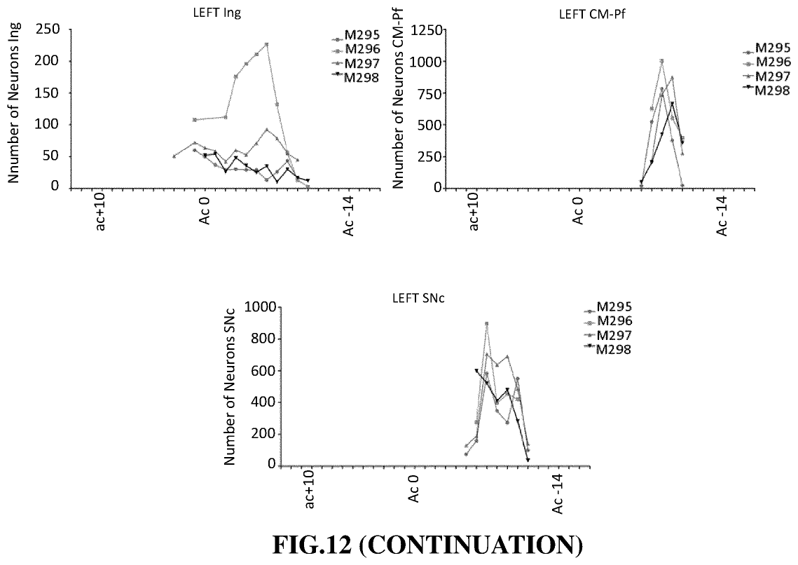

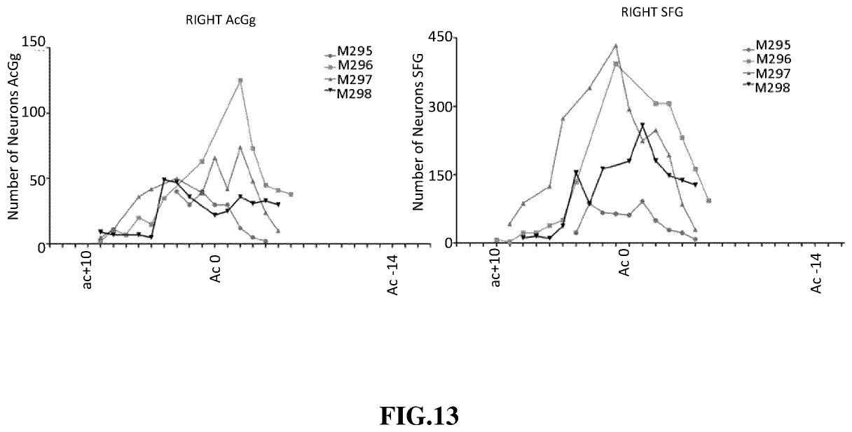

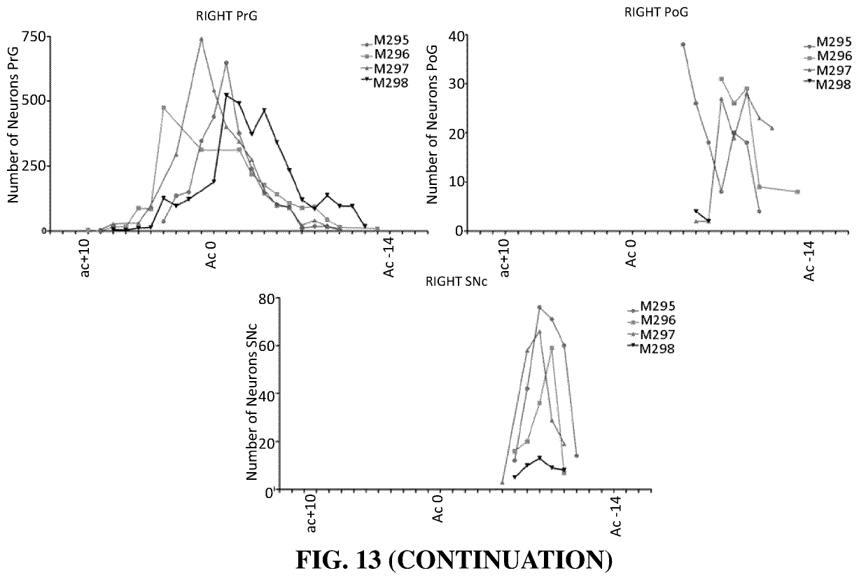

Neurons expressing GFP were visualized by immunoperoxidase stains, using anti-GFP antibodies. GFP-expressing neurons were automatically counted throughout the brain of the injected non-human primates. A preferential location of GFP-positive neurons occurred in deep layers of the cerebral cortex as illustrated in FIGS. 7A & 7B. Besides cortical areas, GFP-expressing neurons were quantified in all brain areas innervating the injected post-commissural putamen, particularly the substantia nigra pars compacta, the amygdala and the caudal intralaminar nuclei (FIGS. 7 to 13).

### 1. Studies in Mice:

The inventors have designed the following gene therapy product candidate:

Recombinant adeno-associated viral vector serotype 2/9 coding for the GBA1 gene under the control of a constitutive promoter GusB (rAAV2/9-GBA1), for example using the construct pAAV.nGUSB.GBA1 of SEQ ID NO:4 as described below.

The choice of a constitutive promoter (GusB) driving transgene expression is preferred here, bearing in mind the need for enhancing glucocerebrosidase activity both in neurons as well as in glial cells.

The viral vector may advantageously be delivered intraparenchymally, i.e. viral vector administration may be achieved through a direct injection into the desired brain area by means of stereotaxic surgery.

More specifically, the inventors hypothesized that glucocerebrosidase expression might be engaged in the processes leading to tau aggregation.

As a proof-of-concept for such working hypothesis, the inventors have conducted the experiments where wild-type mice (n=2) were bilaterally injected in the striatum with a recombinant adeno-associated viral vector (AAV) serotype 9 coding for the mutated form of human tau protein (Tau301L) under the control of a neuron specific synapsin promoter (rAAV2/9-Tau301L). Each striata received 1.0 microliters of 1.22×10E13 of the viral suspension.

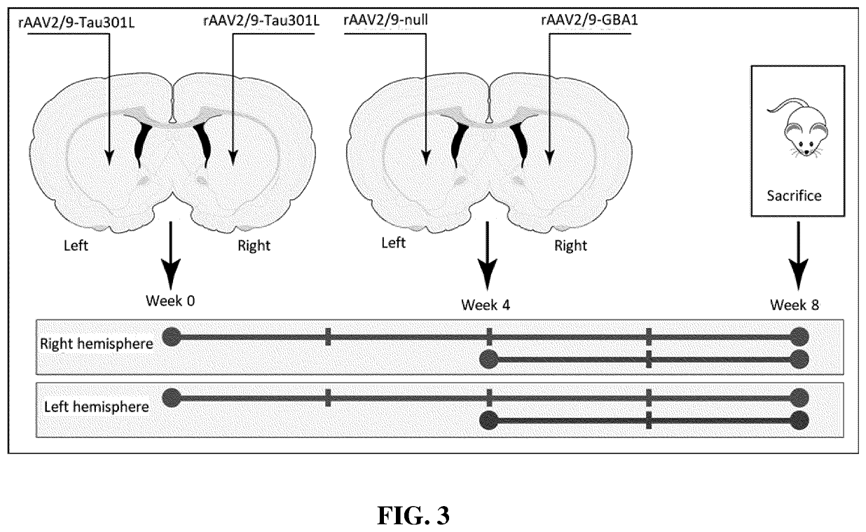

Next, once the tau-driven neurodegenerative processes were already ongoing but before reaching a non-returning point (e.g. 4 weeks post-delivery of rAAV2/9-Tau301L), a recombinant AAV2/9 coding for the GBA1 gene under the control of a constitutive promoter GusB (rAAV2/9-GBA1) was delivered into the right striatum (1.0 microliters of 1.25×10E13 of the viral suspension), together with an empty, control rAAV2/9 coding for no transgene (r-AAV2/9-null) injected into the left striatum. 3 weeks later (e.g. 8 weeks after initial delivery of rAAV2/9-Tau301L), animals were sacrificed and processed for neuropathological analysis. The experimental plan is summarized in FIG. 3.

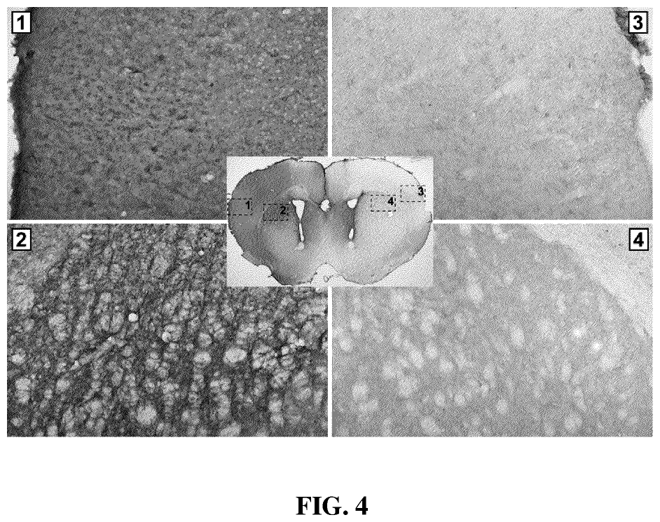

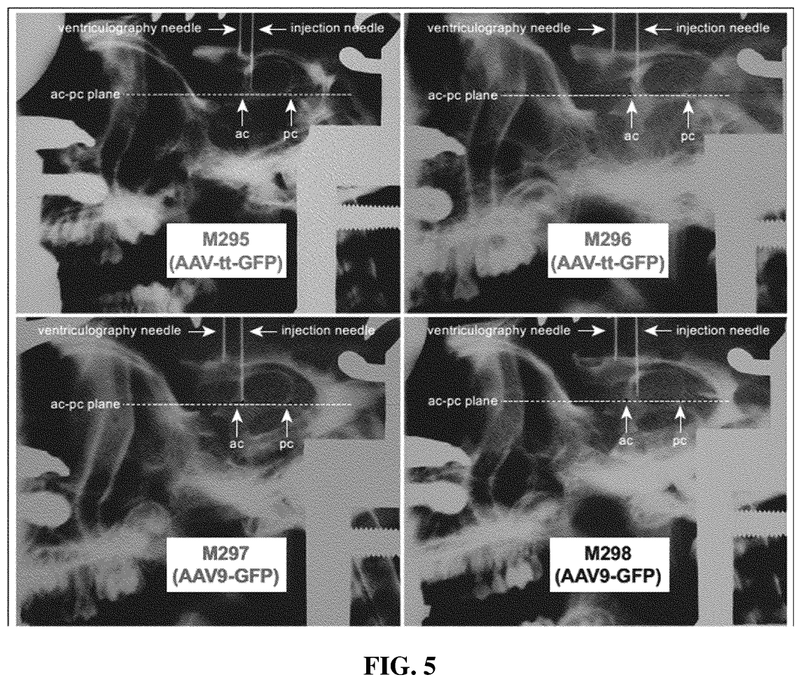

The conducted preliminary neuropathological analysis revealed that the rAAV2/9-GBA1 mediated enhancement of glucocerebrosidase surprisingly resulted in an almost complete clearance of tau aggregates throughout brain areas showing tau-related neuropathology, i.e. the striatum and the cerebral cortex (FIG. 4).

### 2. Biodistribution and Comparative Performance of AAV-TT-GFP and AAV9-GFP in the Nonhuman Primate Brain

### 2.1 Conducted Experiments

Up to four adult juvenile male Macaca fascicularis primates (body weight between 3.0 to 3.4 Kg) were injected with either 5 μL of AAV-TT-GFP (1×10¹³ vg/mL; 2 animals) or with 5 μl of AAV9-GFP (1×10¹³ vg/mL; 2 animals). Both AAVs were coding for GFP under the control of a CAG promoter.

AAVs were administered through ventriculography-assisted stereotaxic surgery by taking advantage of a Hamilton syringe. Pressure-injections were achieved in pulses of 0.5 μL/min. In non-human primate the debit is adjusted to the lower range. However, in human trials high injection speed allows virus stability, and better patient management and debit can range from 0.5 μL to 5 μL/min. Once AAV delivery was completed, the injection needle was left in place for additional 10 min to minimize AAV reflux through the injection tract (FIG. 5). Just before surgery, body fluid samples (blood and CSF) were collected and stored at −80° C.

Animals were sacrificed one month post-AAV delivery through intracardiac perfusion. Before sacrifice, body fluid samples (blood and CSF) were collected and stored at −80° C. The perfusates consisted of a saline Ringer solution followed by a buffered solution of paraformaldehyde (3,000 ml/animal) and by 1,000 mL of a cryoprotective solution made of 10% glycerin and 1% DMSO in phosphate buffer 0.1 M, pH 7.3.

During perfusion with the Ringer solution, fresh tissue samples (e.g. unfixed) were taken from a number of peripheral organs, these including heart, lung, liver, spleen, pancreas, kidney, testis and striatal muscle. Samples were frozen on dry ice and stored at −80° C.

Once perfusion is completed, the brain was removed from the skull and brain blocks of approximately 1 cm wide were made and stored in a cryoprotective solution made of 20% glycerin and 2% DMSO in phosphate buffer 0.1 M, pH 7.3 (pia matter removed from all brain blocks). Samples from fixed peripheral organs were obtained (heart, lung, liver, spleen, pancreas, kidney, testis, retroperitoneal ganglia, pineal gland and striatal muscle) and further embedded in paraffin.

After a minimum of 48 h in the cryoprotective solution, 10 series of frozen coronal brain sections (40 μm-thick) were made in a sliding microtome and collected in the cryoprotective solution. One entire series of sections (e.g. comprising every 10th section of the monkey brain) was processed for the immunoperoxidase detection of GFP by taking advantage of a primary polyclonal antibody raised in rabbit. Upon incubation with a biotinylated goat anti-rabbit IgG, sections were then incubated with and ABC kit and finally stained using H₂O₂-DAB solution. Once stained is completed, free-floating sections were mounted on microscopy slides, air-dried overnight and coverslipped with entellan. Stained sections were scanned using an Aperio CS2 slide scanner (Leica) and processed using dedicated software.

### 2.2 Results

Labeling with either AAV-TT-GFP or with AAV9-GFP was only found throughout brain territories known to innervate the post-commissural putamen, whereas not even a single labeled neuron was observed in brain territories not innervating the injection site (e.g. the hippocampus, cerebellum, etc). Moreover, obtained retrograde labeling was of “Golgi-like” morphology, i.e. neuronal labeling was not limited to cell somata and indeed extends over distal dendrites, particularly in locations throughout the cerebral cortex. It is also worth noting that small dendritic processes such as dendritic spines are sometimes even visible.

### Events at the Injection Site

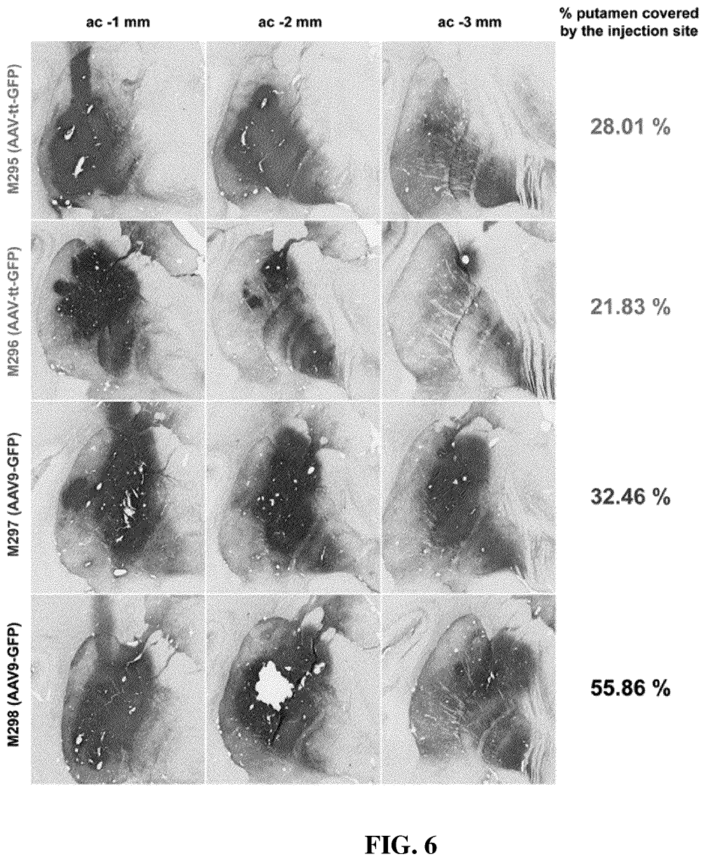

Both injections of AAV-TT-GFP were accurate and properly located within the boundaries of the post-commissural putamen. Obtained sizes for the injection sites were consistently smaller for AAV-TT-GFP than for AAV9-GFP, covering 28.01% and 21.83% in animals M295 and M296 (injected with AAV-TT-GFP), whereas in animals injected with AAV9-GFP (M297 and M298), 32.46% and 55.86% of the post-commissural putamen was comprised within the injection sites, respectively (FIG. 6). Both injections with AAV-TT-GFP showed a completely lack of AAV uptake through the injection tract (e.g. these are both very clean injections). By contrast, injections performed with AAV9-GFP exhibited a moderate-to-high uptake through the injection tract, meaning that it is very likely that obtained results are contaminated by false positive labeling (probably particularly notorious within cortical territories) due to AAV9-GFP uptake by white matter tracts located above the post-commissural putamen. Troubles related to false positive results are illustrated in FIG. 7B. Furthermore, the delivery of AAV9-GFP in animal M297 has spread beyond the boundaries of the post-commissural putamen, and also includes a substantial part of the external globus pallidus (GPe).

### The Potency of Retrograde Spread

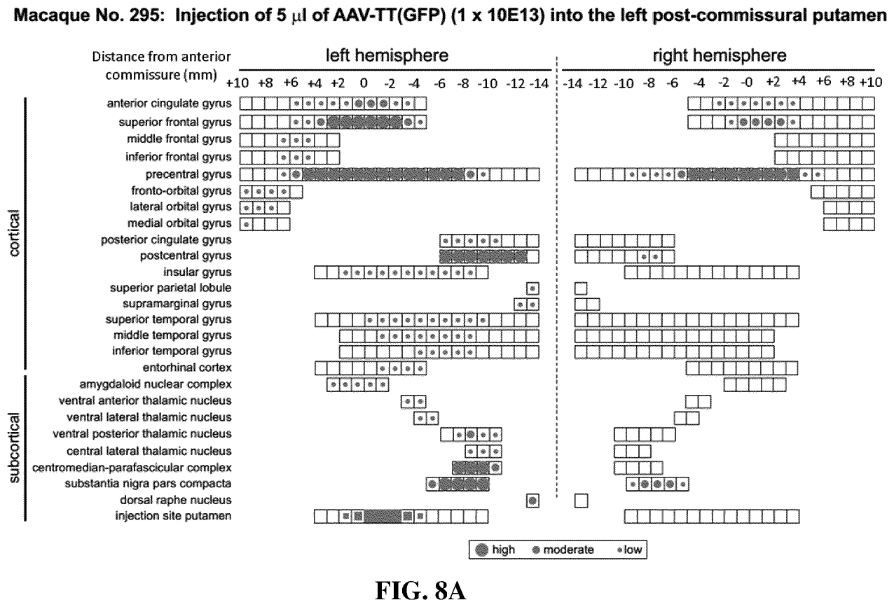

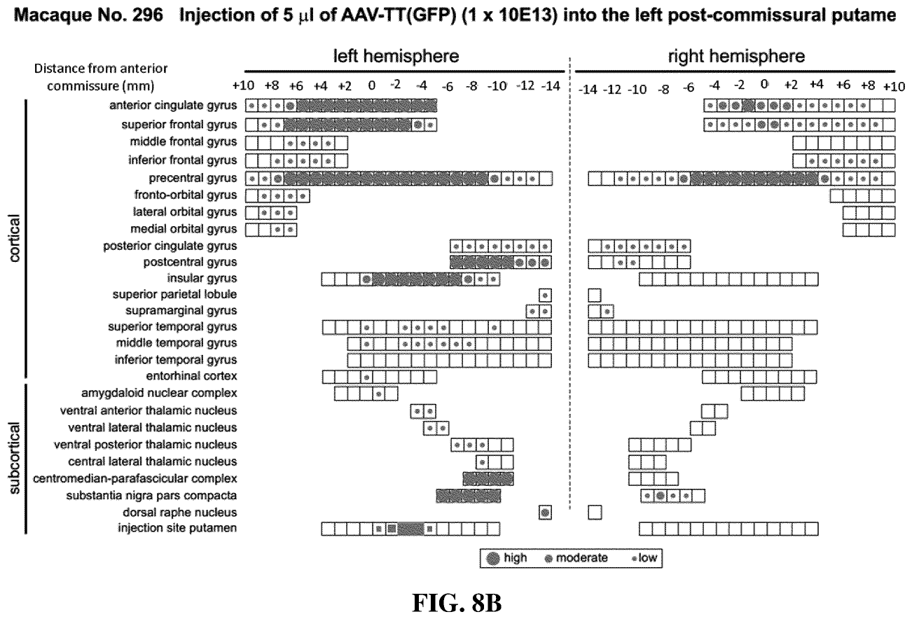

Both the total numbers and observed intensities of retrogradely-labeled neurons are directly related to the extent of the injection sites. In other words, higher numbers of GFP+ neurons are expected from injection sites covering larger territories of the post-commissural putamen. In this regard, and besides providing an accurate quantification of the number of neurons observed in each region of interest, this final number needs to be corrected by the extent of the post-commissural putamen area being covered by the injection site. Numbers of neurons are provided based on the quantification done with Aiforia®. In an attempt to properly compare the performance of AAV-TT vs. AAV9, obtained raw data need to be standardized by taking into consideration the extent of the injection site. Accordingly, a correction factor based on the size of the injection site was calculated to properly estimate the expected retrograde spread of each AAV. Correction factors were ×3.57 for M295, ×4.58 for M296, ×3.08 for M297 and ×1.79 for M298. Correction factors were used for generating data showed in FIGS. 8-13.

### Brain Areas Showing Strongest Labeling

In all animals (AAV-TT-GFP and AAV9-GFP), the strongest labeling was observed in the superior frontal gyms and in the precentral gyrus (FIG. 8). Other cortical areas consistently showing GFP+ neurons (although to a lower extent) are the anterior cingulate cortex, the postcentral gyms and the insular gyms. Sparse neuronal labeling was observed in the middle frontal gyrus, inferior frontal gyrus, orbital frontal cortex (frontal orbital, lateral orbital and medial orbital territories), superior, middle and inferior temporal gyri, as well as in the superior parietal lobule and the supramarginal gyms. Furthermore, GFP+ neurons were consistently found in the contralateral cortex as mirror-like representations of the ipsilateral cortex (obviously containing a much lower number of GFP+ neurons). Results are fully consistent with what was expected and indeed very relevant, bearing in mind that upon AAV delivery in the post-commissural putamen (the motor-related putamenal territories), the strongest labeling was observed in both the precentral and superior frontal gyri (cortical gyri containing the primary motor cortex and the supplementary motor area, respectively). Regarding subcortical labeling, two structures are particularly relevant, namely the substantia nigra pars compacta (SNc) and the centromedian-parafascicularis complex (CM-Pf). Moreover, the amount of GFP+ neurons observed in the CM-Pf is very impressive, although expected, bearing in mind that the CM-Pf thalamic complex is the main source of thalamostriatal projections. Besides CM-Pf, sparse labeling was also found in the ventral anterior, ventral lateral and ventral posteromedial thalamic nuclei, centrolateral and paracentral intralaminar nuclei and the dorsal raphe nucleus (a small brainstem nucleus known to be the main source of serotoninergic projections to the putamen). Furthermore, observed labeling at the level of the amygdaloid complex is lower than initially expected for both AAV types. Although the amygdaloid complex has often been viewed as another source of afferents to the putamen (together with the cortex, thalamus and substantia nigra), data obtained with AAV-TT and with AAV9 clearly suggested that the importance of this anatomical pathway has been likely overestimated in earlier anatomical studies.

### Striatal Afferent Systems

Although the present study was not designed for this purpose, the conducted quantification allows to numerically estimate the “weight” of each different striatal afferent system, namely the corticostriatal pathways (ipsi- and contralateral), thalamostriatal and nigrostriatal projections. Obtained data showed that ipsilateral corticostriatal projections are by far the most abundant ones (69.37% of total striatal afferents on average), followed by contralateral corticostriatal-projecting neurons (15.99% of total striatal afferents), then nigrostriatal projections (7.99% on average) and finally the thalamostriatal projections arising from the centromedian-parafascicular thalamic complex (6.67%). In this regard, it is also worth noting that although the contralateral corticostriatal pathway has often been neglected in most studies dealing with basal ganglia function and dysfunction, this projection roughly represents up to 16% of total striatal afferents, a percentage clearly above the ones related to thalamostriatal and nigrostriatal projections.

Obtained results supported a superior performance of AAV-TT-GFP when compared to AAV9-GFP. A deep comparison of the results showed that AAV-TT is a better candidate than AAV9. AAV-TT holds some important advantages, particularly when dealing with a higher “potency” in terms of retrograde spread, together with the lack of uptake through the injection tract. The use of AAV-TT presents a complete lack of false positives.

### Sequences for Use in Practicing the Invention

Sequences for use in practicing the invention are described below (non-limiting list):

SEQ ID NO: 1: Human GBA1 coding nucleotide sequence
(optimized sequence as used in the Examples):
ATGGCTGGCAGTCTTACAGGTCTCCTGCTCCTGCAAGCTGTCTCTTGGGCTTCTGGGGCCAGGCCCTG
TATCCCCAAATCCTTTGGATACTCATCTGTGGTGTGIGTTTGTAATGCCACTTATTGTGATAGCTTTG
ACCCCCCCACCTTTCCTGCACTGGGCACCTTTTCAAGGTATGAATCTACCAGGTCTGGGAGGAGGATG
GAGCTGAGTATGGGGCCCATCCAAGCAAACCATACTGGCACTGGCTTGCTGCTGACACTGCAACCTGA
ACAGAAGTTCCAGAAAGTGAAGGGCTTTGGAGGAGCCATGACTGATGCTGCTGCCCTCAATATTTTGG
CCCTGAGCCCCCCTGCTCAGAATCTCCTTTTGAAATCATACTTCTCTGAGGAGGGAATTGGATACAAT
ATCATCAGGGTGCCAATGGCCTCATGTGACTTTAGTATTAGGACTTACACCTATGCTGATACCCCTGA
TGATTTCCAGCTGCATAACTTCTCATTGCCTGAGGAGGATACCAAATTGAAGATCCCACTCATTCACA
GGGCCCTGCAACTGGCTCAGAGACCAGTGTCATTGCTGGCCTCCCCCTGGACCTCCCCAACTTGGCTC
AAAACCAATGGGGCTGTCAATGGTAAGGGCTCTCTTAAGGGGCAGCCTGGAGACATTTACCATCAGAC
CTGGGCCAGGTATTTIGTGAAGTTCCTGGATGCTTATGCTGAGCACAAATTGCAATTTTGGGCTGTTA
CAGCTGAGAATGAACCCTCTGCAGGACTGCTGTCTGGCTATCCTTTCCAGTGCCTGGGCTTTACCCCT
GAGCATCAGAGGGATTTCATTGCCAGGGACCTGGGACCTACTCTTGCCAATAGCACACACCATAATGT
GAGGCTTCTGATGCTTGATGACCAGAGACTTCTGCTGCCACACTGGGCCAAGGTTGTCCTGACAGATC
CTGAGGCTGCCAAGTATGTTCATGGGATTGCTGTGCACTGGTATCTGGACTTCCTTGCTCCAGCTAAG
GCCACCCTGGGAGAAACACACAGGTTGTTTCCCAATACAATGCTTTTTGCATCAGAGGCCTGTGTGGG
CAGTAAATTTTGGGAGCAGTCTGTTAGGCTGGGGAGCTGGGATAGAGGAATGCAATACTCCCATTCTA
TCATCACCAATCTGCTCTACCATGTGGTGGGGTGGACTGACTGGAACCTTGCCCTTAACCCTGAGGGT
GGCCCCAATTGGGTCAGGAATTTTGTGGATAGTCCCATCATTGTGGATATCACCAAGGACACATTCTA
TAAGCAACCAATGTTCTATCACCIGGGTCACTTTAGTAAGTTTATCCCTGAGGGGTCCCAGAGGGTGG
GACTGGTGGCTTCCCAGAAGAATGATCTGGATGCTGTGGCCCTGATGCACCCTGATGGCAGTGCTGTG
GTTGTTGTTCTCAATAGAAGCTCTAAAGATGTGCCCTTGACCATCAAAGATCCAGCTGTGGGATTTCT
GGAAACAATTTCCCCTGGTTATAGCATCCACACTTACCTTTGGAGAAGGCAGTGA
SEQ ID NO: 2: nGUSB promoter nucleotide sequence
ATTCCTGCTGGGAAAAGCAAGTGGAGGTGCTCCTTGAAGAAACAGGGGGATCCCACCGATCTCAGGGG
TTCTGTTCTGGCCTGCGGCCCTGGATCGTCCAGCCTGGGTCGGGGTGGGGAGCAGACCTCGCCCTTAT
CGGCTGGGGCTGAGGGTGAGGGTCCCGTTTCCCCAAAGGCCTAGCCTGGGGTTCCAGCCACAAGCCCT
ACCGGGCAGCGCCCGGCCCCGCCCCTCCAGGCCTGGCACTCGTCCTCAACCAAGATGGCGCGGATGGC
TTCAGGCGCATCACGACACCGGCGCGTCACGCGACCCGCCCTACGGGCACCTCCCGCGCTTTTCTTAG
CGCCGCAGACGGTGGCCGAGCGGGGGACCGGGAAGCATGGCCCGGGCT
SEQ ID NO: 3: Bovine growth hormone (BGH) polyadenylation signal
GCCCTGTGCCTTCTAGTTGCCAGCCATCTGTTGTTTGCCCCTCCCCCGTGCCTTCCTTGACCCTGGAA
GGTGCCACTCCCACTGTCCTTTCCTAATAAAATGAGGAAATTGCATCGCATTGTCTGAGTAGGIGTCA
TTCTATTCTGGGGGGTGGGGTGGGGCAGGACAGCAAGGGGGAGGATTGGGAAGACAATAGCAGGCATG
CACT
SEQ ID NO: 4: pAAV.nGUSB.GBA1 nucleotide sequence
TCGCGCGTTTCGGTGATGACGGTGAAAACCTCTGACACATGCAGCTCCCGGAGACGGTCACAGCTTGT
CTGTAAGCGGATGCCGGGAGCAGACAAGCCCGTCAGGGCGCGTCAGCGGGTGTTGGCGGGTGTCGGGG
CTGGCTTAACTATGCGGCATCAGAGCAGATTGTACTGAGAGTGCACCATATGCGGTGTGAAATACCGC
ACAGATGCGTAAGGAGAAAATACCGCATCAGGCGCCATTCGCCATTCAGGCTGCGCAACTGTTGGGAA
GGGCGATCGGTGCGGGCCTCTTCGCTATTACGCCAGCTGGCGAAAGGGGGATGTGCTGCAAGGCGATT
AAGTTGGGTAACGCCAGGGTTTTCCCAGTCACGACGTTGTAAAACGACGGCCAGTGAATTCGAGCTCG
GTACCTCGCGAATGCATCTAGAGGCCACTCCCTCTCTGCGCGCTCGCTCGCTCACTGAGGCCGGGCGA
CCAAAGGTCGCCCGACGCCCGGGCTTTGCCCGGGCGGCCTCAGTGAGCGAGCGAGCGCGCAGAGAGGG
AGTGGCCAACTCCATCACTAGGGGTTCCTGGAGGGGTGGAGTCGTGACAGATCTGAATTCCTGCTGGG
AAAAGCAAGTGGAGGTGCTCCTTGAAGAAACAGGGGGATCCCACCGATCTCAGGGGTTCTGTTCTGGC
CTGCGGCCCTGGATCGTCCAGCCTGGGTCGGGGTGGGGAGCAGACCTCGCCCTTATCGGCTGGGGCTG
AGGGTGAGGGTCCCGTTTCCCCAAAGGCCTAGCCTGGGGTTCCAGCCACAAGCCCTACCGGGCAGCGC
CCGGCCCCGCCCCTCCAGGCCTGGCACTCGTCCTCAACCAAGATGGCGCGGATGGCTTCAGGCGCATC
ACGACACCGGCGCGTCACGCGACCCGCCCTACGGGCACCTCCCGCGCTTTTCTTAGCGCCGCAGACGG
TGGCCGAGCGGGGGACCGGGAAGCATGGCCCGGGCTGCAGCTCTAAGGTAAATATAAAATTTTTAAGT
GTATAATGTGTTAAACTACTGATTCTAATTGTTTCTCTCTTTTAGATTCCAACCTTTGGAACTCAATT
CAGCCACCATGGCTGGCAGTCTTACAGGTCTCCTGCTCCTGCAAGCTGTCTCTTGGGCTTCTGGGGCC
AGGCCCTGTATCCCCAAATCCTTTGGATACTCATCTGTGGTGTGTGTTTGTAATGCCACTTATTGTGA
TAGCTTTGACCCCCCCACCTTTCCTGCACTGGGCACCTTTTCAAGGTATGAATCTACCAGGTCTGGGA
GGAGGATGGAGCTGAGTATGGGGCCCATCCAAGCAAACCATACTGGCACTGGCTTGCTGCTGACACTG
CAACCTGAACAGAAGTTCCAGAAAGTGAAGGGCTTTGGAGGAGCCATGACTGATGCTGCTGCCCTCAA
TATTTTGGCCCTGAGCCCCCCTGCTCAGAATCTCCTTTTGAAATCATACTTCTCTGAGGAGGGAATTG
GATACAATATCATCAGGGIGCCAATGGCCTCATGTGACTTTAGTATTAGGACTTACACCTATGCTGAT
ACCCCTGATGATTICCAGCTGCATAACTTCTCATTGCCTGAGGAGGATACCAAATTGAAGATCCCACT
CATTCACAGGGCCCTGCAACTGGCTCAGAGACCAGTGTCATTGCTGGCCTCCCCCTGGACCTCCCCAA
CTTGGCTCAAAACCAATGGGGCTGTCAATGGTAAGGGCTCTCTTAAGGGGCAGCCTGGAGACATTTAC
CATCAGACCTGGGCCAGGTATTTIGTGAAGTTCCTGGATGCTTATGCTGAGCACAAATTGCAATTTTG
GGCTGTTACAGCTGAGAATGAACCCTCTGCAGGACTGCTGTCTGGCTATCCTTTCCAGTGCCTGGGCT
TTACCCCTGAGCATCAGAGGGATTTCATTGCCAGGGACCTGGGACCTACTCTTGCCAATAGCACACAC
CATAATGIGAGGCTTCTGATGCTTGATGACCAGAGACTTCTGCTGCCACACTGGGCCAAGGTTGTCCT
GACAGATCCTGAGGCTGCCAAGTATGTTCATGGGATTGCTGTGCACTGGTATCTGGACTTCCTTGCTC
CAGCTAAGGCCACCCTGGGAGAAACACACAGGTTGTTTCCCAATACAATGCTTTTTGCATCAGAGGCC
TGTGTGGGCAGTAAATTTTGGGAGCAGTCTGTTAGGCTGGGGAGCTGGGATAGAGGAATGCAATACTC
CCATTCTATCATCACCAATCTGCTCTACCATGTGGTGGGGTGGACTGACTGGAACCTTGCCCTTAACC
CTGAGGGTGGCCCCAATTGGGTCAGGAATTTTGTGGATAGTCCCATCATTGTGGATATCACCAAGGAC
ACATTCTATAAGCAACCAATGTTCTATCACCTGGGTCACTTTAGTAAGTTTATCCCTGAGGGGTCCCA
GAGGGTGGGACTGGTGGCTTCCCAGAAGAATGATCTGGATGCTGTGGCCCTGATGCACCCTGATGGCA
GTGCTGTGGTTGTTGTTCTCAATAGAAGCTCTAAAGATGTGCCCTTGACCATCAAAGATCCAGCTGTG
GGATTTCTGGAAACAATTTCCCCTGGTTATAGCATCCACACTTACCTTTGGAGAAGGCAGTGAAAATG
AAGGCCTGATAATTGCACCACCAGGCCTGATAGGCCCTGTGCCTTCTAGTTGCCAGCCATCTGTTGTT
TGCCCCTCCCCCGTGCCTTCCTTGACCCTGGAAGGTGCCACTCCCACTGTCCTTTCCTAATAAAATGA
GGAAATTGCATCGCATTGTCTGAGTAGGTGTCATTCTATTCTGGGGGGTGGGGTGGGGCAGGACAGCA
AGGGGGAGGATTGGGAAGACAATAGCAGGCATGCACTAGTCCACTCCCTCTCTGCGCGCTCGCTCGCT
CACTGAGGCCGGGCGACCAAAGGTCGCCCGACGCCCGGGCTTTGCCCGGGCGGCCTCAGTGAGCGAGC
GAGCGCGCAGAGAGGGATCTAGATATCGGATCCCGGGCCCGTCGACTGCAGAGGCCTGCATGCAAGCT
TGGCGTAATCATGGTCATAGCTGTTTCCTGTGTGAAATTGTTATCCGCTCACAATTCCACACAACATA
CGAGCCGGAAGCATAAAGTGTAAAGCCTGGGGTGCCTAATGAGTGAGCTAACTCACATTAATTGCGTT
GCGCTCACTGCCCGCTTTCCAGTCGGGAAACCTGTCGTGCCAGCTGCATTAATGAATCGGCCAACGCG
CGGGGAGAGGCGGTTTGCGTATTGGGCGCTCTTCCGCTTCCTCGCTCACTGACTCGCTGCGCTCGGTC
GTTCGGCTGCGGCGAGCGGTATCAGCTCACTCAAAGGCGGTAATACGGTTATCCACAGAATCAGGGGA
TAACGCAGGAAAGAACATGTGAGCAAAAGGCCAGCAAAAGGCCAGGAACCGTAAAAAGGCCGCGTTGC
TGGCGTTTTTCCATAGGCTCCGCCCCCCTGACGAGCATCACAAAAATCGACGCTCAAGTCAGAGGTGG
CGAAACCCGACAGGACTATAAAGATACCAGGCGTTTCCCCCTGGAAGCTCCCTCGTGCGCTCTCCTGT
TCCGACCCTGCCGCTTACCGGATACCTGTCCGCCTTTCTCCCTTCGGGAAGCGTGGCGCTTTCTCATA
GCTCACGCTGTAGGTATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAACCCGGTAAGACACGA
CTTATCGCCACTGGCAGCAGCCACTGGTAACAGGATTAGCAGAGCGAGGTATGTAGGCGGTGCTACAG
AGTTCTTGAAGTGGTGGCCTAACTACGGCTACACTAGAAGAACAGTATTTGGTATCTGCGCTCTGCTG
AAGCCAGTTACCTTCGGAAAAAGAGTTGGTAGCTCTTGATCCGGCAAACAAACCACCGCTGGTAGCGG
TGGTTTTTTTGTTTGCAAGCAGCAGATTACGCGCAGAAAAAAAGGATCTCAAGAAGATCCTTTGATCT
TTTCTACGGGGTCTGACGCTCAGTGGAACGAAAACTCACGTTAAGGGATTTTGGTCATGAGATTATCA
AAAAGGATCTTCACCTAGATCCTTTTAAATTAAAAATGAAGTTTTAAATCAATCTAAAGTATATATGA
GTAAACTTGGTCTGACAGTTACCAATGCTTAATCAGTGAGGCACCTATCTCAGCGATCTGTCTATTTC
GTTCATCCATAGTTGCCTGACTCCCCGTCGTGTAGATAACTACGATACGGGAGGGCTTACCATCTGGC
CCCAGTGCTGCAATGATACCGCGAGACCCACGCTCACCGGCTCCAGATTTATCAGCAATAAACCAGCC
AGCCGGAAGGGCCGAGCGCAGAAGTGGTCCTGCAACTTTATCCGCCTCCATCCAGTCTATTAATTGTT
GCCGGGAAGCTAGAGTAAGTAGTTCGCCAGTTAATAGTTTGCGCAACGTTGTTGCCATTGCTACAGGC
ATCGTGGTGTCACGCTCGTCGTTTGGTATGGCTTCATTCAGCTCCGGTTCCCAACGATCAAGGCGAGT
TACATGATCCCCCATGTTGTGCAAAAAAGCGGTTAGCTCCTTCGGTCCTCCGATCGTTGTCAGAAGTA
AGTTGGCCGCAGTGTTATCACTCATGGTTATGGCAGCACTGCATAATTCTCTTACTGTCATGCCATCC
GTAAGATGCTTTTCTGTGACTGGTGAGTACTCAACCAAGTCATTCTGAGAATAGTGTATGCGGCGACC
GAGTTGCTCTTGCCCGGCGTCAATACGGGATAATACCGCGCCACATAGCAGAACTTTAAAAGTGCTCA
TCATTGGAAAACGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTGGGTGAGCAAA
AACAGGAAGGCAAAATGCCGCAAAAAAGGGAATAAGGGCGACACGGAAATGTTGAATACTCATACTCT
TCCTTTTTCAATATTATTGAAGCATTTATCAGGGTTATTGTCTCATGAGCGGATACATATTTGAATGT
ATTTAGAAAAATAAACAAATAGGGGTTCCGCGCACATTTCCCCGAAAAGTGCCACCTGACGTCTAAGA
AACCATTATTATCATGACATTAACCTATAAAAATAGGCGTATCACGAGGCCCTTTCGTC
Amino acid sequence of human glucocerebrosidase without
signal peptide sequence (as encoded by SEQ ID NO: 1)
SEQ ID NO: 5
ARPCIPKSFGYSSVVCVCNATYCDSFDPPTFPALGTFSRYESTRSGRRMELSMGPIQANHTGTGLLLT
LQPEQKFQKVKGFGGAMTDAAALNILALSPPAQNLLLKSYFSEEGIGYNIIRVPMASCDFSIRTYTYA
DTPDDFQLHNFSLPEEDTKLKIPLIHRALQLAQRPVSLLASPWTSPTWLKTNGAVNGKGSLKGQPGDI
YHQTWARYFVKFLDAYAEHKLQFWAVTAENEPSAGLLSGYPFQCLGFTPEHQRDFIARDLGPTLANST
HHNVRLLMLDDQRLLLPHWAKVVLTDPEAAKYVHGIAVHWYLDFLAPAKATLGETHRLFPNTMLFASE
ACVGSKFWEQSVRLGSWDRGMQYSHSIITNLLYHVVGWTDWNLALNPEGGPNWVRNFVDSPIIVDITK
DTFYKQPMFYHLGHFSKFIPEGSQRVGLVASQKNDLDAVALMHPDGSAVVVVLNRSSKDVPLTIKDPA
VGFLETISPGYSIHTYLWRRQ
Full amino acid sequence of human glucocerebrosidase
including short signal peptide (as encoded by SEQ ID NO: 1)
SEQ ID NO: 6
MAGSLTGLLLLQAVSWASGARPCIPKSFGYSSVVCVCNATYCDSFDPPTFPALGTFSRYESTRSGRRM
ELSMGPIQANHTGTGLLLTLQPEQKFQKVKGFGGAMTDAAALNILALSPPAQNLLLKSYFSEEGIGYN
IIRVPMASCDFSIRTYTYADTPDDFQLHNFSLPEEDTKLKIPLIHRALQLAQRPVSLLASPWTSPTWL
KTNGAVNGKGSLKGQPGDIYHQTWARYFVKFLDAYAEHKLQFWAVTAENEPSAGLLSGYPFQCLGFTP
EHQRDFIARDLGPTLANSTHHNVRLLMLDDQRLLLPHWAKVVLTDPEAAKYVHGIAVHWYLDFLAPAK
ATLGETHRLFPNTMLFASEACVGSKFWEQSVRLGSWDRGMQYSHSIITNLLYHVVGWTDWNLALNPEG
GPNWVRNFVDSPIIVDITKDTFYKQPMFYHLGHFSKFIPEGSQRVGLVASQKNDLDAVALMHPDGSAV
VVVLNRSSKDVPLTIKDPAVGFLETISPGYSIHTYLWRRQ
Full nucleotide sequence of coding sequence of human GBA1 gene (wild type)
Human glucocerebrosidase mRNA, complete cds
GenBank: M19285.1
>M19285.1: 123-1733 Human glucocerebrosidase mRNA, complete cds
SEQ ID NO: 7
ATGGAGTTTTCAAGTCCTTCCAGAGAGGAATGTCCCAAGCCTTTGAGTAGGGTAAGCATCATGGCTGG
CAGCCTCACAGGTTTGCTICTACTTCAGGCAGTGTCGTGGGCATCAGGTGCCCGCCCCTGCATCCCTA
AAAGCTTCGGCTACAGCTCGGTGGTGTGTGTCTGCAATGCCACATACTGTGACTCCTTTGACCCCCCG
ACCTTTCCTGCCCTTGGTACCTTCAGCCGCTATGAGAGTACACGCAGTGGGCGACGGATGGAGCTGAG
TATGGGGCCCATCCAGGCTAATCACACGGGCACAGGCCTGCTACTGACCCTGCAGCCAGAACAGAAGT
TCCAGAAAGTGAAGGGATTTGGAGGGGCCATGACAGATGCTGCTGCTCTCAACATCCTTGCCCTGTCA
CCCCCTGCCCAAAATTTGCTACTTAAATCGTACTTCTCTGAAGAAGGAATCGGATATAACATCATCCG
GGTACCCATGGCCAGCTGTGACTTCTCCATCCGCACCTACACCTATGCAGACACCCCTGATGATTTCC
AGTTGCACAACTTCAGCCTCCCAGAGGAAGATACCAAGCTCAAGATACCCCTGATTCACCGAGCCCTG
CAGTTGGCCCAGCGTCCCGTTTCACTCCTTGCCAGCCCCTGGACATCACCCACTTGGCTCAAGACCAA
TGGAGCGGTGAATGGGAAGGGGTCACTCAAGGGACAGCCCGGAGACATCTACCACCAGACCTGGGCCA
GATACTTTGTGAAGTTCCTGGATGCCTATGCTGAGCACAAGTTACAGTTCTGGGCAGTGACAGCTGAA
AATGAGCCTTCTGCTGGGCTGTTGAGTGGATACCCCTTCCAGTGCCTGGGCTTCACCCCTGAACATCA
GCGAGACTTCATTGCCCGTGACCTAGGTCCTACCCTCGCCAACAGTACTCACCACAATGTCCGCCTAC
TCATGCTGGATGACCAACGCTTGCTGCTGCCCCACTGGGCAAAGGTGGTACTGACAGACCCAGAAGCA
GCTAAATATGTTCATGGCATTGCTGTACATTGGTACCTGGACTTTCTGGCTCCAGCCAAAGCCACCCT
AGGGGAGACACACCGCCTGTTCCCCAACACCATGCTCTTTGCCTCAGAGGCCTGTGTGGGCTCCAAGT
TCTGGGAGCAGAGIGTGCGGCTAGGCTCCTGGGATCGAGGGATGCAGTACAGCCACAGCATCATCACG
AACCTCCTGTACCATGTGGTCGGCTGGACCGACTGGAACCTTGCCCTGAACCCCGAAGGAGGACCCAA
TTGGGTGCGTAACTTTGTCGACAGTCCCATCATTGTAGACATCACCAAGGACACGTTTTACAAACAGC
CCATGTTCTACCACCTTGGCCACTTCAGCAAGTTCATTCCTGAGGGCTCCCAGAGAGTGGGGCTGGTT
GCCAGTCAGAAGAACGACCTGGACGCAGTGGCACTGATGCATCCCGATGGCTCTGCTGTTGTGGTCGT
GCTAAACCGCTCCTCTAAGGATGTGCCTCTTACCATCAAGGATCCTGCTGTGGGCTTCCTGGAGACAA
TCTCACCTGGCTACTCCATTCACACCTACCTGTGGCATCGCCAGTGA
Full amino acid sequence of human glucocerebrosidase including
long signal peptide
SEQ ID NO: 8
MEFSSPSREECPKPLSRVSIMAGSLTGLLLLQAVSWASGARPCIPKSFGYSSVVCVCNATYCDSFDPP
TFPALGTFSRYESTRSGRRMELSMGPIQANHTGTGLLLTLQPEQKFQKVKGFGGAMTDAAALNILALS
PPAQNLLLKSYFSEEGIGYNIIRVPMASCDFSIRTYTYADTPDDFQLHNFSLPEEDTKLKIPLIHRAL
QLAQRPVSLLASPWTSPTWLKTNGAVNGKGSLKGQPGDIYHQTWARYFVKFLDAYAEHKLQFWAVTAE
NEPSAGLLSGYPFQCLGFTPEHQRDFIARDLGPTLANSTHHNVRLLMLDDQRLLLPHWAKVVLTDPEA
AKYVHGIAVHWYLDFLAPAKATLGETHRLFPNTMLFASEACVGSKFWEQSVRLGSWDRGMQYSHSIIT
NLLYHVVGWTDWNLALNPEGGPNWVRNFVDSPIIVDITKDTFYKQPMFYHLGHFSKFIPEGSQRVGLV
ASQKNDLDAVALMHPDGSAVVVVLNRSSKDVPLTIKDPAVGFLETISPGYSIHTYLWRRQ
Nucleotide sequence of the CAG promoter
SEQ ID NO: 9
ctagttattaatagtaatcaattacggggtcattagttcatagcccatatatggagttccgcgttaca
taacttacggtaaatggcccgcctggctgaccgcccaacgacccccgcccattgacgtcaataatgac
gtatgttcccatagtaacgccaatagggactttccattgacgtcaatgggtggagtatttacggtaaa
ctgcccacttggcagtacatcaagtgtatcatatgccaagtacgccccctattgacgtcaatgacggt
aaatggcccgcctggcattatgcccagtacatgaccttatgggactttcctacttggcagtacatcta
cgtattagtcatcgctattaccatggtcgaggtgagccccacgttctgcttcactctccccatctccc
ccccctccccacccccaattttgtatttatttattttttaattattttgtgcagcgatgggggcgggg
ggggggggggggcgcgcgccaggcggggcggggcggggcgaggggcggggcggggcgaggcggagagg
tgcggcggcagccaatcagagcggcgcgctccgaaagtttccttttatggcgaggcggcggcggcggc
ggccctataaaaagcgaagcgcgcggcgggcg
GFP encoding nucleotide sequence
SEQ ID NO: 10
Atggtgagcaagggcgaggagctgttcaccggggtggtgcccatcctggtcgagctggacggcgacgt
aaacggccacaagttcagcgtgtccggcgagggcgagggcgatgccacctacggcaagctgaccctga
agttcatctgcaccaccggcaagctgcccgtgccctggcccaccctcgtgaccaccctgacctacggc
gtgcagtgcttcagccgctaccccgaccacatgaagcagcacgacttcttcaagtccgccatgcccga
aggctacgtccaggagcgcaccatcttcttcaaggacgacggcaactacaagacccgcgccgaggtga
agttcgagggcgacaccctggtgaaccgcatcgagctgaagggcatcgacttcaaggaggacggcaac
atcctggggcacaagctggagtacaactacaacagccacaacgtctatatcatggccgacaagcagaa
gaacggcatcaaggtgaacttcaagatccgccacaacatcgaggacggcagcgtgcagctcgccgacc
actaccagcagaacacccccatcggcgacggccccgtgctgctgcccgacaaccactacctgagcacc
cagtccgccctgagcaaagaccccaacgagaagcgcgatcacatggtcctgctggagttcgtgaccgc
cgccgggatcactctcggcatggacgagctgtacaagtga
Human GBA1 coding nucleotide sequence (IDT optimized sequence)
SEQ ID NO: 11
atggagttctcatctccctcacgagaagaatgtccgaaacctctttcaagagtaagcatcatggccgg
atggagttctcatctccctcacgagaagaatgtccgaaacctctttcaagagtaagcatcatggccgg
cagcttgaccggtcttttgttgttgcaggccgtgtcctgggcctcaggtgctaggccatgcattccta
aatccttcggctatagtagcgtggtttgcgtctgcaacgccacatactgtgacagtttcgatccacct
accttcccagcgctgggtaccttctcacggtatgaatcaacgcgatcagggcgcagaatggaactttc
aatggggccaatccaagctaaccacacgggaacgggtcttctgctgacgctccaaccggaacaaaagt
tccaaaaggtaaaaggctttggaggtgcgatgactgatgccgcagcactcaacatcctggcgctctca
ccgccggcacaaaatttgctgttgaagagttatttctcagaagaagggatcggttacaacatcatacg
ggtcccgatggcgagctgtgacttttctataagaacatatacctatgcggatacgcccgacgatttcc
aacttcataattttagtctgcctgaggaagacacaaagttgaagataccgctgatacacagagcattg
cagcttgctcaacgaccggtcagcttgcttgccagcccatggacaagtccaacatggcttaagaccaa
tggcgcggttaatggcaagggatccctgaagggccagccgggagacatctatcatcaaacttgggcgc
ggtattttgtcaagttcttggacgcctacgctgagcacaaactgcagttctgggccgttaccgccgaa
aatgaaccatccgccggactgctttctggctaccctttccaatgtcttggctttacgcctgaacacca
aagagacttcattgctcgggaccttggtccaacgctcgcgaacagtactcatcataatgtacgactct
tgatgctcgatgaccagcgactgttgcttccacattgggccaaggtagttctgaccgaccccgaagcc
gctaaatacgtccacggcattgctgtccattggtaccttgactttttggctcccgcaaaagccactct
gggtgaaacacacagactctttccaaacacgatgcttttcgcatcagaagcctgcgtcggaagtaaat
tttgggaacagtcagtaaggttgggtagttgggatcgcgggatgcaatatagtcatagcattattacc
aacttgctttatcacgtcgttgggtggacagattggaacctcgcgttgaatcctgaaggcggccctaa
ttgggtaagaaactttgttgattcacctattatcgtcgacataaccaaggacacattctacaagcaac
cgatgttctatcaccttgggcatttcagtaaattcataccagagggcagccagcgcgtcgggttggta
gcctctcaaaaaaacgatttggatgcggtcgctctgatgcatcccgacgggagcgcagtagtcgttgt
ccttaaccgaagctccaaggatgtacccctcacgattaaggaccctgctgtcgggttccttgaaacta
taagtcccggctatagtattcatacttatctctggagaagacagtga
Human GBA1 coding nucleotide sequence (GenScript optimized sequence)
SEQ ID NO: 12
ATGGAGTTTTCAAGCCCCTCACGGGAAGAGTGCCCTAAGCCCCTGTCACGGGTCTCAATTATGGCCGG
GAGCCTGACTGGCCTGCTGCTGCTGCAGGCCGTGAGCTGGGCATCAGGAGCCAGGCCTTGCATCCCAA
AGTCTTTCGGCTACAGCTCCGTGGTGTGCGTGTGCAACGCCACCTATTGTGACTCCTTCGATCCCCCT
ACCTTTCCCGCCCTGGGCACATTTTCTAGATACGAGTCTACACGCAGCGGCCGGAGAATGGAGCTGAG
CATGGGCCCTATCCAGGCCAATCACACCGGAACAGGCCTGCTGCTGACCCTGCAGCCAGAGCAGAAGT
TCCAGAAGGTGAAGGGCTTTGGAGGAGCAATGACAGACGCAGCCGCCCTGAACATCCTGGCCCTGTCC
CCACCCGCCCAGAATCTGCTGCTGAAGTCCTACTTCTCTGAGGAGGGCATCGGCTATAACATCATCCG
GGTGCCCATGGCCAGCTGCGACTTTTCCATCAGAACCTACACATATGCCGATACCCCTGACGATTTCC
AGCTGCACAATTTTTCCCTGCCAGAGGAGGATACAAAGCTGAAGATCCCCCTGATCCACCGGGCCCTG
CAGCTGGCACAGCGGCCCGTGAGCCTGCTGGCCAGCCCCTGGACCTCCCCTACATGGCTGAAGACCAA
CGGCGCCGTGAATGGCAAGGGCTCTCTGAAGGGACAGCCAGGCGACATCTACCACCAGACATGGGCCA
GATATTTCGTGAAGTTTCTGGATGCCTACGCCGAGCACAAGCTGCAGTTCTGGGCCGTGACCGCAGAG
AACGAGCCTTCTGCCGGCCTGCTGAGCGGCTATCCCTTCCAGTGCCTGGGCTTTACACCTGAGCACCA
GCGGGACTTTATCGCCAGAGATCTGGGCCCAACCCTGGCCAACTCCACACACCACAATGTGAGGCTGC
TGATGCTGGACGATCAGCGCCTGCTGCTGCCTCACTGGGCCAAGGTGGTGCTGACCGACCCAGAGGCC
GCCAAGTACGTGCACGGCATCGCCGTGCACTGGTATCTGGATTTCCTGGCACCAGCAAAGGCCACCCT
GGGAGAGACACACCGGCTGTTCCCTAACACCATGCTGTTTGCCAGCGAGGCCTGCGTGGGCTCCAAGT
TTTGGGAGCAGTCCGTGAGGCTGGGATCTTGGGACAGGGGCATGCAGTACTCCCACTCTATCATCACC
AATCTGCTGTATCACGTGGTGGGCTGGACAGACTGGAACCTGGCCCTGAATCCAGAGGGCGGCCCCAA
CTGGGTGAGAAATTTCGTGGATAGCCCCATCATCGTGGACATCACCAAGGATACATTCTACAAGCAGC
CAATGTTTTATCACCTGGGCCACTTCTCTAAGTTTATCCCAGAGGGCAGCCAGAGGGTGGGCCTGGTG
GCCAGCCAGAAGAACGACCTGGATGCCGTGGCCCTGATGCACCCTGATGGCTCCGCCGTGGTGGTGGT
GCTGAATCGCTCTAGCAAGGACGTGCCTCTGACCATCAAGGATCCAGCCGTGGGCTTCCTGGAGACTA
TTTCCCCCGGCTATTCAATTCATACCTATCTGTGGAGAAGGCAGTGA
Nucleotide sequence of the hSyn promoter (NCBI ref. NG_008437.1)
SEQ ID NO: 13
AGTGCAAGTGGGTTTTAGGACCAGGATGAGGCGGGGTGGGGGTGCCTACCTGACGACCGACCCCGACC
CACTGGACAAGCACCCAACCCCCATTCCCCAAATTGCGCATCCCCTATCAGAGAGGGGGAGGGGAAAC
AGGATGCGGCGAGGCGCGTGCGCACTGCCAGCTTCAGCACCGCGGACAGTGCCTTCGCCCCCGCCTGG
CGGCGCGCGCCACCGCCGCCTCAGCACTGAAGGCGCGCTGACGTCACTCGCCGGTCCCCCGCAAACTC
CCCTTCCCGGCCACCTTGGTCGCGTCCGCGCCGCCGCCGGCCCAGCCGGACCGCACCACGCGAGGCGC
GAGATAGGGGGGCACGGGCGCGACCATCTGCGCTGCGGCGCCGGCGACTCAGCGCTGCCTCAGTCTGC
GGTGGGCAGCGGAGGAGTCGTGTCGTGCCTGAGAGCGCAG
Amino acid sequence of AAV TT capsid protein
SEQ ID NO: 14
MAADGYLPDWLEDTLSEGIRQWWKLKPGPPPPKPAERHKDDSRGLVLPGYKYLGPFNGLDKGEPVNEA
DAAALEHDKAYDRQLDSGDNPYLKYNHADAEFQERLKEDTSFGGNLGRAVFQAKKRILEPLGLVEEPV
KTAPGKKRPVEHSPAEPDSSSGTGKSGQQPARKRLNFGQTGDADSVPDPQPLGQPPAAPSGLGTNTMA
SGSGAPMADNNEGADGVGNSSGNWHCDSTWMGDRVITTSTRTWALPTYNNHLYKQISSQSGASNDNHY
FGYSTPWGYFDFNRFHCHFSPRDWQRLINNNWGFRPKRLSFKLFNIQVKEVTQNDGTTTIANNLTSTV
QVFTDSEYQLPYVLGSAHQGCLPPFPADVFMVPQYGYLTLNNGSQAVGRSSFYCLEYFPSQMLRTGNN
FTFSYTFEDVPFHSSYAHSQSLDRLMNPLIDQYLYYLSRINTPSGTTTMSRLQFSQAGASDIRDQSRN
WLPGPCYRQQRVSKTAADNNNSDYSWTGATKYHLNGRDSLVNPGPAMASHKDDEEKYFPQSGVLIFGK
QDSGKTNVDIEKVMITDEEEIRTTNPVATEQYGSVSTNLQSGNTQAATSDVNTQGVLPGMVWQDRDVY
LQGPIWAKIPHTDGHFHPSPLMGGFGLKHPPPQILIKNTPVPANPSTTFSAAKFASFITQYSTGQVSV
EIEWELQKENSKRWNPEIQYTSNYNKSVNVDFTVDINGVYSEPRPIGTRYLTRNL
Nucleotide sequence of Flip ITR of AAV2
SEQ ID NO: 15
TTGGCCACTCCCTCTCTGCGCGCTCGCTCGCTCACTGAGGCCGGGCGACCAAAGGTCGCCCGACGCCC
GGGCTTTGCCCGGGCGGCCTCAGTGAGCGAGCGAGCGCGCAGAGAGGGAGTGGCCAACTCCATCACTA
GGGGTTCCT
Nucleotide sequence of Flop ITR of AAV2
SEQ ID NO: 16
AGGAACCCCTAGTGATGGAGTTGGCCACTCCCTCTCTGCGCGCTCGCTCGCTCACTGAGGCCGGGCGA
CCAAAGGTCGCCCGACGCCCGGGCTTTGCCCGGGCGGCCTCAGTGAGCGAGCGAGCGCGCAGAGAGGG
AGTGGCCAA
SEQ ID NO: 17: Human GBA1 isoform 2 amino acid sequence (NCBI Ref.
NP_001165282.1)
MELSMGPIQANHTGTGLLLTLQPEQKFQKVKGFGGAMTDAAALNILALSPPAQNLLLKSYFSEEGIGY
NIIRVPMASCDFSIRTYTYADTPDDFQLHNFSLPEEDTKLKIPLIHRALQLAQRPVSLLASPWTSPTW
LKTNGAVNGKGSLKGQPGDIYHQTWARYFVKFLDAYAEHKLQFWAVTAENEPSAGLLSGYPFQCLGFT
PEHQRDFIARDLGPTLANSTHHNVRLLMLDDQRLLLPHWAKVVLTDPEAAKYVHGIAVHWYLDFLAPA
KATLGETHRLFPNTMLFASEACVGSKFWEQSVRLGSWDRGMQYSHSIITNLLYHVVGWTDWNLALNPE
GGPNWVRNFVDSPIIVDITKDTFYKQPMFYHLGHFSKFIPEGSQRVGLVASQKNDLDAVALMHPDGSA
VVVVLNRSSKDVPLTIKDPAVGFLETISPGYSIHTYLWRRQ
SEQ ID NO: 18: Human GBA1 isoform 3 amino acid sequence (NCBI ref.
NP_001165283.1)
MEFSSPSREECPKPLSRVSIMAGSLTGLLLLQAVSWASGARPCIPKSFGYSSVVCVCNATYCDSFDPP
TFPALGTFSRYESTRSGRRMELSMGPIQANHTGTGIGYNIIRVPMASCDFSIRTYTYADTPDDFQLHN
FSLPEEDTKLKIPLIHRALQLAQRPVSLLASPWTSPTWLKTNGAVNGKGSLKGQPGDIYHQTWARYFV
KFLDAYAEHKLQFWAVTAENEPSAGLLSGYPFQCLGFTPEHQRDFIARDLGPTLANSTHHNVRLLMLD
DQRLLLPHWAKVVLTDPEAAKYVHGIAVHWYLDFLAPAKATLGETHRLFPNTMLFASEACVGSKFWEQ
SVRLGSWDRGMQYSHSIITNLLYHVVGWTDWNLALNPEGGPNWVRNFVDSPIIVDITKDTFYKQPMFY
HLGHFSKFIPEGSQRVGLVASQKNDLDAVALMHPDGSAVVVVLNRSSKDVPLTIKDPAVGFLETISPG
YSIHTYLWRRQ
SEQ ID NO: 19: Full nucleotide sequence of coding sequence of human
GBA1 gene
atggagttttcaagtccttccagagaggaatgtcccaagcctttgagtagggtaagcatcatggctgg
cagcctcacaggattgcttctacttcaggcagtgtcgtgggcatcaggtgcccgcccctgcatcccta
aaagcttcggctacagctcggtggtgtgtgtctgcaatgccacatactgtgactcctttgaccccccg
acctttcctgcccttggtaccttcagccgctatgagagtacacgcagtgggcgacggatggagctgag
tatggggcccatccaggctaatcacacgggcacaggcctgctactgaccctgcagccagaacagaagt
tccagaaagtgaagggatttggaggggccatgacagatgctgctgctctcaacatccttgccctgtca
ccccctgcccaaaatttgctacttaaatcgtacttctctgaagaaggaatcggatataacatcatccg
ggtacccatggccagctgtgacttctccatccgcacctacacctatgcagacacccctgatgatttcc
agttgcacaacttcagcctcccagaggaagataccaagctcaagatacccctgattcaccgagccctg
cagttggcccagcgtcccgtttcactccttgccagcccctggacatcacccacttggctcaagaccaa
tggagcggtgaatgggaaggggtcactcaagggacagcccggagacatctaccaccagacctgggcca
gatactttgtgaagttcctggatgcctatgctgagcacaagttacagttctgggcagtgacagctgaa
aatgagccttctgctgggctgttgagtggataccccttccagtgcctgggcttcacccctgaacatca
gcgagacttcattgcccgtgacctaggtcctaccctcgccaacagtactcaccacaatgtccgcctac
tcatgctggatgaccaacgcttgctgctgccccactgggcaaaggtggtactgacagacccagaagca
gctaaatatgttcatggcattgctgtacattggtacctggactttctggctccagccaaagccaccct
aggggagacacaccgcctgttccccaacaccatgctctttgcctcagaggcctgtgtgggctccaagt
tctgggagcagagtgtgcggctaggctcctgggatcgagggatgcagtacagccacagcatcatcacg
aacctcctgtaccatgtggtcggctggaccgactggaaccttgccctgaaccccgaaggaggacccaa
ttgggtgcgtaactttgtcgacagtcccatcattgtagacatcaccaaggacacgttttacaaacagc
ccatgttctaccaccttggccacttcagcaagttcattcctgagggctcccagagagtggggctggtt
gccagtcagaagaacgacctggacgcagtggcactgatgcatcccgatggctctgctgttgtggtcgt
gctaaaccgctcctctaaggatgtgcctcttaccatcaaggatcctgctgtgggcttctggagaca
tctcacctggctactccattcacacctacctgtggcgtcgccagtga
SEQ ID NO: 20: nGUSB promoter nucleotide sequence #2
ATTCCTGCTGGGAAAAGCAAGTGGAGGTGCTCCTTGAAGAAACAGGGGGATCCCACCGATCTCAGGGG
TTCTGTTCTGGCCTGCGGCCCTGGATCGTCCAGCCTGGGTCGGGGTGGGGAGCAGACCTCGCCCTTAT
CGGCTGGGGCTGAGGGTGAGGGTCCCGTTTCCCCAAAGGCCTAGCCTGGGGTTCCAGCCACAAGCCCT
ACCGGGCAGCGCCCGGCCCCGCCCCTCCAGGCCTGGCACTCGTCCTCAACCAAGATGGCGCGGATGGC
TTCAGGCGCATCACGACACCGGCGCGTCACGCGACCCGCCCTACGGGCACCTCCCGCGCTTTTCTTAG
CGCCGCAGACGGTGGCCGAGCGGGGGACCGGGAAGC
Nucleotide sequence of the CAG promoter # 2
SEQ ID NO: 21
ctagttattaatagtaatcaattacggggtcattagttcatagcccatatatggagttccgcgttaca
taacttacggtaaatggcccgcctggctgaccgcccaacgacccccgcccattgacgtcaataatgac
gtatgttcccatagtaacgccaatagggactttccattgacgtcaatgggtggagtatttacggtaaa
ctgcccacttggcagtacatcaagtgtatcatatgccaagtacgccccctattgacgtcaatgacggt
aaatggcccgcctggcattatgcccagtacatgaccttatgggactttcctacttggcagtacatcta
cgtattagtcatcgctattaccatggtcgaggtgagccccacgttctgcttcactctccccatctccc
ccccctccccacccccaattttgtatttatttattttttaattattttgtgcagcgatggggcggggg
ggggggggggggcgcgcgccaggcggggcggggcggggcgaggggcggggcggggcgaggcggagagg
tgcggcggcagccaatcagagcggcgcgctccgaaagtttccttttatggcgaggcggcggcggcggc
ggccctataaaaagcgaagcgcgcggcgggcgggagtcgctgcgacgctgccttcgccccgtgccccg
ctccgccgccgcctcgcgccgcccgccccggctctgactgaccgcgttactcccacaggtgagcgggc
gggacggcccttctcctccgggctgtaattagcgcttggtttaatgacggcttgtttcttttctgtgg
ctgcgtgaaagccttgaggggctccgggagggccctttgtgcgggggggagcggctcggggggtgcgt
gcgtgtgtgtgtgcgtggggagcgccgcgtgcggcccgcgctgcccggcggctgtgagcgctgcgggc
gcggcgcggggctttgtgcgctccgcagtgtgcgcgaggggagcgcggccgggggcggtgccccgcgg
tgcggggggggctgcgaggggaacaaaggctgcgtgcggggtgtgtgcgtgggggggtgagcaggggg
tgtgggcgcggcggtcgggctgtaacccccccctgcacccccctccccgagttgctgagcacggcccg
gcttcgggtgcggggctccgtacggggcgtggcgcggggctcgccgtgccgggcggggggtggcggca
ggtgggggtgccgggcggggcggggccgcctcgggccggggagggctcgggggaggggcgcggcggcc
cccggagcgccggcggctgtcgaggcgcggcgagccgcagccattgccttttatggtaatcgtgcgag
agggcgcagggacttcctttgtcccaaatctgtgcggagccgaaatctgggaggcgccgccgcacccc
ctctagcgggcgcggggcgaagcggtgcggcgccggcaggaaggaaatgggcggggagggccttcgtg
cgtcgccgcgccgccgtccccttctccctctccagcctcggggctgtccgcggggggacggctgcctt
cgggggggacggggcagggcggggttcggcttctggcgtgtgaccggcggctctagagcctctgctaa
ccatgttcatgccttcttctttttcctacag
Nucleotide sequence of the Human Ubiquitin C (UbC) promoter short version
SEQ ID NO: 22
Ggcctccgcgccgggttttggcgcctcccgcgggcgcccccctcctcacggcgagcgctgccacgtca
gacgaagggcgcagcgagcgtcctgatccttccgcccggacgctcaggacagcggcccgctgctcata
agactcggccttagaaccccagtatcagcagaaggacattttaggacgggacttgggtgactctaggg
cactggttttctttccagagagcggaacaggcgaggaaaagtagtcccttctcggcgattctgcggag
ggatctccgtggggcggtgaacgccgatgattatataaggacgcgccgggtgtggcacagctagttcc
gtcgcagccgggatttgggtcgcggttcttgtttgtggatcgctgtgatcgtcacttggt
Nucleotide sequence of the Human Ubiquitin C (UbC) promoter
long version
SEQ ID NO: 23
ccggaggcgcggcccaaaaccgcggagggcgcccgcggggggaggagtgccgctcgcgacggtgcagt
ctgcttcccgcgtcgctcgcaggactaggaaggcgggcctgcgagtcctgtcgccgggcgacgagtat
tctgagccggaatcttggggtcatagtcgtcttcctgtaaaatcctgccctgaacccactgagatccc
gtgaccaaaagaaaggtctctcgccttgtccgctccttttcatcagggaagagccgctaagacgcctc
cctagaggcaccccgccacttgcggctactaatatattcctgcgcggcccacaccgtgtcgatcaagg
cagcgtcggccctaaacccagcgccaagaacaaacacctagcgacactagcagtgaaccactcatcgc
ccgacgacccgaccggccccgaaagcaccggcggcccggcgagccaccctgccttcgcacacctctct
ggcggttcccgacatcagacccaggcgctcgttccaacgggacttgacccccaacccccctcgcgtcg
ttttaccgccgacaagggctcagaacttaccttctgcgaacactccgcccgacactccagcaactttg
ttccaccccccgtaccacccgccgttcttgggttccagaactccggaagcgattacgccctttcgaga
ataagcccactctacccgaccccgtggtagacccctgggactgcacttcaaacagtgactgacctctt
gagccaaacagcagacaacgcccccgccgtcaataccgccacggcaacccgtcacgtgggcatggaaa
ccctcgcgcgcgggagcagcacagcactgcagtgggcaagacaaccgaatattacgtcccaccccggt
ggacggccatccacacgccatccgaaaagaggcagcgtcctgcgtcccaagcccggatcccatccgag
aggacttagctgtccgcggcctggagaccactcccctccctattcactccgcagtcaaagaaaccagc
caaaatacatggatagaagaattcatcgacttcgaggccaaaacttgatacgcgagccccaaccgctc
acacaaaacacttcaaaaaatccgtggaaaactttacattagtaaacccagttatacattaaaagtca
caatctgatcatttaacaggcgatttaagaccggcaaaaaccgaaaaaacaatctg
Nucleotide sequence of the Phosphoglycerate kinase 1 (PGK) promoter
SEQ ID NO: 24
gacccctctctccagccactaagccagttgctccctcggctgacggctgcacgcgaggcctccgaacg
tcttacgccttgtggcgcgcccgtccttgtcccgggtgtgatggcggggtgtggggcggagggcgtgg
cggggaagggccggcgacgagagccgcgcgggacgactcgtcggcgataaccggtgtcgggtagcgcc
agccgcgcgacggtaacgagggaccgcgacaggcagacgctcccatgatcactctgcacgccgaaggc
aaacagtgcaggccgtgcggcgcttggcgttccttggaagggctgaatccccgcctcgtccttcgcag
cggccccccgggtgttcccatcgccgcttctaggcccactgcgacgcttgcctgcacttcttacacgc
tctgggtcccagccgcggcgacgcaaagggccttggtgcgggtctcgtcggcgcagggacgcgtttgg
gtcccgacggaaccttttccgcgttggggttgggg
Nucleotide sequence of the CBA/CBh promoter #1
SEQ ID NO: 25
AGATGTACTGCCAAGTAGGAAAGTCCCGTAAGGTCATGTACTGGGCATAATGCCAGGCGGGCCATTTA
CCGTCATTGACGTCAATAGGGGGCGTACTTGGCATATGATACACTTGATGTACTGCCAAGTGGGCAGT
TTACCGTAAATACTCCACCCATTGACGTCAATGGAAAGTCCCTATTGGCGTTACTATGGGAACATACG
TCATTATTGACGTCAATGGGCGGGGGTCGTTGGGCGGTCAGCCAGGCGGGCCATTTACCGTAAGTTAT
GTAACGCGGAACTCCATATATGGGCTATGAACTAATGACCCCGTAATTGATTACTATTAACCACGTTC
TGCTTCACTCTCCCCATCTCCCCCCCCTCCCCACCCCCAATTTTGTATTTATTTATTTTTTAATTATT
TTGTGCAGCGATGGGGGCGGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTCCGAAAGTTTCCTTTTAT
GGCGAGGCGGCGGCGGCGGCGGCCCTATAAAAAGCGAAGCGCGCGGCGGAACTGAAAAACCAGAAAGT
TAACTGGTAAGTTTAGTCTTTTTGTCTTTTATTTCAGGTCCTGGTGGTGCAAATCAAAGAACTGCTCC
TCAGTGGATGTTGCCTTTACTTCTAGGCCTGTACGGAAGTGTTACTTCTGCTCTAAAAGCT
Nucleotide sequence of the CBA/CBh promoter #2
SEQ ID NO: 26
AGATGTACTGCCAAGTAGGAAAGTCCCGTAAGGTCATGTACTGGGCATAATGCCAGGCGGGCCATTTA
CCGTCATTGACGTCAATAGGGGGCGTACTTGGCATATGATACACTTGATGTACTGCCAAGTGGGCAGT
TTACCGTAAATACTCCACCCATTGACGTCAATGGAAAGTCCCTATTGGCGTTACTATGGGAACATACG
TCATTATTGACGTCAATGGGCGGGGGTCGTTGGGCGGTCAGCCAGGCGGGCCATTTACCGTAAGTTAT
GTAACGCGGAACTCCATATATGGGCTATGAACTAATGACCCCGTAATTGATTACTATTAACCACGTTC
TGCTTCACTCTCCCCATCTCCCCCCCCTCCCCACCCCCAATTTTGTATTTATTTATTTTTTAATTATT
TTGTGCAGCGATGGGGGCGGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTCCGAAAGTTTCCTTTTAT
GGCGAGGCGGCGGCGGCGGCGGCCCTATAAAAAGCGAAGCGCGCGGCGGGGAGTCGCTGCGTTGCCTT
CGCCCCGTGCCCCGCTCCGCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCAAGAGGTAAGGGTTTAAGGGAT
GGTTGGTTGGTGGGGTATTAATGTTTAATTACCTGTTTTACAGGCCTGAAATCACTTGGTTTTAGGTT
GG
GFP encoding nucleotide sequence #2
SEQ ID NO: 27
atggtgagcaagggcgaggagctgttcaccggggtggtgcccatcctggtcgagctggacggcgacgt
aaacggccacaagttcagcgtgtccggcgagggcgagggcgatgccacctacggcaagctgaccctga
agttcatctgcaccaccggcaagctgcccgtgccctggcccaccctcgtgaccaccctgacctacggc
gtgcagtgcttcagccgctaccccgaccacatgaagcagcacgacttcttcaagtccgccatgcccga
aggctacgtccaggagcgcaccatcttcttcaaggacgacggcaactacaagacccgcgccgaggtga
agttcgaggacgacaccctggtgaaccgcatcgagctgaagggcatcgacttcaaggaggacggcaac
atcctggggcacaagctggagtacaactacaacagccacaacgtctatatcatggccgacaagcagaa
gaacggcatcaaggtgaacttcaagatccgccacaacatcgaggacggcagcgtgcagctcgccgacc
actaccagcagaacacccccatcggcgacggccccgtgctgctgcccgacaaccactacctgagcacc
cagtccgccctgagcaaagaccccaacgagaagcgcgatcacatggtcctgctggagttcgtgaccgc
cgccgggatcactctcggcatggacgagctgtacaagtaa
Nucleotide sequence of the Human Ubiquitin C (UbC) promoter
long version #2
SEQ ID NO: 28
ggcctccgcgccgggttttggcgcctcccgcgggcgcccccctcctcacggcgagcgctgccacgtca
gacgaagggcgcagcgagcgtcctgatccttccgcccggacgctcaggacagcggcccgctgctcata
agactcggccttagaaccccagtatcagcagaaggacattttaggacgggacttgggtgactctaggg
cactggttttctttccagagagcggaacaggcgaggaaaagtagtcccttctcggcgattctgcggag
ggatctccgtggggcggtgaacgccgatgattatataaggacgcgccgggtgtggcacagctagttcc
gtcgcagccgggatttgggtcgcggttcttgtttgtggatcgctgtgatcgtcacttggtgagtagcg
ggctgctgggctggccggggctttcgtggccgccgggccgctcggtgggacggaagcgtgtggagaga
ccgccaagggctgtagtctgggtccgcgagcaaggttgccctgaactgggggttggggggagcgcagc
aaaatggcggctgttcccgagtcttgaatggaagacgcttgtgaggcgggctgtgaggtcgttgaaac
aaggtggggggcatggtgggcggcaagaacccaaggtcttgaggccttcgctaatgcgggaaagctct
tattcgggtgagatgggctggggcaccatctggggaccctgacgtgaagtttgtcactgactggagaa
ctcggtttgtcgtctgttgcgggggcggcagttatggcggtgccgttgggcagtgcacccgtaccttt
gggagcgcgcgccctcgtcgtgtcgtgacgtcacccgttctgttggcttataatgcagggtggggcca
cctgccggtaggtgtgcggtaggcttttctccgtcgcaggacgcagggttcgggcctagggtaggctc
tcctgaatcgacaggcgccggacctctggtgaggggagggataagtgaggcgtcagtttctttggtcg
gttttatgtacctatcttcttaagtagctgaagctccggttttgaactatgcgctcggggttggcgag
tgtgttttgtgaagttttttaggcaccttttgaaatgtaatcatttgggtcaatatgtaattttcagt
gttagactagtaaattgtccgctaaattctggccgtttttggcttttttgttagac
Nucleotide sequence of the Phosphoglycerate kinase 1 (PGK) promoter #2
SEQ ID NO: 29
ggggttggggttgcgccttttccaaggcagccctgggtttgcgcagggacgcggctgctctgggcgtg
gttccgggaaacgcagcggcgccgaccctgggtctcgcacattcttcacgtccgttcgcagcgtcacc
cggatcttcgccgctacccttgtgggccccccggcgacgcttcctgctccgcccctaagtcgggaagg
ttccttgcggttcgcggcgtgccggacgtgacaaacggaagccgcacgtctcactagtaccctcgcag
acggacagcgccagggagcaatggcagcgcgccgaccgcgatgggctgtggccaatagcggctgctca
gcagggcgcgccgagagcagcggccgggaaggggcggtgcgggaggcggggtgtggggcggtagtgtg
ggccctgttcctgcccgcgcggtgttccgcattctgcaagcctccggagcgcacgtcggcagtcggct
ccctcgttgaccgaatcaccgacctctctccccag
Nucleotide sequence of the CBA/CBh promoter #3
SEQ ID NO: 30
cgttacataacttacggtaaatggcccgcctggctgaccgcccaacgacccccgcccattgacgtcaa
tagtaacgccaatagggactttccattgacgtcaatgggtggagtatttacggtaaactgcccacttg
gcagtacatcaagtgtatcatatgccaagtacgccccctattgacgtcaatgacggtaaatggcccgc
ctggcattgtgcccagtacatgaccttatgggactttcctacttggcagtacatctacgtattagtca
tcgctattaccatggtcgaggtgagccccacgttctgcttcactctccccatctcccccccctcccca
cccccaattttgtatttatttattttttaattattttgtgcagcgatgggggcggggggggggggggg
gcgcgcgccaggcggggcggggcggggcgaggggcggggcggggcgaggcggagaggtgcggcggcag
ccaatcagagcggcgcgctccgaaagtttccttttatggcgaggcggcggcggcggcggccctataaa
aagcgaagcgcgcggcgggcgggagtcgctgcgacgctgccttcgccccgtgccccgctccgccgccg
cctcgcgccgcccgccccggctctgactgaccgcgttactcccacaggtgagcgggcgggacggccct
tctcctccgggctgtaattagctgagcaagaggtaagggtttaagggatggttggttggtggggtatt
aatgtttaattacctggagcacctgcctgaaatcactttttttcag
Amino acid sequence of AAV2 capsid protein
SEQ ID NO: 31
MAADGYLPDWLEDTLSEGIRQWWKLKPGPPPPKPAERHKDDSRGLVLPGYKYLGPFNGLDKGEPVNEA
DAAALEHDKAYDRQLDSGDNPYLKYNHADAEFQERLKEDTSFGGNLGRAVFQAKKRVLEPLGLVEEPV
KTAPGKKRPVEHSPVEPDSSSGTGKAGQQPARKRLNFGQTGDADSVPDPQPLGQPPAAPSGLGTNTMA
TGSGAPMADNNEGADGVGNSSGNWHCDSTWMGDRVITTSTRTWALPTYNNHLYKQISSQSGASNDNHY
FGYSTPWGYFDFNRFHCHFSPRDWQRLINNNWGFRPKRLNFKLFNIQVKEVTQNDGTTTIANNLTSTV
QVFTDSEYQLPYVLGSAHQGCLPPFPADVFMVPQYGYLTLNNGSQAVGRSSFYCLEYFPSQMLRTGNN
FTFSYTFEDVPFHSSYAHSQSLDRLMNPLIDQYLYYLSRTNTPSGTTTQSRLQFSQAGASDIRDQSRN
WLPGPCYRQQRVSKTSADNNNSEYSWTGATKYHLNGRDSLVNPGPAMASHKDDEEKFFPQSGVLIFGK
QGSEKTNVDIEKVMITDEEEIRTTNPVATEQYGSVSTNLQRGNRQAATADVNTQGVLPGMVWQDRDVY
LQGPIWAKIPHTDGHFHPSPLMGGFGLKHPPPQILIKNTPVPANPSTTFSAAKFASFITQYSTGQVSV
EIEWELQKENSKRWNPEIQYTSNYNKSVNVDFTVDINGVYSEPRPIGTRYLTRNL
Amino acid sequence of AAV9 capsid protein
SEQ ID NO: 32
MAADGYLPDWLEDNLSEGIREWWALKPGAPQPKANQQHQDNARGLVLPGYKYLGPGNGLDKGEPVNAA
DAAALEHDKAYDQQLKAGDNPYLKYNHADAEFQERLKEDTSFGGNLGRAVFQAKKRLLEPLGLVEEAA
KTAPGKKRPVEQSPQEPDSSAGIGKSGAQPAKKRLNFGQTGDTESVPDPQPIGEPPAAPSGVGSLTMA
SGGGAPVADNNEGADGVGSSSGNWHCDSQWLGDRVITTSTRTWALPTYNNHLYKQISNSTSGGSSNDN
AYFGYSTPWGYFDFNRFHCHFSPRDWQRLINNNWGFRPKRLNFKLFNIQVKEVTDNNGVKTIANNLTS
TVQVFTDSDYQLPYVLGSAHEGCLPPFPADVFMIPQYGYLTLNDGSQAVGRSSFYCLEYFPSQMLRTG
NNFQFSYEFENVPFHSSYAHSQSLDRLMNPLIDQYLYYLSKTINGSGQNQQTLKFSVAGPSNMAVQGR
NYIPGPSYRQQRVSTTVTQNNNSEFAWPGASSWALNGRNSLMNPGPAMASHKEGEDRFFPLSGSLIFG
KQGTGRDNVDADKVMITNEEEIKTTNPVATESYGQVATNHQSAQAQAQTGWVQNQGILPGMVWQDRDV
YLQGPIWAKIPHTDGNFHPSPLMGGFGMKHPPPQILIKNTPVPADPPTAFNKDKLNSFITQYSTGQVS
VEIEWELQKENSKRWNPEIQYTSNYYKSNNVEFAVNTEGVYSEPRPIGTRYLTRNL

1. A viral particle comprising a nucleic acid construct comprising a transgene encoding a glucocerebrosidase.

2. The viral particle of claim 1, wherein said transgene comprises a coding sequence of human glucocerebrosidase selected from the group consisting of SEQ ID NO: 5, 6, 8, 17 and 18, typically a sequence selected from the group consisting of SEQ ID NO: 1, 7, 11, 12 and 19.

3. The viral particle of claim 1 or 2, wherein said nucleic acid construct further comprises a promoter operably-linked to said transgene and wherein said promoter preferably allows the expression of said transgene at least in neuronal and microglial cells of the substantia nigra pars compacta (SNc).

4. The viral particle of claim 3, wherein said promoter is an ubiquitous promoter, notably a promoter selected from the group consisting of GusB promoter of SEQ ID NO: 2 or 20, CAG promoter of SEQ ID NO: 9 or 21 and hSyn promoter of SEQ ID NO: 13.

5. The viral particle of any one of claims 1-4, wherein said viral particle simultaneously targets at least neurons and glial cells.

6. The viral particle of any one of claims 1-5, wherein said viral particle simultaneously targets at least neurons and glial cells located in the cerebral cortex and subcellular structures such as the nucleus basalis of Meynert, the substantia nigra pars compacta, the locus ceruleus, the hippocampal formation and the entorhinal cortex.

7. The viral particle of any one of claims 1-6, wherein said viral particle simultaneously targets at least neurons and microglial cells.

8. The viral particle of any one of claims 1-7, wherein said viral particle simultaneously targets at least dopaminergic neurons and microglial cells in the substantia nigra pars compacta.

9. The viral particle of any one of claims 1-8, wherein said viral particle is a recombinant Adeno-Associated Virus (rAAV) particle, preferably including capsid proteins selected from the group consisting of AAV2, AAV5, AAV9, AAV-MNM004, AAV-MNM008 and AAV-TT.

10. The viral particle of any one of claims 1 to 9, wherein said viral particle comprises AAV TT capsid proteins, which preferably comprises amino acid sequence of SEQ ID NO: 14 or sequence having at least 98.5%, preferably 99% or 99.5% identity with SEQ ID NO: 14.

11. The viral particle of any one of claims 1 to 10, wherein said nucleic acid construct further comprises a polyadenylation signal sequence, preferably a polyadenylation signal sequence of sequence SEQ ID NO: 3.

12. The viral particle of any one of claims 1 to 11, wherein said nucleic acid construct is comprised in a viral vector which further comprises a 5′ITR and a 3′ITR sequences, preferably a 5′ITR and a 3′ITR sequences of an adeno-associated virus, more preferably a 5′ITR and 3′ITR sequences from the AAV2 serotype which comprise or consist of sequence SEQ ID NO: 15 and/or 16 or a sequence having at least 80% or at least 90% of identity with SEQ ID NO: 15 and/or 16.

13. The viral particle according to any one of claims 1 to 12, wherein said viral vector comprises SEQ ID NO: 4 or a nucleic acid sequence having at least 80% or at least 90% of identity with SEQ ID NO: 4.

14. The viral particle according to any one of claims 1 to 13, wherein said nucleic acid construct comprises a coding sequence of human glucocerebrosidase under the control of a promoter, allowing expression of said human glucocerebrosidase in at least both dopaminergic neurons and microglial cells, wherein said viral particle is selected among viral particles that targets at least dopaminergic neurons and microglial cells of the substantia nigra pars compacta, typically AAV particles including capsid proteins selected from the group consisting of AAV2, AAV5, AAV9.

15. The viral particle according to any one of claims 1 to 13, wherein said nucleic acid construct comprises a coding sequence of human glucocerebrosidase under the control of a promoter, allowing expression of said human glucocerebrosidase in at least both dopaminergic neurons and microglial cells, preferably in at least neurons of the substantia nigra pars compacta and the cerebral cortex wherein said viral particle is selected among viral particles with retrograde transport, typically viral particles including AAV retro capsid proteins selected from the group consisting of AAV-MNM004, AAV-MNM008 and AAV-TT.

16. The viral particle of any one of claims 1 to 15, wherein said viral particle comprises capsid proteins capable of retrograde transport (AAVretro).

17. The viral particle of claim 16, wherein said viral particle is capable of disseminating in the cerebral cortex, preferably at least to the substantia nigra pars compacta and the cerebral cortex after parenchymal injection in the caudate or putamen nuclei of a non-human primate as determined in an in vivo dissemination assay.

18. An in vivo dissemination assay comprising the steps:
a. injecting into the post-commissural putamen of a non-human primate a rAAV comprising a transgene encoding a green fluorescent protein (GFP) by intraparenchymal injection, and,
b. counting the number of neurons in the cerebral cortex, preferably in the brain areas innervating the caudate putamen nuclei expressing GFP about one month post injection.

19. The in vivo dissemination assay according to claim 18 further comprises a step c) of comparing the number of neurons expressing GFP in the cerebral cortex, preferably in the brain areas innervating the caudate putamen nuclei with a control experiment performed by injecting into the post-commissural putamen of a non-human primate an AAV-TT comprising a transgene encoding a green fluorescent protein (GFP) by intraparenchymal injection.

20. The viral particle of any one of claims 1-17 selected among AAVretro which are capable to disseminating in the cerebral cortex, preferably to at least to the substantia nigra pars compacta and the cerebral cortex to at least the same level as AAV-TT as determined in an in vivo dissemination assay according to any of claims 18 to 19.

21. The viral particle according to any one of claims 1 to 17 and 20, wherein said AAVretro is selected from the group consisting of AAV-MNM004, AAV-MNM008 and AAV-TT.

22. The viral particle according to any one of claims 1 to 17 or 20 to 21 wherein said viral particle comprises AAV TT capsid protein, which preferably comprises amino acid sequence of SEQ ID NO: 14 or sequence having at least 98.5%, preferably 99% or 99.5% of identity with SEQ ID NO: 14.

23. The viral particle according to any one of claims 1 to 17 or 20 to 22 for use in therapy.

24. A viral particle according to any one of claims 1 to 17 or 20 to 22 for use in treating tauopathy by gene therapy in a subject in need thereof.

25. The viral particle for use according to claim 24, wherein said tauopathy is a human sporadic tauopathy.

26. The viral particle for use according to any one of claims 24-25, wherein said subject to be treated is selected among patients with advanced stages of tauopathy.

27. The viral particle for use according to claims 24 to 26, wherein said tauopathy is an Alzheimer's disease, typically sporadic Alzheimer's disease.

28. The viral particle for use according to claims 24 to 26, wherein said tauopathy is a clinical entity other than Alzheimer's disease, this comprising but not limited to progressive supranuclear palsy, corticobasal degeneration, frontotemporal dementia, and Pick's disease.

29. The viral particle for use according to any one of claims 23 to 28, wherein said viral vector is administered to said subject by intrathecal or intraparenchymal administration, the latter preferably to brain areas such as the cerebral cortex and subcellular structures such as the nucleus basalis of Meynert, the substantia nigra pars compacta, the locus ceruleus, the hippocampal formation and the entorhinal cortex.

30. The viral particle for use according to claim 29, wherein said viral vector is administered to said subject by intraparenchymal administration, preferably to the brain areas of the substantia nigra pars compacta and/or caudate putamen nuclei

31. The viral particle for use according to claim 29, wherein said viral vector is administered to said subject by intraparenchymal administration, preferably to the dentate gyrus of the hippocampal formation.

32. A method for treating a tauopathy, preferably sporadic tauopathy, in a subject in need thereof, said method comprising administering to said subject a therapeutically effective amount of a viral particle according to any one of claims 1 to 17 or 20 to 22.

33. The method for treating a tauopathy according to claim 32 wherein said subject to be treated is selected among patients with advanced stages of tauopathy.

34. The method for treating a tauopathy according to claim 32 or 33 wherein said tauopathy is Alzheimer's disease.

35. The method for treating a tauopathy according to claim 32 or 33 wherein said tauopathy is a clinical entity other than Alzheimer's disease, this comprising but not limited to progressive supranuclear palsy, corticobasal degeneration, frontotemporal dementia, and Pick's disease.

36. The method for treating tauopathy according to claims 32-35 wherein said viral particle is administered to said subject by intrathecal or intraparenchymal administration, the latter preferably to brain areas such as the cerebral cortex and subcellular structures such as the nucleus basalis of Meynert, the substantia nigra pars compacta, the locus ceruleus, the hippocampal formation and the entorhinal cortex.

37. The method for treating tauopathy according to claims 32-35 wherein said viral particle is administered to said subject by intraparenchymal administration, preferably to the brain area of the substantia nigra pars compacta, the caudate putamen nuclei or the dentate gyrus of the hippocampal formation.
